(19) United States
(12) Patent Application Publication
(10) Pub. No.: US 2012/0094939 A1
(43) Pub. Date: Apr. 19, 2012

(54) ANTI-VIRAL COMPOUNDS, COMPOSITIONS AND METHODS

(76) Inventors: Stuart L. Schreiber, Boston, MA (US); Raymond T. Chung, Chestnut Hill, MA (US); Lee F. Peng, Somerville, MA (US); Sun Suk Kim, Geumcheon-gu (KR); Sirinya Matchacheep, Cambridge, MA (US); John A. Porco, JR., Brookline, MA (US); Aaron B. Beeler, Brookline, MA (US)

(21) Appl. No.: 12/594,701

(22) PCT Filed: Apr. 4, 2008
(86) PCT No.: PCT/US08/59378

§ 371 Date: Sep. 30, 2010

Related U.S. Application Data
(60) Provisional application No. 60/921,972, filed on Apr. 5, 2007.

(51) Int. Cl. A61K 31/706; A61P 31/12; A61K 31/336; C07D 493/10; A61K 31/343; C07D 303/32; A61K 31/5025

(52) U.S. Cl. 514/23; 549/332; 514/248; 514/462; 514/475

(57) ABSTRACT

The present invention is directed to compounds of formulae (I) and (II) and pharmaceutically acceptable forms thereof; pharmaceutical compositions thereof; and methods of treating a viral infection, such as a hepatitis C virus (HCV) infection, by administering to a subject diagnosed with or being susceptible to the viral infection a compound of formulae (I) and (II), a pharmaceutically acceptable form thereof, or a pharmaceutical composition thereof. The present invention is also directed to high-throughput methods of identifying compounds able to modulate hepatitis C virus (HCV) replication activity.

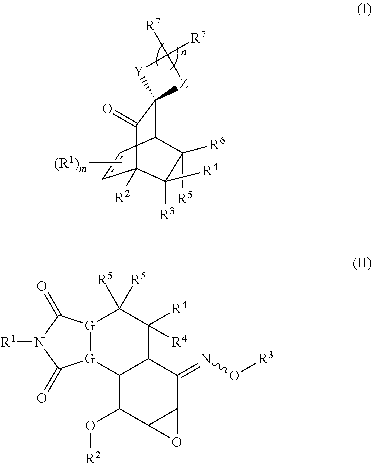

### PRIORITY INFORMATION

The present application claims priority under 35 U.S.C. §119(e) to U.S. provisional patent application, U.S. Ser. No. 60/921,972, filed Apr. 5, 2007, the entire contents of which are hereby incorporated by reference.

### GOVERNMENT FUNDING

This invention was made with Government support under N01-CO-12400 awarded by the National Cancer Institute's Initiative for Chemical Genetics, National Institutes of Health. The Government has certain rights in the invention.

### BACKGROUND OF THE INVENTION

Hepatitis C virus (HCV) infects over 170 million people worldwide and frequently leads to cirrhosis, liver failure, and hepatocellular carcinoma. Currently, the best therapy for the treatment of chronic hepatitis C is a combination of PEGylated interferon (PEG-IFN) and ribavirin. While the sustained virologic response (SVR) rate approaches 80% for patients with genotypes 2 and 3, the SVR rate is limited to about 45% for those with HCV genotype 1, which accounts for about 75% of all cases of HCV in the United States. Furthermore, interferon is parenteral, has an unfavorable side-effect profile, and its use requires frequent monitoring for toxicity, ultimately causing 20% of patients to discontinue therapy. The identification of more effective and better tolerated agents is therefore desirable.

The synthesis of chemical libraries rich in structural diversity is an emerging field with immediate applications in biomedical research. Strategies for library synthesis have mainly focused on combinatorial synthesis of molecules that vary substituents on a core structural type. Diversity-oriented synthesis (DOS) is a new strategy for constructing chemical libraries with both skeletal and functional group diversity, and, in combination with phenotype-based assays, has emerged as a powerful tool for the identification of potential therapeutic agents and/or biological probes. The goal of diversity-oriented synthesis is the facile preparation of collections of structurally complex, and preferably biologically-active, compounds from simple starting materials, typically through the use of “split-pool” combinatorial chemistry. The generation of DOS libraries, such as, for example, the SpOx library (Lo et al., J. Am. Chem. Soc. (2004) 126:16077-16086), the DHPC library (Stavenger et al., Angew. Chem. Int. Ed. Engl. (2001) 40:3417-3421), and the FOLD library (Burke et al., J. Am. Chem. Soc. (2004) 126:14095-14104), have become an effective means of advancing drug discovery.

### SUMMARY OF THE INVENTION

The present invention provides novel compounds and pharmaceutically acceptable forms thereof; pharmaceutical compositions; and methods of treating a viral infection, such as a hepatitis C virus (HCV) infection, by administering to a subject diagnosed with being susceptible to a viral infection a therapeutically effective amount of an inventive compound or pharmaceutical composition thereof.

The present invention also provides a high-throughput screening system of identifying compounds capable of modulating HCV replication.

In one aspect, the present invention provides a compound, or a pharmaceutically acceptable form thereof, having the formula (I):

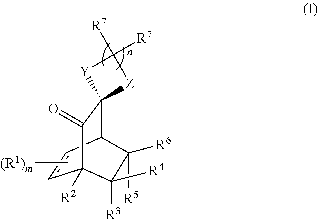

wherein

each instance of R¹ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl;

R², R³, R⁴, R⁵, and R⁶, are, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;

or R¹ and R² together form a 5- to 6-membered, substituted or unsubstituted, carbocyclic or heterocyclic ring; R² and R³ together form a 5- to 6-membered, substituted or unsubstituted, carbocyclic or heterocyclic ring; R³ and R⁴ together form a 5- to 6-membered, substituted or unsubstituted, carbocyclic or heterocyclic ring; R³ and R⁵ together form a 5- to 6-membered, substituted or unsubstituted, carbocyclic or heterocyclic ring; R⁵ and R⁶ together form a 5- to 6-membered, substituted or unsubstituted, carbocyclic or heterocyclic ring; or R⁴ and R⁶ together form a 5- to 6-membered, substituted or unsubstituted, carbocyclic or heterocyclic ring;

each instance of R⁷ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl; or two R⁷ groups taken together form an oxo (═O), an imino (═NH), or a thiooxo (═S) group;

Y and Z are, independently, —O—, —S—, N(R^{C})—, or —C(R^{C})₂—, wherein each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or two R^{C} groups taken together form an (═O), an (═NH), or an (═S) group;

[symbol] corresponds to a single or double bond;

m is 0, 1, 2, 3, or 4; and

n is 0, 1, 2, or 3.

In another aspect, the present invention provides a compound, or a pharmaceutically acceptable form thereof, having the formula:

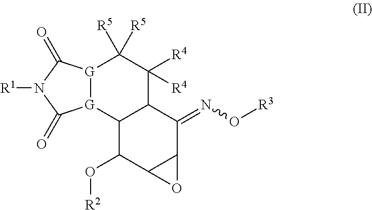

wherein

each instance of G is, independently, —N— or —(CH)—;

R¹ is hydrogen; hydroxyl; halogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable amino protecting group;

R² is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable hydroxyl protecting group;

R³ is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable hydroxyl protecting group; and

each instance of R⁴ is, independently, hydrogen; halogen; substituted or unsubstituted hydroxyl; substituted or unsubstituted amino; substituted or unsubstituted thiol; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or substituted or unsubstituted acyl; or two R⁴ groups taken together form an oxo (═O), an imino (═NH), or a thiooxo (═S) group; and

each instance of R⁵ is, independently, hydrogen; hydrogen; halogen; substituted or unsubstituted hydroxyl; substituted or unsubstituted amino; substituted or unsubstituted thiol; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or substituted or unsubstituted acyl; or two R⁵ groups taken together form an oxo (═O), an imino (═NH), or a thiooxo (═S) group.

In a third aspect, the present invention provides pharmaceutical compositions comprising an inventive compound of any of the above formulae (e.g., (I) or (II)), and a pharmaceutically acceptable excipient.

In a fourth aspect, the present invention provides methods of treating a viral infection comprising administering to a subject diagnosed with a viral infection, or being susceptible to infection by a virus, a therapeutically effective amount of an inventive compound, or a pharmaceutical composition thereof.

In a fifth aspect, the present invention provides a high-throughput method of identifying compounds able to modulate HCV replication comprising the steps of: providing a multiwell plate comprising at least about 90 wells per plate; adding at least one cell with a Huh7/Rep-Feo HCV replicon to each of the wells; providing at least one test compound; contacting the test compound to the cell under suitable conditions to illicit a change in HCV RNA replication; and detecting a change in luciferase activity, wherein luciferase activity is functionally linked to HCV RNA replication activity, and a change in luciferase activity is directly proportional to a change in HCV replication activity. In certain embodiments, the above method further comprises providing a second multiwell plate comprising at least about 10 wells per plate; adding at least one HCV infected cell; providing at least 1 test compound; contacting the compound to the cell; and assessing the cytotoxicity of the test compound to the HCV infected cell of step (vii).

This application refers to various issued patents, published patent applications, journal articles, and other publications, all of which are incorporated herein by reference.

The details of one or more embodiments of the invention are set forth in the accompanying Figures and the Detailed Description of Certain Embodiments, as described below. Other features, objects, and advantages of the invention will be apparent from the description, the figures, and from the claims.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

It will be appreciated that the compounds of the present invention, as described herein, may be substituted with any number of substituents or functional moieties. In general, the term “substituted” whether preeceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like) that results in the formation of a stable moiety. The present invention contemplates any and all such combinations in order to arrive at a stable substituent/moiety. For purposes of this invention, heteroatoms such as nitrogen may have hydrogen substituents and/or any suitable substituent as described herein which satisfy the valencies of the heteroatoms and results in the formation of a stable moiety.

The term “acyl,” as used herein, refers to a group having the general formula —C(═O)R^{A}, —C(═O)OR^{A}, —C(═O)—O—C(═O)R^{A}, —C(═O)SR^{A}, —C(═O)N(R^{A})₂, —C(═S)R^{A}, —C(═S)N(R^{A})₂, and —C(═S)S(R^{A}), —C(═NR^{A})R^{A}, —C(═NR^{A})OR^{A}, —C(═NR^{A})SR^{A}, and —C(═NR^{A})N(R^{A})₂, wherein R^{A} is hydrogen; halogen; optionally substituted hydroxyl; optionally substituted thiol; optionally substituted amino; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{A} groups taken together form a 5-to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

An “activated carboxylic acid,” as used herein, includes esters (e.g., —C(═O)OR^{A}), anhydrides (e.g., —C(═O)—O—C(═O)R^{A}), acyl halides (e.g., —C(═O)Br, —C(═O)Cl, —C(═O)I), sulfonylated carboxylic acids (e.g., —C(O)O-trifluoromethylsulfonyl (—OTf), —C(O)O-tolylsulfonyl (—OTs), —C(O)O-methanesulfonyl (—OMs), —C(O)O-(4-nitrophenylsulfonyl) (—ONos), and —C(O)O-(2-nitrophenylsulfonyl) (—ONs)), and the like, and wherein each instance of R^{A} is, independently, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl.

An “activated” hydroxyl group,” as used herein, includes sulfonyl groups (e.g., O-trifluoromethylsulfonyl (—OTf), O-tolylsulfonyl (—OTs), O-methanesulfonyl (—OMs), O-(4-nitrophenylsulfonyl) (—ONos), and O-(2-nitrophenylsulfonyl) (—ONs)), and acyl groups.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-12 carbon atoms. In still other embodiments, the alkyl group contains 1-6 carbon atoms. In yet another embodiments, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substitutents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-10 carbon atoms. In another embodiment, the alkenyl group employed contains 2-8 carbon atoms. In still other embodiments, the alkenyl group contains 2-6 carbon atoms. In yet another embodiments, the alkenyl group contains 2-4 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-10 carbon atoms. In another embodiment, the alkynyl group employed contains 2-8 carbon atoms. In still other embodiments, the alkynyl group contains 2-6 carbon atoms. In other embodiments, the alkynyl group contains 2-4 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “amino,” as used herein, refers to a group of the formula (—NH₂). An “substituted amino” refers to a mono-substituted amino group of the formula (—NHR^{h}) and a di-substituted amino group of the formula (—NR^{h}₂). Substituents include, but are not limited to, any of the substituents described herein, that result in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted). In certain embodiments, the R^{h} substituents of the di-substituted amino group(—NR^{h}₂) form a 5- to 6-membered heterocyclic ring.

The term “aliphaticamino,” refers to a “substituted amino” of the formula (—NR^{h}₂), wherein R^{h} is, independently, a hydrogen or an optionally substituted aliphatic group, as defined herein, and the amino moiety is directly attached to the parent molecule.

The term “aliphaticoxy,” refers to a “substituted hydroxyl” of the formula (—ORⁱ), wherein Rⁱ is an optionally substituted aliphatic group, as defined herein, and the oxygen moiety is directly attached to the parent molecule.

The term “alkyloxy” refers to a “substituted hydroxyl” of the formula (—ORⁱ), wherein Rⁱ is an optionally substituted alkyl group, as defined herein, and the oxygen moiety is directly attached to the parent molecule.

The term “alkylthioxy” refers to a “substituted thiol” of the formula (—SR^{r}), wherein R^{r} is an optionally substituted alkyl group, as defined herein, and the sulfur moiety is directly attached to the parent molecule.

The term “alkylamino” refers to a “substituted amino” of the formula (—NR^{h}₂), wherein R^{h} is, independently, a hydrogen or an optionally substituted alkyl group, as defined herein, and the nitrogen moiety is directly attached to the parent molecule.

The term “aryl,” as used herein, refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “arylalkyl,” as used herein, refers to an aryl substituted alkyl group, wherein the terms “aryl” and “alkyl” are defined herein, and wherein the aryl group is attached to the alkyl group, which in turn is attached to the parent molecule. An exemplary arylalkyl group includes benzyl.

The term “aryloxy” refers to a “substituted hydroxyl” of the formula (—ORⁱ), wherein Rⁱ is an optionally substituted aryl group, as defined herein, and the oxygen moiety is directly attached to the parent molecule.

The term “arylamino,” refers to a “substituted amino” of the formula (—NR^{h}₂), wherein R^{h} is, independently, a hydrogen or an optionally substituted aryl group, as defined herein, and the nitrogen moiety is directly attached to the parent molecule.

The term “arylthioxy” refers to a “substituted thiol” of the formula (—SR^{r}), wherein R^{r} is an optionally substituted aryl group, as defined herein, and the sulfur moiety is directly attached to the parent molecule.

The term “azido,” as used herein, refers to a group of the formula (—N₃). An “optionally substituted azido” refers to a group of the formula (—N₃R^{t}), wherein R^{t} can be any substitutent (other than hydrogen). Substituents include, but are not limited to, any of the substituents described herein, that result in the formation of a stable moiety (e.g., a suitable amino protecting group; aliphatic, alkyl, alkenyl, alkynyl, heteroaliphatic, heterocyclic, aryl, heteroaryl, acyl, and the like, each of which may or may not be further substituted).

The term “carbocyclic,” “carbocycles,” or “carbocyclyl,” as used herein, refers to a cyclic aliphatic group. A carbocyclic group refers to an all-carbon, non-aromatic, partially unsaturated or fully saturated, 3- to 10-membered ring system, which includes single rings of 3 to 8 atoms in size and bi- and tri-cyclic ring systems, and which may include aromatic five- or six-membered aryl groups fused to the carbocyclic ring. Exemplary carbocycles include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, cyclooctenyl, cyclononenyl, cyclodecenyl, and the like, and which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, as used herein, the term “heteroalkyl” includes straight, branched and cyclic alkyl groups that contain one or more oxygen, sulfur, nitrogen, phosphorus, or silicon atoms, e.g., in place of carbon atoms. An analogous convention applies to other generic terms such as “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “heterocyclic,” “heterocycles,” or “heterocyclyl,” as used herein, refers to a cyclic heteroaliphatic. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, and the like, each of which may or may not be further substituted).

The term “heteroaliphaticamino” refers to a “substituted amino” of the formula (—NR^{h}₂), wherein R^{h} is, independently, a hydrogen or an optionally substituted heteroaliphatic group, as defined herein, and the nitrogen moiety is directly attached to the parent molecule.

The term “heteroaliphaticoxy” refers to a “substituted hydroxyl” of the formula (—ORⁱ), wherein Rⁱ is an optionally substituted heteroaliphatic group, as defined herein, and the oxygen moiety is directly attached to the parent molecule.

The term “heteroaliphaticthioxy” refers to a “substituted thiol” of the formula (—SR^{r}), wherein R^{r} is an optionally substituted heteroaliphatic group, as defined herein, and the sulfur moiety is directly attached to the parent molecule.

The term “heteroalkylamino” refers to a “substituted amino” of the formula (—NR^{h}₂), wherein R^{h} is, independently, a hydrogen or an optionally substituted heteroalkyl group, as defined herein, and the nitrogen moiety is directly attached to the parent molecule.

The term “heteroalkyloxy” refers to a “substituted hydroxyl” of the formula (—ORⁱ), wherein Rⁱ is an optionally substituted heteroalkyl group, as defined herein, and the oxygen moiety is directly attached to the parent molecule.

The term “heteroalkylthioxy” refers to a “substituted thiol” of the formula (—SR^{r}), wherein R^{r} is an optionally substituted heteroalkyl group, as defined herein, and the sulfur moiety is directly attached to the parent molecule.

The term “heteroarylamino” refers to a “substituted amino” of the (—NR^{h}₂), wherein R^{h} is, independently, a hydrogen or an optionally substituted heteroaryl group, as defined herein, and the nitrogen moiety is directly attached to the parent molecule.

The term “heteroaryloxy” refers to a “substituted hydroxyl” of the formula (—ORⁱ), wherein Rⁱ is an optionally substituted heteroaryl group, as defined herein, and the oxygen moiety is directly attached to the parent molecule.

The term “heteroarylthioxy” refers to a “substituted thiol” of the formula (—SR^{r}), wherein R^{r} is an optionally substituted heteroaryl group, as defined herein, and the sulfur moiety is directly attached to the parent molecule.

The term “hydroxy,” or “hydroxyl,” as used herein, refers to a group of the formula (—OH). An “optionally substituted hydroxyl” refers to a group of the formula (—ORⁱ), wherein Rⁱ can be hydrogen, or any substitutent which results in a stable moiety (e.g., a suitable hydroxyl protecting group; aliphatic, alkyl, alkenyl, alkynyl, heteroaliphatic, heterocyclic, aryl, heteroaryl, acyl, sulfinyl, sulfonyl, nitro, alkylaryl, arylalkyl, and the like, each of which may or may not be further substituted). In certain embodiments, an optionally substituted hydroxyl includes an “alkyloxy” or a “heteroalkyloxy” group.

The term “imino,” as used herein, refers to a group of the formula (═NR^{r}), wherein R^{r} corresponds to hydrogen or any substitutent as described herein, that results in the formation of a stable moiety (for example, a suitable amino protecting group; aliphatic, alkyl, alkenyl, alkynyl, heteroaliphatic, heterocyclic, aryl, heteroaryl, acyl, sulfinyl, sulfonyl, amino, hydroxyl, alkylaryl, arylalkyl, and the like, each of which may or may not be further substituted).

The term “isocyano,” as used herein, refers to a group of the formula (—NC).

The term “nitro,” as used herein, refers to a group of the formula (—NO₂).

The term “oxo,” as used herein, refers to a group of the formula (═O).

The term “stable moiety,” as used herein, preferably refers to a moiety which possess stability sufficient to allow manufacture, and which maintains its integrity for a sufficient period of time to be useful for the purposes detailed herein.

A “suitable amino-protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “suitable carboxylic acid protecting group,” or “protected carboxylic acid,” as used herein, are well known in the art and include those described in detail in Greene (1999). Examples of suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

A “suitable hydroxyl protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “suitable thiol protecting group,” as used herein, are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of suitable ester groups include formates, acetates, proprionates, pentanoates, crotonates, and benzoates. Specific examples of suitable ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl groups include trimethylsilyl, triethylsilyl, t-butyldimethylsilyl, t-butyldiphenylsilyl, triisopropylsilyl ether, and other trialkylsilyl ethers. Examples of suitable alkyl groups include methyl, benzyl, p-methoxybenzyl, 3,4-dimethoxybenzyl, trityl, t-butyl, and allyl ether, or derivatives thereof. Examples of suitable arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The term “thio,” or “thiol,” as used herein, refers to a group of the formula (—SH). An “optionally substituted thiol” refers to a group of the formula (—SR^{r}), wherein R^{r} can be hydrogen, or any substitutent. Substituents include, but are not limited to, any of the substituents described herein, that result in the formation of a stable moiety (e.g., a suitable thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “thiooxo,” as used herein, refers to a group of the formula (═S).

Additional examples of generally applicable substitutents are illustrated by the specific embodiments shown in the Examples, which are described herein.

As used herein, a “pharmaceutically acceptable form thereof” includes any pharmaceutically acceptable salts, prodrugs, tautomers, isomers, and/or polymorphs of a compound of the present invention, as defined below and herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “prodrug” refers to a derivative of a parent compound that requires transformation within the body in order to release the parent compound. In certain cases, a prodrug has improved physical and/or delivery properties over the parent compound. Prodrugs are typically designed to enhance pharmaceutically and/or pharmacokinetically based properties associated with the parent compound. The advantage of a prodrug can lie in its physical properties, such as enhanced water solubility for parenteral administration at physiological pH compared to the parent compound, or it enhances absorption from the digestive tract, or it may enhance drug stability for long-term storage. In recent years several types of bioreversible derivatives have been exploited for utilization in designing prodrugs. Using esters as a prodrug type for compounds containing a carboxyl or hydroxyl functionality is known in the art as described, for example, in “The Organic Chemistry of Drug Design and Drug Interaction” Richard Silverman, published by Academic Press (1992).

As used herein, the term “tautomer” includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol; amide-to-imide; lactam-to-lactim; enamine-to-imine; and enamine-to-(a different) enamine tautomerizations.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

As used herein, “polymorph” refers to a crystalline inventive compound existing in more than one crystalline form/structure. When polymorphism exists as a result of difference in crystal packing it is called packing polymorphism. Polymorphism can also result from the existence of different conformers of the same molecule in conformational polymorphism. In pseudopolymorphism the different crystal types are the result of hydration or solvation.

As used herein, the term “labeled” is intended to mean that a compound of the present invention that has at least one element, isotope, or chemical compound attached to enable the detection of the compound. In general, labels typically fall into three classes: a) isotopic labels, which may be radioactive or heavy isotopes, including, but not limited to, ²H, ³H, ³²P, ³⁵S, ⁶⁷Ga, ^{99m}Tc (Tc-99m), ¹¹¹In, ¹²³In, ¹²⁵I, ¹⁶⁹Yb and ¹⁸⁶Re; b) immune labels, which may be antibodies or antigens, which may be bound to enzymes (such as horseradish peroxidase) that produce detectable agents; and c) colored, luminescent, phosphorescent, or fluorescent dyes (e.g., photoaffinity labeling, see, for example, Bayley, H., Photogenerated Reagents in Biochemistry and Molecular Biology (1983), Elsevier, Amsterdam, the entire contents of which are hereby incorporated by reference). It will be appreciated that the labels may be incorporated at any position that does not interfere with the activity of the compound.

As used herein, to “modulate” or “modulating” refers to a change, such as an increase, decrease, or inhibition, of activity.

### The Following Definitions are More General Terms Used Throughout the Present Application:

The term “subject,” as used herein, refers to any animal. In certain embodiments, the subject is a mammal. In certain embodiments, the term “subject”, as used herein, refers to a human (e.g., a man, a woman, an adolescent, a child).

The terms “treat” or “treating,” as used herein, refers to partially or completely preventing, ameliorating, reducing, delaying, or diminishing the severity of a viral infection or symptoms related to a viral infection from which the subject is suffering.

The terms “administer,” “administering,” or “administration,” as used herein refers to implanting, absorbing, ingesting, injecting, or inhaling, an inventive compound(s) or pharmaceutical composition. In certain embodiments, one or more compounds of the present invention, or an inventive pharmaceutical composition, is administered to the subject by any known means; for example, administration may be made transdermally, orally, parenterally, intravenously (IV), intraarterially, by implantion, by absorbtion from the eyes, skin, nasal passages, rectum, vagina, etc., by ingestion, by injection, and/or by inhalation.

The terms “effective amount” and “therapeutically effective amount,” as used herein, refer to the amount or concentration of an inventive compound or inventive pharmaceutical composition that, when administered to a subject, is effective to at least partially treat a condition from which the subject is suffering. As used herein, an “effective amount” or “therapeutically effective amount” of an inventive compound or inventive pharmaceutical composition is an amount that can achieve a desired therapeutic and/or prophylactic effect. A “therapeutically effective amount” is at least a minimal amount of an inventive compound or inventive pharmaceutical composition which is sufficient for preventing, ameliorating, reducing, delaying, or diminishing the severity of a viral infection, or symptoms related to a viral infection, from which a subject is suffering.

The expression “unit dosage form,” as used herein, refers to a physically discrete unit of inventive pharmaceutical composition/formulation, as described herein, appropriate for the subject to be treated. In general, a unit dosage form of an inventive pharmaceutical composition/formulation is a discrete physical entity intended for delivery, typically orally, to a subject. A unit dosage form may or may not constitute a single “dose” of a compound of compounds present in the composition of the present invention, as a prescribing doctor may choose to administer more than one, less than one, or precisely one unit dosage form in each dose (i.e., each instance of administration). For example, unit dosage forms may be administered once or more than once a day, for example, 2, 3 or 4 times a day. It will be understood, however, that the total daily usage of the pharmaceutical compositions of the present invention will be decided by the attending physician within the scope of sound medical judgment. The specific effective dose level for any particular subject will depend upon a variety of factors including the infection being treated and the severity of the infection; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

A “therapeutically active agent” or “biologically active agent” or “active agent” refers to a biologically active substance, that is useful for therapy (e.g., human therapy, veterinary therapy), including prophylactic and therapeutic treatment. Therapeutically active agents include organic molecules that are drug compounds, peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, and vitamins. Therapeutically active agents include any substance used as a medicine for treatment, prevention, delay, reduction or amelioration of a disease, condition, or disorder, and refers to a substance that is useful for therapy, including prophylactic and therapeutic treatment. A therapeutically active agent also includes a compound that increases the effect or effectiveness of another compound, for example, by enhancing potency or reducing adverse effects of the other compound. In certain embodiments, the pharmaceutical compositions include any number of additional therapeutically active agents, for example, a second, third, fourth, or fifth, therapeutically active agent.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction is when two entities are covalently connected through a linker group.

### BRIEF DESCRIPTION OF THE FIGURES

FIG. 1. A graphical summary of the primary screening of the DOS set using the Huh7/Rep-Feo replicon cell line. Each point represents one compound. The X-axis shows HCV replication as measured by normalized luciferase signal, expressed as the composite Z-score. The Y-axis shows cell viability as measured by normalized CellTiterGlo (Promega) signal, expressed as the composite Z-score.

FIG. 2. Scheme for syntheses of SM_A14B5 and SM_A12B3. Briefly, periodate oxidation of the alcohol substrate leads to formation of a spiro bicyclic diene (with an epoxide in SM_A14B5 and a tetrahydrofuran in SM_A12B3) in situ. The diene then undergoes a [4+2] cycloaddition with the appropriate dienophile (styrene for SM_A14B5 and a substituted butanediol ether for SM_A12B3) to give the desired compounds. BTEAC=benzyltriethylammonium chloride, BTIB=I,I-Bis(trifluoroacetoxy)iodo]benzene.

FIG. 3. General scheme for the synthesis of BUCMLD compounds, as described in Lei et al., J. Org. Chem. (2005) 70:6474-6483.

FIG. 4. Results of secondary screening with antiviral hit compounds from the SM library. Luciferase activity for HCV RNA replication levels is shown as a percentage of control. On those graphs with two curves, cell viability is also shown as a percentage of control. Each point represents the average of triplicate data points with standard deviation represented as the error bar.

FIG. 5. Results of secondary screening with custom synthesized compounds derived from the SM library. Luciferase activity for HCV RNA replication levels is shown as a percentage of control. Cell viability is also shown as a percentage of control. Each point represents the average of triplicate data points with standard deviation represented as the error bar.

FIGS. 6 to 8. Results of secondary screening with antiviral hit compounds from the BUCMLD library. Luciferase activity for HCV RNA replication levels is shown as a percentage of control. For the lead compounds (BUCMLD-B10A11, BUCMLD-B10A3, and BUCMLD-B10A5), there are second graphs over a more detailed range of concentrations, wherein both luciferase activity for HCV RNA replication levels (reporter) and cell viability are shown as a percentage of control. Each point represents the average of triplicate data points with standard deviation represented as the error bar.

FIG. 9. Luciferase activity of different cell inoculation concentrations in the 384-well plate format. Different concentrations of PEG-IFN were used as a positive control and to determine signal-to-background (S/B) ratio. HCV RNA replication levels were determined by luciferase activity. Each point represents the average of 3 data points, with the standard deviation represented as data bars.

FIG. 10. Hits from the primary HTS with the known bioactives library. There are 21 antiviral compounds that inhibited HCV replication and 28 proviral compounds that increased HCV replication. R-CompZ represents the compositeZ score for the luciferase reporter gene assay. C-CompZ represents the compositeZ score for the cell viability assay.

FIG. 11. A graphical summary of the primary screening for the known bioactives library (as listed in FIG. 10) using Huh7/Rep-Feo cell. Each point represents 1 compound. The X-axis shows HCV replication as measured by normalized luciferase signal, expressed as the composite Z-score. The Y-axis shows cell viability as measured by normalized CellTiterGlo (Promega) signal, expressed as the composite Z-score.

FIGS. 12A and 12B. (A) Anti-HCV activity of PEG-IFN and ribavirin on HCV RNA replication in OR6 cell system. OR6 cells were cotreated with PEG-IFN (0, 0.001, 0.002, 0.007, 0.03, and 0.07 ng/mL) and ribavirin (0, 25, 50, 100, 200, and 400 μM) for 48 hours. Luciferase activity for HCV RNA replication levels is shown as a percentage of control. Each bar represents the average of triplicate data points with standard deviation represented as the error bar. (B) A normalized isobologram generated by CalcuSyn using the data from FIG. 12A. Points below and to the left of the line represent synergy. Thirteen of the fourteen concentration ratios examined demonstrate the synergistic effect of the combination of PEGIFN and ribavirin.

FIGS. 13A and 13B. Results of secondary screening with corticosteroids. (A) Luciferase activity for HCV RNA replication levels is shown as a percentage of control. (B) Cell viability is also shown as a percentage of control. Each bar represents the average of triplicate data points with standard deviation represented as the error bar. *Denotes a significant difference from control of at least P<0.05.

FIGS. 14A to 14F. Results of secondary screening with several hit compounds. Luciferase activity for HCV RNA replication levels is shown as a percentage of control (A, C, E). Cell viability is also shown as a percentage of control (B, D, F). Each bar represents the average of triplicate data points with standard deviation represented as the error bar. (A, B) MY-5445 and trequinsin (C, D) SB 203580 (E, F) tetrandrine. *Denotes a significant difference from control of at least P<0.05.

FIGS. 15A and 15B. Results of secondary screening with the statins. (A) Luciferase activity for HCV RNA replication levels is shown as a percentage of control. (B) Cell viability is shown as a percentage of control. Each bar represents the average of triplicate data points with standard deviation represented as the error bar. *Denotes significant difference from control of at least P<0.05.

### DETAILED DESCRIPTION OF CERTAIN EMBODIMENTS OF THE INVENTION

The present invention provides compounds for the treatment and prevention of HCV. The inventive compounds may be used in pharmaceutical compositions to treat or prevent HCV infection. Therapeutically effective amounts of an inventive compound or pharmaceutical composition thereof are administered to a subject to treat or prevent a viral (e.g., HCV infection). The present invention also provides a high-throughput system of identifying compounds capable of modulating HCV replication. Such a high-throughput system includes methods, materials, and kits.

In one aspect, the present invention provides an inventive compound, or a pharmaceutically acceptable form thereof, having the formula (I):

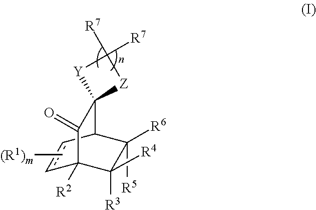

wherein

each instance of R¹ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;

R², R³, R⁴, R⁵, and R⁶, are, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;

or R¹ and R² together form a 5- to 6-membered, substituted or unsubstituted, carbocyclic or heterocyclic ring; R² and R³ together form a 5- to 6-membered, substituted or unsubstituted, carbocyclic or heterocyclic ring; R³ and R⁴ together form a 5- to 6-membered, substituted or unsubstituted, carbocyclic or heterocyclic ring; R³ and R⁵ together form a 5- to 6-membered, substituted or unsubstituted, carbocyclic or heterocyclic ring; R⁵ and R⁶ together form a 5- to 6-membered, substituted or unsubstituted, carbocyclic or heterocyclic ring; or R⁴ and R⁶ together form a 5- to 6-membered, substituted or unsubstituted, carbocyclic or heterocyclic ring;

each instance of R⁷ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl; substituted or unsubstituted acyl; or two R⁷ groups taken together form an oxo (═O), an imino (═NH), or a thiooxo (═S) group;

Y and Z are, independently, —O—, —S—, —N(R^{C})—, or —C(R^{C})₂—, wherein each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or two R^{C} groups taken together form an oxo (═O), an imino (═NH), or a thiooxo (═S) group;

[symbol] corresponds to a single or double bond;

m is 0, 1, 2, 3, or 4; and

n is 0, 1, 2, or 3.

In certain embodiments, each instance of R¹ is, independently, hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl.

In certain embodiments, each instance of R¹ is, independently, hydrogen; halogen; cyclic or acyclic, substituted or unsubstituted, branched or unbranched aliphatic; substituted or unsubstituted aryl; or —C(═O)OR^{A}, wherein each instance of R^{A} is, independently, cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

In certain embodiments, R¹ is, hydrogen, halogen (i.e., iodo, bromo, chloro and fluoro), or cyclic or acyclic, substituted or unsubstituted, branched or unbranched aliphatic. In certain embodiments, R¹ is hydrogen, halogen, or cyclic or acyclic, substituted or unsubstituted, branched or unbranched alkyl. In certain embodiments, R¹ is hydrogen, halogen, or cyclic or acyclic, substituted or unsubstituted, branched or unbranched C₁₋₁₂ alkyl. In certain embodiments, R¹ is hydrogen, halogen, or cyclic or acyclic, substituted or unsubstituted, branched or unbranched C₁₋₆ alkyl. In certain embodiments, R¹ is hydrogen, halogen, or cyclic or acyclic, substituted or unsubstituted, branched or unbranched C₁₋₄ alkyl.

In certain embodiments, R¹ is hydrogen or halogen. In certain embodiments, R¹ is hydrogen or iodo (—I). In certain embodiments, R¹ is hydrogen or bromo (—Br). In certain embodiments, R¹ is hydrogen or chloro (—Cl). In certain embodiments, R¹ is hydrogen or fluoro (—F).

In certain embodiments, R² is hydrogen; halogen; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl.

In certain embodiments, R² is hydrogen; halogen; substituted or unsubstituted hydroxyl; cyclic or acyclic, substituted or unsubstituted, branched or unbranched aliphatic; cyclic or acyclic, substituted or unsubstituted, branched or unbranched heteroaliphatic; or substituted or unsubstituted acyl.

In certain embodiments, R² is hydrogen; halogen; —WR^{D}; —CH₂WR^{D}; —CH₂CH₂WR^{D}; —CH₂CH₂CH₂WR^{D}; or —CH₂CH₂CH₂CH₂WR^{D}, wherein W is —O—, —S—, or —N(R^{W})—; R^{W} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or a suitable amino protecting group; and R^{D} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or a suitable hydroxyl protecting group, a suitable thiol protecting group, or a suitable amino protecting group, or R^{D} and R^{W} taken together form a 5- to 6-membered heterocyclic ring.

In certain embodiments, W is —O—. In certain embodiments, W is —O—, and R^{D} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or a suitable hydroxyl protecting group.

In certain embodiments, W is —S—. In certain embodiments, W is —S—, and R^{D} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or a suitable thiol protecting group.

In certain embodiments, W is —N(R^{w})—. In certain embodiments, W is —N(R^{w})—, and R^{D} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; substituted or unsubstituted acyl; or a suitable amino protecting group.

In certain embodiments, both R^{D} and R^{W} are hydrogen.

In certain embodiments, R³ is hydrogen; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl.

In certain embodiments, R³ is substituted or unsubstituted aryl; substituted or unsubstituted heteroaryl; cyclic or acyclic, substituted or unsubstituted aliphaticoxy; or cyclic or acyclic, substituted or unsubstituted heteroaliphaticoxy. In certain embodiments, R³ is substituted or unsubstituted aryl; substituted or unsubstituted heteroaryl; cyclic or acyclic, substituted or unsubstituted alkyloxy; or cyclic or acyclic, substituted or unsubstituted heteroalkyloxy.

In certain embodiments, R³ corresponds to the formula:

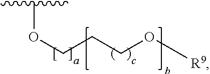

wherein

R⁹ is hydrogen; substituted or unsubstituted aliphatic; substituted or unsubstituted aryl; substituted or unsubstituted heteroaryl; substituted or unsubstituted acyl; or a suitable hydroxyl protecting group;

a is 0, 1, 2, 3, 4, 5, or 6;

b is 0, 1, 2, or 3; and

c is 1, 2, 3, 4, 5, or 6.

In certain embodiments, a is 0. In certain embodiments, a is 1. In certain embodiments, a is 2. In certain embodiments, a is 3. In certain embodiments, a is 4. In certain embodiments, a is 5. In certain embodiments, a is 6. In certain embodiments, a is 2 to 6.

In certain embodiments, b is 0. In certain embodiments, b is 1. In certain embodiments, b is 2. In certain embodiments, b is 3. In certain embodiments, b is 1 to 3.

In certain embodiments, c is 0. In certain embodiments, c is 1. In certain embodiments, c is 2. In certain embodiments, c is 3. In certain embodiments, c is 4. In certain embodiments, c is 5. In certain embodiments, c is 6. In certain embodiments, c is 1 to 6.

In certain embodiments, R³ corresponds to the formula:

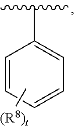

wherein

R⁸ is halogen; substituted or unsubstituted hydroxyl; substituted or unsubstituted thiol; substituted or unsubstituted amino; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; nitro; cyano; isocyano; azido; substituted or unsubstituted acyl, —SO₃R^{E}; —SO₂R^{E}, or —SOR^{E}, wherein R^{E} is hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl; and

and

t is 0, 1, 2, 3, 4, or 5.

In certain embodiments, R³ corresponds to the formulae:

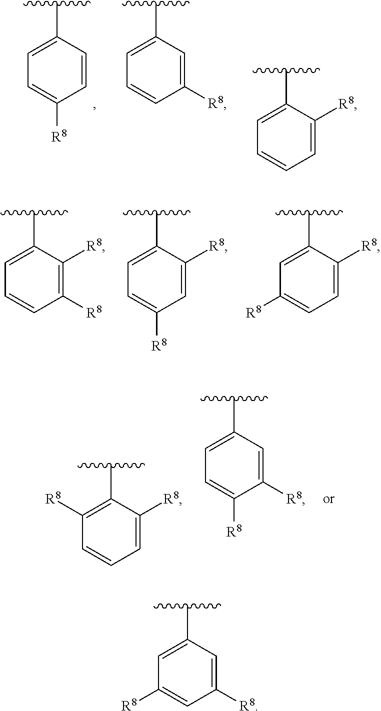

In certain embodiments, R⁸ is halogen; substituted or unsubstituted hydroxyl; or cyclic or acyclic, substituted or unsubstituted, branched or unbranched aliphatic.

In certain embodiments, R² and R³ together form a 5- to 6-membered, substituted or unsubstituted, heterocyclic ring. In certain embodiments, R² and R³ together form a 5-membered, substituted or unsubstituted, heterocyclic ring. In certain embodiments, R² and R³ together form a 5-membered, unsubstituted, heterocyclic ring. In certain embodiments, In certain embodiments, R² and R³ together form a 6-membered, substituted or unsubstituted, heterocyclic ring. In certain embodiments, R² and R³ together form a 6-membered, unsubstituted, heterocyclic ring. In certain embodiments, R² and R³ together form either a tetrahydrofuran or a tetrahydropyran ring.

In certain embodiments, R⁴ and R⁶ together form a 5- to 6-membered, substituted or unsubstituted, carbocyclic or heterocyclic ring. In certain embodiments, R⁴ and R⁶ together form a 5-membered, substituted or unsubstituted, carbocyclic ring. In certain embodiments, R⁴ and R⁶ together form a 6-membered, substituted or unsubstituted, carbocyclic ring.

In certain embodiments, each instance of R⁷ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

In certain embodiments, each instance of R⁷ is, independently, hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

In certain embodiments, Y and Z are, independently, —O—, —S—, —N(R^{C})—, or —C(R^{C})₂—, wherein each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or two R^{C} groups taken together form an (═O), an (═NH), or an (═S) group.

In certain embodiments, Y is —O—, —S—, or —N(R^{C})—, and Z is —C(R^{C})₂—. In certain embodiments, Z is —O—, —S—, or —N(R^{C})—, and Y is —C(R^{C})₂—. However, in certain embodiments, Y is —O—, —S—, or —N(R^{C})—, and Z is —O—, —S—, or —N(R^{C})—. In certain embodiments, both Y and Z are —O—.

In certain embodiments, each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

In certain embodiments, each instance of R^{C} is, independently, hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

In certain embodiments, [symbol] corresponds to a single bond, and m is 0, 1, 2, 3, or 4. In certain embodiments, [symbol] corresponds to a double bond, and m is 0, 1, or 2.

In certain embodiments, n is 0, 1, or 2. In certain embodiments, n is 0.

In certain embodiments, R⁴ is hydrogen. In certain embodiments, R⁵ is hydrogen. In certain embodiments, R⁶ is hydrogen. In certain embodiments, R^{C} is hydrogen. In certain embodiments, each of R⁴, R⁵, R⁶, and R^{C} are hydrogen.

In certain embodiments, the inventive compound of formula (I), or a pharmaceutically acceptable form thereof, corresponds to the formula (I-a):

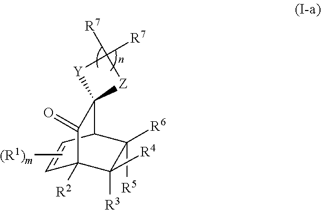

wherein

R¹, R², R³, R⁴, R⁵, R⁶, R⁷, Y, Z, and n are as defined above and herein; and

m is 0, 1, or 2.

In certain embodiments, the inventive compound of formula (I), or a pharmaceutically acceptable form thereof, corresponds to the formula (I-b):

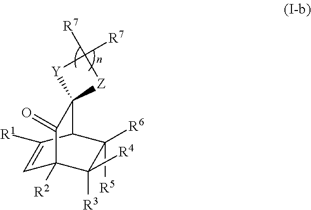

wherein

R¹, R², R³, R⁴, R⁵, R⁶, R⁷, Y, Z, and n are as defined above and herein.

In certain embodiments, the inventive compound of formula (I), or a pharmaceutically acceptable form thereof, corresponds to the formulae (I-c) or (I-c′):

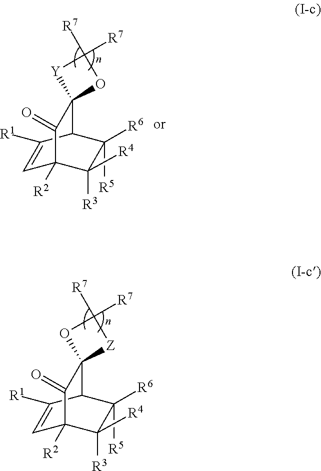

wherein

R¹, R², R³, R⁴, R⁵, R⁶, R⁷, Y, Z, and n are as defined above and herein.

In certain embodiments, the inventive compound of formula (I), or a pharmaceutically acceptable form thereof, corresponds to the formulae (I-d) or (I-d′):

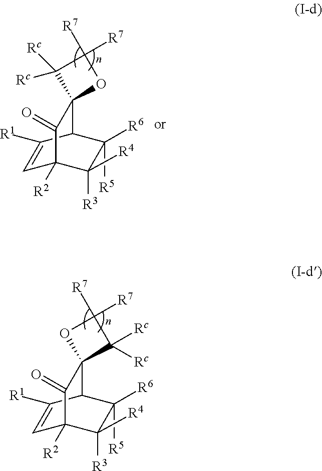

wherein

R′, R², R³, R⁴, R⁵, R⁶, R⁷, R^{C}, and n are as defined above and herein.

In certain embodiments, the inventive compound of formula (I), or a pharmaceutically acceptable form thereof, corresponds to the formulae (I-e) or (I-e′):

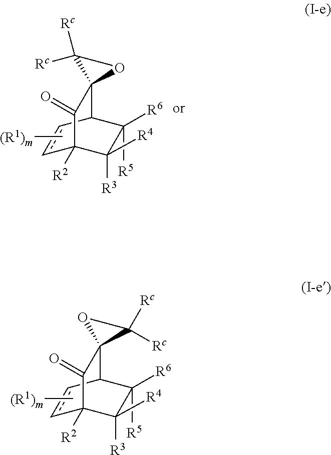

wherein

R¹, R², R³, R⁴, R⁵, R⁶, R^{C}, m, and n are as defined above and herein.

In certain embodiments, the inventive compound of formula (I), or a pharmaceutically acceptable form thereof, corresponds to the formulae (I-f) or (I-f′):

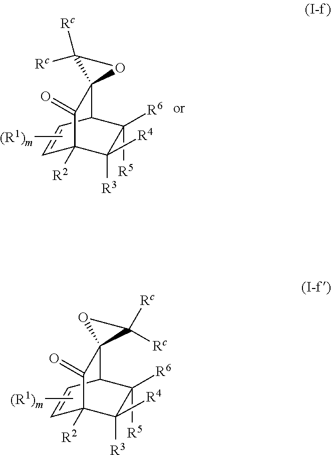

wherein

R¹, R², R³, R⁴, R⁵, R⁶, and R^{C} are as defined above and herein; and

m is 0, 1, or 2.

In certain embodiments, the inventive compound of formula (I), or a pharmaceutically acceptable form thereof, corresponds to the formula (I-g) or (I-g′):

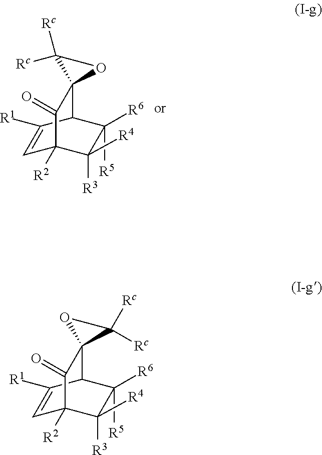

wherein

R¹, R², R³, R⁴, R⁵, R⁶, and R^{C} are as defined above and herein.

In certain embodiments, the inventive compound of formula (I), or a pharmaceutically acceptable form thereof, corresponds to the formula (I-h) or (I-h′):

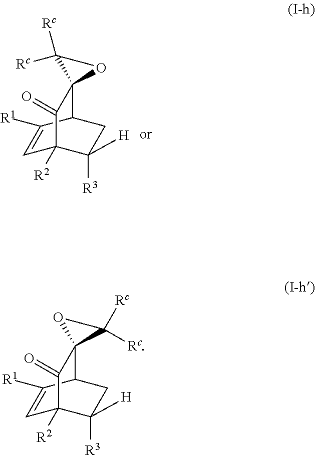

wherein

R¹, R², R³, and R^{C} are as defined above and herein.

In certain embodiments, the inventive compound of formula (I), or a pharmaceutically acceptable form thereof, corresponds to the formula (I-i) or (I-i′):

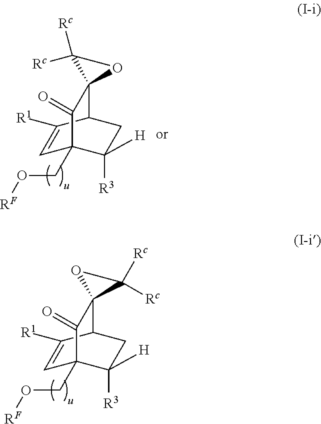

wherein

R¹, R³, and R^{C} are as defined above and herein;

R^{F} is hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or a suitable hydroxyl protecting group; and

u is 1, 2, 3, 4, 5, or 6.

In certain embodiments, R^{F} is hydrogen; cyclic or acyclic, substituted or unsubstituted, branched or unbranched aliphatic; cyclic or acyclic, substituted or unsubstituted, branched or unbranched heteroaliphatic; or a suitable hydroxyl protecting group.

In certain embodiments, R^{F} is hydrogen; cyclic or acyclic, substituted or unsubstituted, branched or unbranched C₁₋₆ aliphatic; cyclic or acyclic, substituted or unsubstituted, branched or unbranched C₁₋₆ heteroaliphatic; or a suitable hydroxyl protecting group. In certain embodiments, R^{F} is hydrogen or cyclic or acyclic, substituted or unsubstituted, branched or unbranched C₁₋₆ aliphatic. In certain embodiments, R^{F} is an acyclic, substituted or unsubstituted, branched or unbranched C₁₋₆ aliphatic. In certain embodiments, R^{F} is an acyclic, unsubstituted, branched or unbranched C₁₋₆ aliphatic. In certain embodiments, R^{F} is an acyclic, unsubstituted, unbranched C₁₋₆ aliphatic. In certain embodiments, R^{F} is —CH₃. In certain embodiments, R^{F} is hydrogen.

In certain embodiments, u is 1, 2, or 3. In certain embodiments, u is 1.

In certain embodiments, the inventive compound of formula (I) is any one of the following compounds:

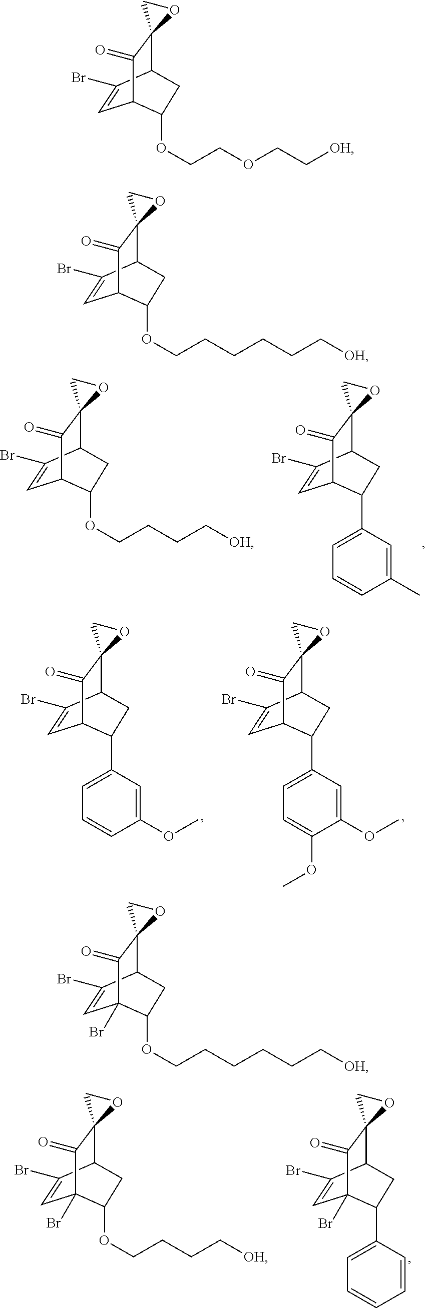

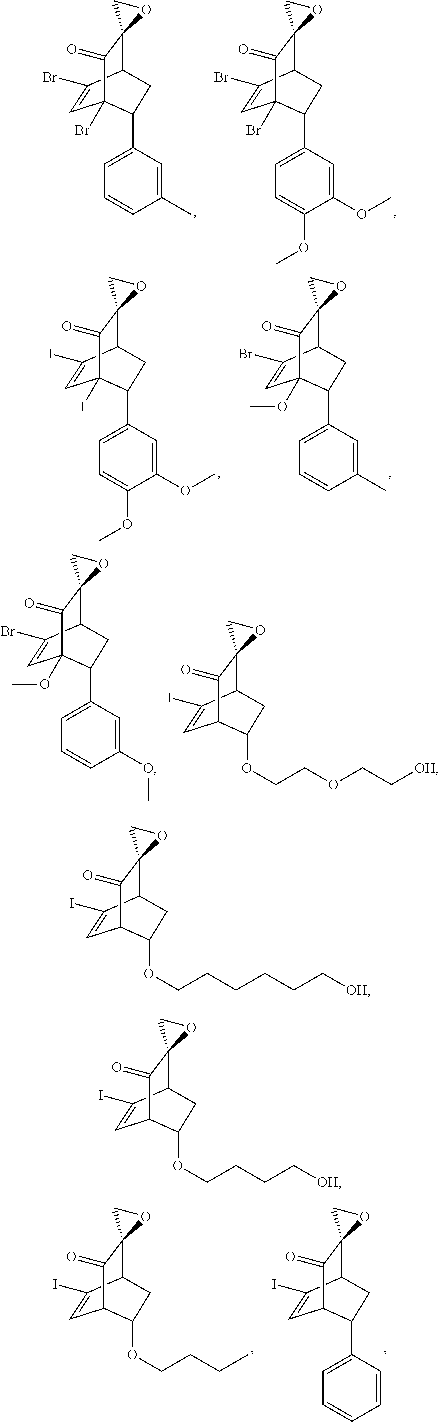

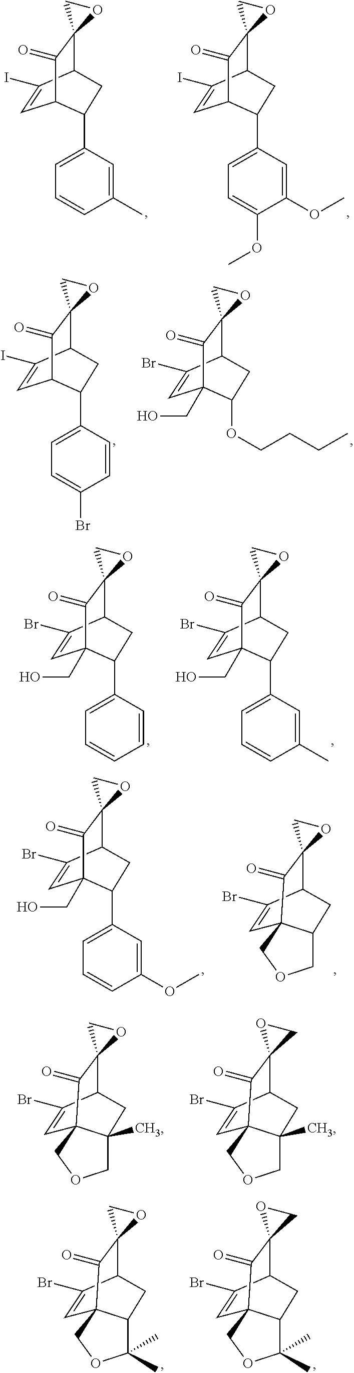

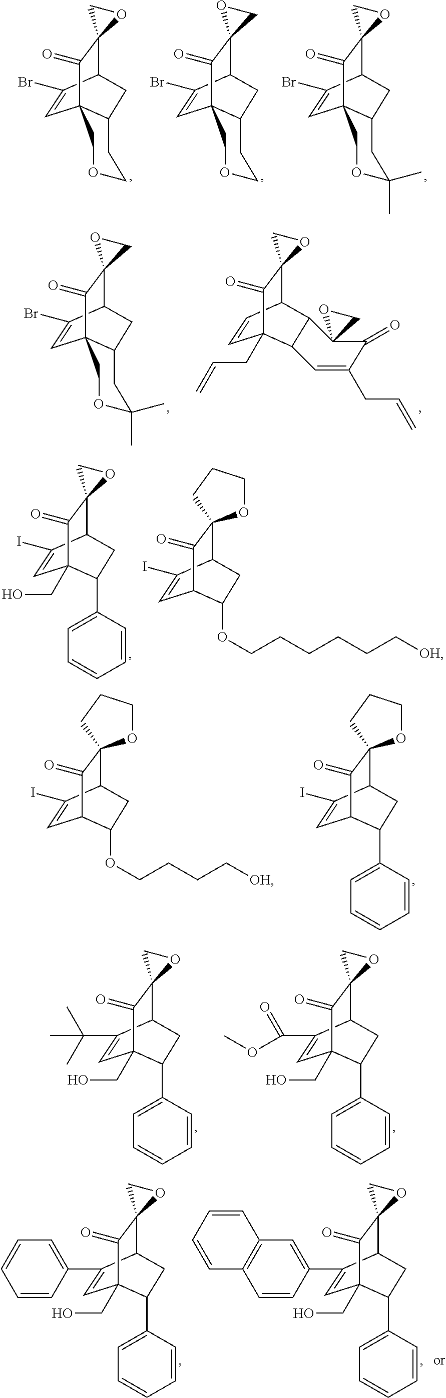

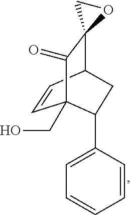

or a pharmaceutically acceptable form thereof.

In certain embodiments, the inventive compound of formula (I) is any one of the following compounds:

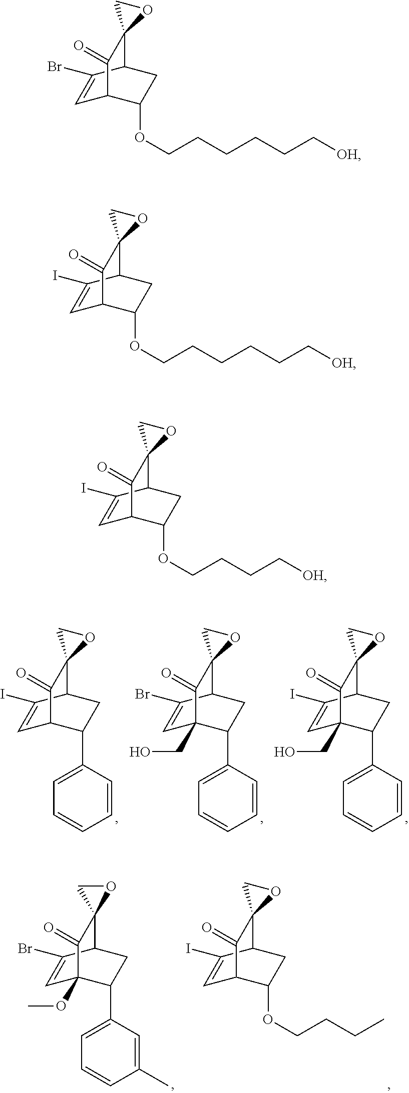

or a pharmaceutically acceptable form thereof.

In certain embodiments, the inventive compound of formula (I) is any one of the following compounds:

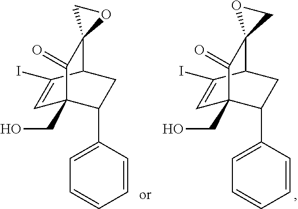

or a pharmaceutically acceptable form thereof.

In certain embodiments, 5-bromo-endo-7-phenyl-exo-3-spiroepoxybicyclo[2.2.2]oct-5-en-2-one as an inventive compound of formula (I) is specifically excluded (see the four stereoisomers of 5-bromo-endo-7-phenyl-exo-3-spiroepoxybicyclo[2.2.2]oct-5-en-2-one depicted below).

In certain embodiments, the alpha, syn isomer of 5-bromo-endo-7-phenyl-exo-3-spiroepoxybicyclo[2.2.2]oct-5-en-2-one is excluded.

In certain embodiments, the beta, syn isomer of 5-bromo-endo-7-phenyl-exo-3-spiroepoxybicyclo[2.2.2]oct-5-en-2-one is excluded.

In certain embodiments, the alpha, anti isomer of 5-bromo-endo-7-phenyl-exo-3-spiroepoxybicyclo[2.2.2]oct-5-en-2-one is excluded.

In certain embodiments, the beta, anti isomer of 5-bromo-endo-7-phenyl-exo-3-spiroepoxybicyclo[2.2.2]oct-5-en-2-one is excluded.

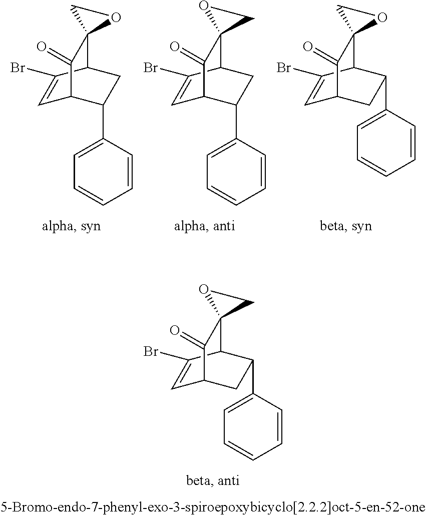

In certain embodiments, 5-bromo-exo-7-phenyl-exo-3-spiroepoxybicyclo[2.2.2]oct-5-en-2-one as an inventive compound of formula (I) is specifically excluded (see the four stereoisomers of 5-bromo-exo-7-phenyl-exo-3-spiroepoxybicyclo[2.2.2]oct-5-en-2-one depicted below).

In certain embodiments, the alpha, syn isomer of 5-bromo-exo-7-phenyl-exo-3-spiroepoxybicyclo[2.2.2]oct-5-en-2-one is excluded.

In certain embodiments, the beta, syn isomer of 5-bromo-exo-7-phenyl-exo-3-spiroepoxybicyclo[2.2.2]oct-5-en-2-one is excluded.

In certain embodiments, the alpha, anti isomer of 5-bromo-exo-7-phenyl-exo-3-spiroepoxybicyclo[2.2.2]oct-5-en-2-one is excluded.

In certain embodiments, the beta, anti isomer of 5-bromo-exo-7-phenyl-exo-3-spiroepoxybicyclo[2.2.2]oct-5-en-2-one is excluded.

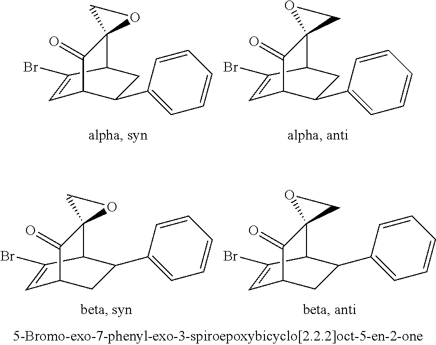

The present invention also provides a compound, or a pharmaceutically acceptable form thereof, having the formula:

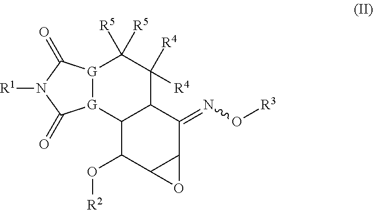

wherein

each instance of G is, independently, —N— or —(CH)—;

R¹ is hydrogen; hydroxyl; halogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable amino protecting group;

R² is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable hydroxyl protecting group;

R³ is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; substituted or unsubstituted acyl; or a suitable hydroxyl protecting group; and

each instance of R⁴ is, independently, hydrogen; halogen; substituted or unsubstituted hydroxyl; substituted or unsubstituted amino; substituted or unsubstituted thiol; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or substituted or unsubstituted acyl; or two R⁴ groups taken together form an oxo (═O), an imino (═NH), or a thiooxo (═S) group; and

each instance of R⁵ is, independently, hydrogen; hydrogen; halogen; substituted or unsubstituted hydroxyl; substituted or unsubstituted amino; substituted or unsubstituted thiol; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or substituted or unsubstituted acyl; or two R⁵ groups taken together form an oxo (═O), an imino (═NH), or a thiooxo (═S) group.

In certain embodiments, each instance of G is —N—. In certain embodiments, each instance of G is —(CH)—.

In certain embodiments, R¹ is cyclic or acyclic, substituted or unsubstituted, branched or unbranched C₁₋₂₀ aliphatic; cyclic or acyclic, substituted or unsubstituted, branched or unbranched C₁₋₂₀ heteroaliphatic; cyclic or acyclic, substituted or unsubstituted C₁₋₂₀ aryl; cyclic or acyclic, substituted or unsubstituted C₁₋₂₀ heteroaryl; substituted or unsubstituted C₁₋₂₀ acyl; or a suitable amino protecting group. In certain embodiments, R¹ is cyclic or acyclic, substituted or unsubstituted, branched or unbranched C₁₋₁₀ aliphatic; cyclic or acyclic, substituted or unsubstituted, branched or unbranched C₁₋₁₀ heteroaliphatic; cyclic or acyclic, substituted or unsubstituted C₁₋₁₀ aryl; cyclic or acyclic, substituted or unsubstituted C₁₋₁₀ heteroaryl; substituted or unsubstituted C₁₋₁₀ acyl; or a suitable amino protecting group. In certain embodiments, R¹ is cyclic or acyclic, substituted or unsubstituted, branched or unbranched C₁₋₈ aliphatic; cyclic or acyclic, substituted or unsubstituted, branched or unbranched C₁₋₈ heteroaliphatic; cyclic or acyclic, substituted or unsubstituted C₁₋₈ aryl; cyclic or acyclic, substituted or unsubstituted C₁₋₈ heteroaryl; substituted or unsubstituted C₁₋₈ acyl; or a suitable amino protecting group. In certain embodiments, R¹ is cyclic or acyclic, substituted or unsubstituted, branched or unbranched C₁₋₆ aliphatic; cyclic or acyclic, substituted or unsubstituted, branched or unbranched C₁₋₆heteroaliphatic; cyclic or acyclic, substituted or unsubstituted C₁₋₆ aryl; cyclic or acyclic, substituted or unsubstituted C₁₋₆heteroaryl; substituted or unsubstituted C₁₋₆ acyl; or a suitable amino protecting group.

In certain embodiments, R¹ is hydrogen. In certain embodiments, R¹ is cyclic or acyclic, substituted or unsubstituted, branched or unbranched aliphatic. In certain embodiments, R¹ is cyclic or acyclic, substituted or unsubstituted, branched or unbranched heteroaliphatic. In certain embodiments, R¹ is substituted or unsubstituted aryl. In certain embodiments, R¹ is substituted or unsubstituted heteroaryl. In certain embodiments, R¹ is substituted or unsubstituted acyl. In certain embodiments, R¹ is a suitable amino protecting group. In certain embodiments, R¹ is substituted or unsubstituted arylalkyl.

In certain embodiments, R² is hydrogen; or cyclic or acyclic, substituted or unsubstituted, branched or unbranched aliphatic. In certain embodiments, R² is hydrogen.

In certain embodiments, R³ is cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or a suitable hydroxyl protecting group.

In certain embodiments, each instance of R⁴ and R⁵ is, independently, hydrogen; cyclic or acyclic, or substituted or unsubstituted, branched or unbranched aliphatic. In certain embodiments, R⁴ is hydrogen. In certain embodiments, R⁵ is hydrogen. In certain embodiments, each instance of R⁴ and R⁵ is hydrogen.

In certain embodiments, the inventive compound of formula (II) corresponds to the formulae (II-a) or (II-b):

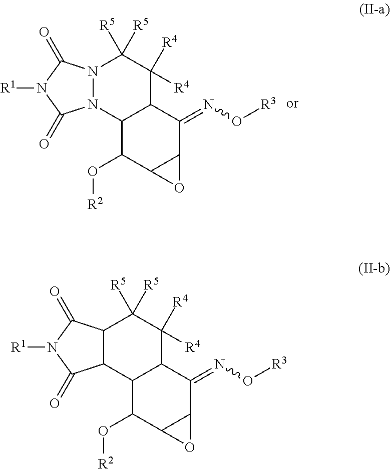

wherein R¹, R², R³, R⁴, and R⁵ are as defined above and herein.

In certain embodiments, the inventive compound of formula (II) corresponds to the formulae (II-c) or (II-d):

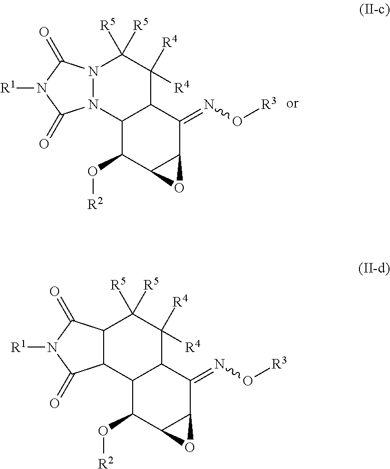

wherein R¹, R², R³, R⁴, and R⁵ are as defined above and herein.

In certain embodiments, the inventive compound of formula (II) corresponds to the formula (II-e) or (II-f):

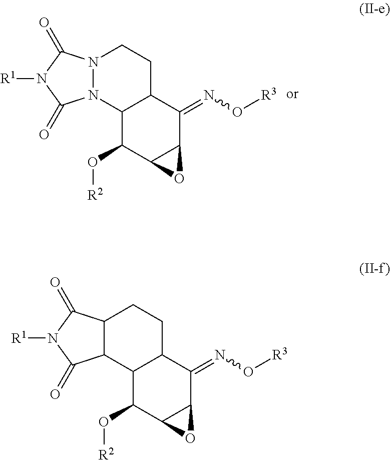

wherein R¹, R², and R³ are as defined above and herein.

In certain embodiments, the inventive compound of formula (II) corresponds to the formula (II-g) or (II-h):

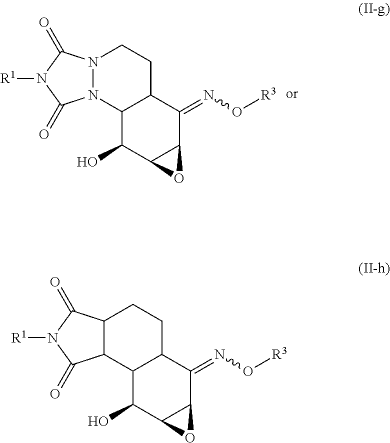

wherein R¹, R², and R³ are as defined above and herein.

In certain embodiments, an inventive compound of formula (II) corresponds to any one of the following compounds:

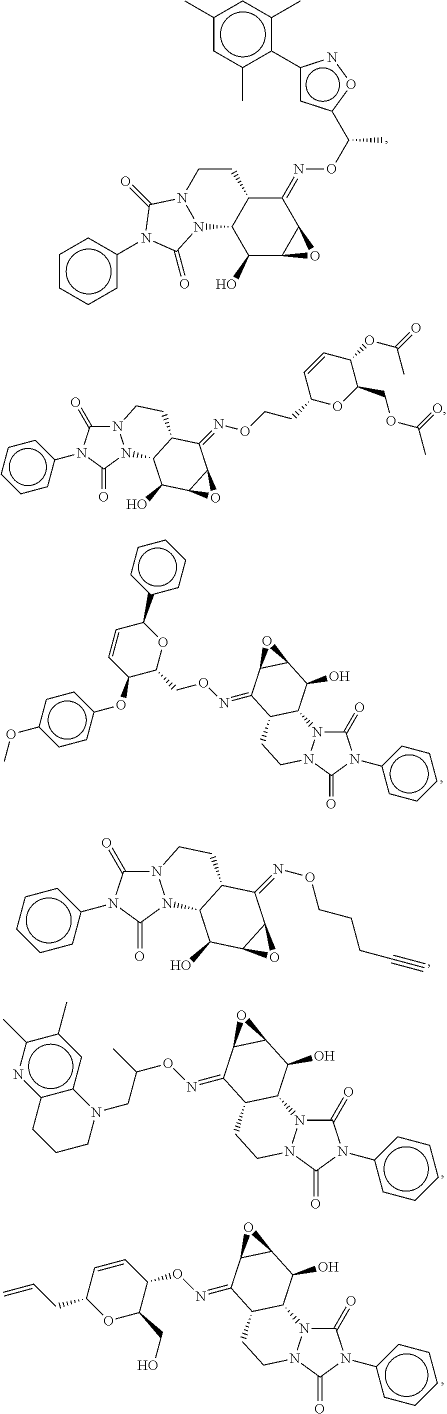

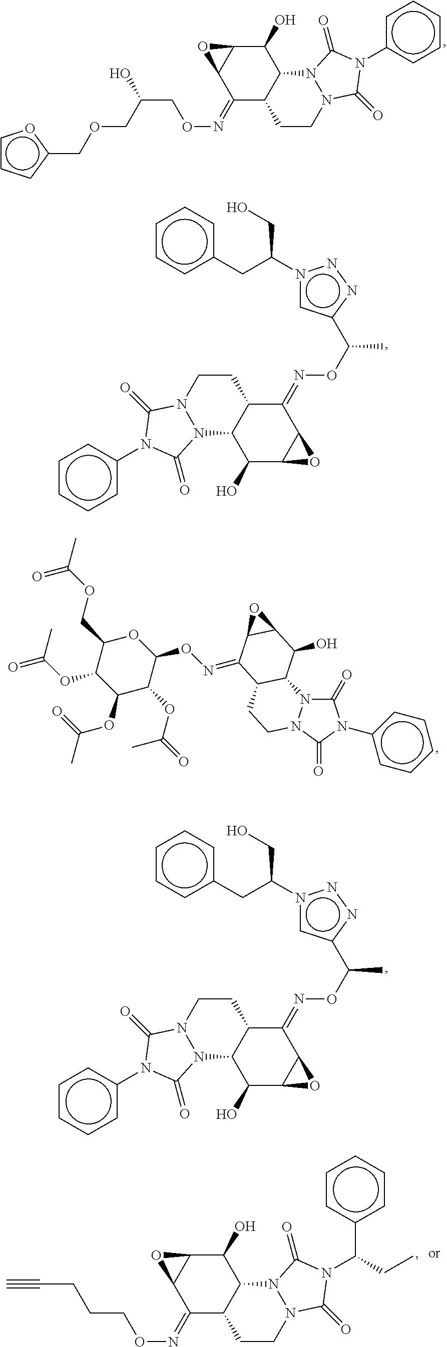

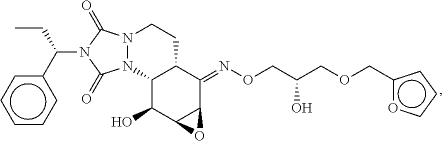

or a pharmaceutically acceptable form thereof.

### Pharmaceutical Compositions

The present invention also provides pharmaceutical compositions comprising one or more inventive compounds corresponding to any of the above formulae as described herein (e.g., (I), (II), and subsets thereof), or a pharmaceutically acceptable form thereof, and a pharmaceutically acceptable excipient.

In accordance with some embodiments, a method of administering a pharmaceutical composition comprising inventive compositions to a subject in need thereof is provided. In some embodiments, inventive compositions are administered to humans. For the purposes of the present invention, the phrase “active ingredient” generally refers to an inventive compound, as described above and herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

The formulations of the pharmaceutical compositions described herein may be prepared by any method known or hereafter developed in the art of pharmacology. In general, such preparatory methods include the step of bringing the active ingredient into association with a carrier and/or one or more other accessory ingredients, and then, if necessary and/or desirable, shaping and/or packaging the product into a desired single- or multi-dose unit.

A pharmaceutical composition of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Exemplary pharmaceutically acceptable excipients include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form. Remington's The Science and Practice of Pharmacy, 21^{st} Edition, A. R. Gennaro, (Lippincott, Williams & Wilkins, Baltimore, Md., 2006) discloses various carriers used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfate, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a conjugate of this invention may include ointments, pastes, creams, lotions, gels, powders, solutions, sprays, inhalants and/or patches. Generally, the active component is admixed under sterile conditions with a pharmaceutically acceptable carrier and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65 .degree. F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may comprise one or more of the additional ingredients described herein. A pharmaceutical composition of the invention may be prepared, packaged, and/or sold in a formulation suitable for buccal administration. Such formulations may, for example, be in the form of tablets and/or lozenges made using conventional methods, and may, for example, 0.1 to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition of the invention may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

### Administration

In some embodiments, a therapeutically effective amount of an inventive pharmaceutical composition is delivered to a patient and/or organism prior to, simultaneously with, and/or after diagnosis with a disease, disorder, and/or condition. In some embodiments, a therapeutic amount of an inventive composition is delivered to a patient and/or organism prior to, simultaneously with, and/or after onset of symptoms of a disease, disorder, and/or condition. In some embodiments, the amount of inventive conjugate is sufficient to treat, alleviate, ameliorate, relieve, delay onset of, inhibit progression of, reduce severity of, and/or reduce incidence of one or more symptoms or features of the disease, disorder, and/or condition.

The compositions, according to the method of the present invention, may be administered using any amount and any route of administration effective for treatment. The exact amount required will vary from subject to subject, depending on the species, age, and general condition of the subject, the severity of the infection, the particular composition, its mode of administration, its mode of activity, and the like. The compositions of the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The pharmaceutical compositions of the present invention may be administered by any route. In some embodiments, the pharmaceutical compositions of the present invention are administered variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In general the most appropriate route of administration will depend upon a variety of factors including the nature of the agent (e.g., its stability in the environment of the gastrointestinal tract), the condition of the subject (e.g., whether the subject is able to tolerate oral administration), etc. At present the oral and/or nasal spray and/or aerosol route is most commonly used to deliver therapeutic agents directly to the lungs and/or respiratory system. However, the invention encompasses the delivery of the inventive pharmaceutical composition by any appropriate route taking into consideration likely advances in the sciences of drug delivery.

The exact amount of a compound provided in a pharmaceutical composition of the present invention required to achieve a therapeutically effective amount will vary from subject to subject, depending on species, age, and general condition of a subject, severity of the side effects or disorder, identity of the particular compound(s), mode of administration, and the like. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In certain embodiments of the present invention, a therapeutically effective amount of an inventive compound for administration one or more times a day to a 70 kg adult human may comprise about 0.0001 mg to about 1000 mg of an inventive compound per unit dosage form. It will be appreciated that dose ranges as described herein provide guidance for the administration of inventive pharmaceutical compositions to an adult. The amount to be administered to, for example, a child or an adolescent can be determined by a medical practitioner or person skilled in the art and can be lower or the same as that administered to an adult.

In certain embodiments, the inventive pharmaceutical composition comprises about 0.0001 mg to about 1000 mg of an inventive compound per unit dosage form. In certain embodiments, the composition comprises from about 0.0001 mg to about 1000 mg, from about 0.001 mg to about 1000 mg, from about 0.01 mg to about 1000 mg, from about 0.01 mg to about 1000 mg, from about 0.1 mg to about 1000 mg, from about 1 mg to about 1000 mg, from about 2 mg to about 1000 mg, from about 4 mg to about 1000 mg, from about 6 mg to about 1000 mg, from about 8 mg to about 1000 mg, from about 10 mg to about 1000 mg, from about 20 mg to about 1000 mg, from about 30 mg to about 1000 mg, from about 40 mg to about 1000 mg, from about 60 mg to about 1000 mg, from about 80 mg to about 1000 mg, from about 100 mg to about 1000 mg, from about 200 mg to about 1000 mg, from about 300 mg to about 1000 mg, from about 400 mg to about 1000 mg, from about 500 mg to about 1000 mg, from about 600 mg to about 1000 mg, from about 700 mg to about 1000 mg, from about 800 mg to about 1000 mg, from about 900 mg to about 1000 mg; from about 0.0001 mg to about 1000 mg, from about 0.0001 mg to about 900 mg, from about 0.0001 mg to about 800 mg, from about 0.0001 mg to about 700 mg, from about 0.0001 mg to about 600 mg, from about 0.0001 mg to about 500 mg, from about 0.0001 mg to about 400 mg, from about 0.0001 mg to about 300 mg, from about 0.0001 mg to about 200 mg, from about 0.0001 mg to about 100 mg, from about 0.0001 mg to about 90 mg, from about 0.0001 mg to about 80 mg, from about 0.0001 mg to about 70 mg, from about 0.0001 mg to about 60 mg, from about 0.0001 mg to about 50 mg, from about 0.0001 mg to about 40 mg, from about 0.0001 mg to about 30 mg, from about 0.0001 mg to about 20 mg, from about 0.0001 mg to about 10 mg, from about 0.0001 mg to about 8 mg, from about 0.0001 mg to about 6 mg, from about 0.0001 mg to about 4 mg, from about 0.0001 mg to about 2 mg; from about 0.0001 mg to about 1 mg; from about 0.0001 mg to about 0.1 mg; from about 0.0001 mg to about 0.01 mg; from about 0.0001 mg to about 0.001 mg of an inventive compound per unit dosage form. In certain embodiments, the composition comprises at least about 0.0001 mg, at least about 0.001 mg, at least about 0.01 mg, at least about 0.1 mg, at least about 1 mg, at least about 2 mg, at least about 4 mg, at least about 6 mg, at least about 8 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 560 mg, at least about 580 mg, at least about 600 mg, at least about 620 mg, at least about 640 mg, at least about 660 mg, at least about 680 mg, at least about 700 mg, at least about 720 mg, at least about 740 mg, at least about 760 mg, at least about 780 mg, at least about 800 mg, at least about 820 mg, at least about 840 mg, at least about 860 mg, at least about 880 mg, at least about 900 mg, at least about 920 mg, at least about 940 mg, at least about 960 mg, at least about 980 mg, or at least about 1000 mg of an inventive compound per unit dosage form.

It will be also appreciated that an inventive pharmaceutical composition, as described above and herein, can be employed in combination therapies, that is, an inventive pharmaceutical composition can be administered concurrently with, prior to, or subsequent to, one or more other desired therapeutics or medical procedures. Particular combination therapies (therapeutics or procedures) to employ in a combination regimen will take into account compatibility of the desired therapeutics and/or procedures and the desired therapeutic effect to be achieved. It will also be appreciated that therapies employed may achieve a desired effect for the same disorder (for example, an inventive pharmaceutical composition may be administered concurrently with another therapeutically active agent used to treat the same disorder), or they may achieve different effects (e.g., control of any adverse effects). As used herein, additional therapeutic compounds which are normally administered to treat or prevent a particular disease, or condition, are known as “appropriate for the disease, or condition, being treated.”

Pharmaceutical compositions of the present invention may be administered either alone or in combination with one or more other biologically active agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of the inventive pharmaceutical compositions in combination with agents that may improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an inventive polypeptide may be administered concurrently with another biologically active agent used to treat the same disorder), and/or they may achieve different effects (e.g., control of any adverse effects). In some embodiments, polypeptides of the invention are administered with a second biologically active agent that is approved by the U.S. Food and Drug Administration.

In will further be appreciated that the additional biologically active agents utilized in this combination may be administered together in a single composition or administered separately in different compositions.

In general, it is expected that biologically active agents utilized in combination be utilized at levels that do not exceed the levels at which they are utilized individually. In some embodiments, the levels utilized in combination will be lower than those utilized individually.

### Kits

Still further encompassed by the invention are kits comprising one or more inventive compounds (or pharmaceutically acceptable forms thereof), and/or an inventive pharmaceutical composition. Kits are typically provided in a suitable container (e.g., for example, a foil, plastic, or cardboard package). In certain embodiments, an inventive kit may include one or more pharmaceutical excipients, pharmaceutical additives, therapeutically active agents, and the like, as described herein. In certain embodiments, an inventive kit may include means for proper administration, such as, for example, graduated cups, syringes, needles, cleaning aids, and the like. In certain embodiments, an inventive kit may include instructions for proper administration and/or preparation for proper administration.

### Methods of Treatment

The present invention also provides methods of treating a viral infection comprising administering to a subject diagnosed with or being susceptible to a viral infection a therapeutically effective amount of an inventive compound, or pharmaceutical composition thereof, wherein the compound corresponds to any of the above formulae as described herein (e.g., (I), (II), and subsets thereof) or a pharmaceutical form thereof.

Exemplary viral infections include, but are not limited to, DNA viruses, RNA viruses, varicella (chickenpox) virus, hantavirus, hepatitis (e.g., hepatitis A, hepatitis B, hepatitis C, hepatitis D, and hepatitis E), herpes (e.g., herpes simplex virus type 1, herpes simplex virus type 2, varicella-zoster virus, Epstein-Barr virus, Cytomegalovirus, herpesvirus 6 and herpesvirus 7, herpesvirus 8), measles, monkeypox virus, rabies, respiratory syncytial virus, West Nile virus, human immunodeficiency virus (HIV), adenovirus, Coxsackie virus, human parainfluenza virus (e.g., Influenza A and B), warts, bronchitis, and encephalitis virus viral infections.

In certain embodiments, the viral infection is a hepatitis viral infection, or a viral infection which may result in a heptatitis viral infection. In certain embodiments, the viral infection is a hepatitis A infection. In certain embodiments, the viral infection is a hepatitis B infection. In certain embodiments, the viral infection is a hepatitis C infection. In certain embodiments, the viral infection is a hepatitis D infection. In certain embodiments, the viral infection is a hepatitis E infection. In certain embodiments, the viral infection is an infection caused by one or more hepatits viruses (e.g., hepatitis A, hepatitis B, hepatitis C, hepatitis D, and/or hepatitis E). In certain embodiments, a viral infection which may result in a heptatitis viral infection is selected from Epstein Barr viral infection, varicella, and cytomegalovirus (CMV) infection.

### Screening and Assays

The present invention also provides a high-throughput method of identifying compounds which modulate HCV replication activity, comprising the steps of: (i) providing a multiwell plate comprising at least about 90 wells per plate; (ii) adding at least one HCV replicon cell to said wells; (iii) providing at least one test compound; (iv) contacting the test compound to the HCV replicon cell under suitable conditions to illicit a change in HCV RNA replication activity; and (v) detecting a change in luciferase activity, wherein said change in luciferase activity is directly proportional to said change in HCV replication activity.

In certain embodiments, the at least one test compound of step (iii) is a member of a DOS library of compounds.

In certain embodiments, the HCV replicon cell is Huh7/Rep-Feo HCV replicon cell.

In certain embodiments, multiwell plate of step (i) comprises at least about 100 wells per plate. In certain embodiments, multiwell plate of step (i) comprises at least about 150 wells per plate. In certain embodiments, multiwell plate of step (i) comprises at least about 200 wells per plate. In certain embodiments, the multiwell plate of step (i) comprises at least about 250 wells per plate. In certain embodiments, multiwell plate of step (i) comprises at least about 300 wells per plate. In certain embodiments, multiwell plate of step (i) comprises at least about 350 wells per plate. In certain embodiments, multiwell plate of step (i) comprises at least about 400 wells per plate. In certain embodiments, multiwell plate of step (i) comprises at least about 450 wells per plate. In certain embodiments, the multiwell plate of step (i) comprises at least about 500 wells per plate. In certain embodiments, multiwell plate of step (i) comprises at least about 550 wells per plate. In certain embodiments, multiwell plate of step (i) comprises at least about 600 wells per plate. In certain embodiments, multiwell plate of step (i) comprises at least about 650 wells per plate. In certain embodiments, multiwell plate of step (i) comprises at least about 700 wells per plate. In certain embodiments, multiwell plate of step (i) comprises at least about 750 wells per plate. In certain embodiments, multiwell plate of step (i) comprises at least about 800 wells per plate. In certain embodiments, multiwell plate of step (i) comprises at least about 850 wells per plate. In certain embodiments, multiwell plate of step (i) comprises at least about 900 wells per plate. In certain embodiments, multiwell plate of step (i) comprises at least about 950 wells per plate. In certain embodiments, multiwell plate of step (i) comprises at least about 1000 wells per plate.

In certain embodiments, the above method further comprises (vi) providing a second multiwell plate comprising at least about one well per plate; (vii) adding at least adding at least one HCV infected cell; (viii) providing at least one test compound; (ix) contacting the compound to the cell of step (vii); and (x) assessing the cytotoxicity of the test compound to the HCV infected cell of step (vii).

In certain embodiments, the second multiwell plate of step (vi) comprises at least about 25 wells per plate. In certain embodiments, the second multiwell plate of step (vi) comprises at least about 50 wells per plate. In certain embodiments, the second multiwell plate of step (vi) comprises at least about 75 wells per plate. In certain embodiments, the second multiwell plate of step (vi) comprises at least about 100 wells per plate. In certain embodiments, the second multiwell plate of step (vi) comprises at least about 150 wells per plate. In certain embodiments, the second multiwell plate of step (vi) comprises at least about 200 wells per plate. In certain embodiments, the second multiwell plate of step (vi) comprises at least about 250 wells per plate. In certain embodiments, the second multiwell plate of step (vi) comprises at least about 300 wells per plate. In certain embodiments, the second multiwell plate of step (vi) comprises at least about 350 wells per plate. In certain embodiments, the second multiwell plate of step (vi) comprises at least about 400 wells per plate. In certain embodiments, the second multiwell plate of step (vi) comprises at least about 450 wells per plate. In certain embodiments, the second multiwell plate of step (vi) comprises at least about 500 wells per plate. In certain embodiments, the second multiwell plate of step (vi) comprises at least about 550 wells per plate. In certain embodiments, the second multiwell plate of step (vi) comprises at least about 600 wells per plate. In certain embodiments, the second multiwell plate of step (vi) comprises at least about 650 wells per plate. In certain embodiments, the second multiwell plate of step (vi) comprises at least about 700 wells per plate. In certain embodiments, the second multiwell plate of step (vi) comprises at least about 750 wells per plate. In certain embodiments, the second multiwell plate of step (vi) comprises at least about 800 wells per plate. In certain embodiments, the second multiwell plate of step (vi) comprises at least about 850 wells per plate. In certain embodiments, the second multiwell plate of step (vi) comprises at least about 900 wells per plate. In certain embodiments, the second multiwell plate of step (vi) comprises at least about 950 wells per plate. In certain embodiments, the second multiwell plate of step (vi) comprises at least about 1000 wells per plate.

As used herein “HCV infected cell” refers to a cell which has been infected with HCV or one or more HCV viral particles, or the cell provides intracellular HCV replication by use of an HCV replicon system (e.g., a Huh7/Rep-Feo HCV replicon cell). In certain embodiments, the HCV viral particles are obtained from an HCV genome that replicates and produces RNA virus particles infectious in cell culture (HCVcc). HCV is an enveloped, positive-sense RNA virus of the family Flaviviridae. Naturally occurring variants of HCV are classified into six major genotypes. The 9.6-kb genome encodes one large polyprotein that is processed by viral and cellular proteinases to produce the virion structural proteins (core and glycoproteins E1 and E2) as well as nonstructural (NS) proteins (p7 through NS5B). Subgenomic RNA replicons have been adapted for efficient RNA replication in the human hepatoma line Huh-7 and other cultured cells (see for example, Lindenbach et al., Science (2005) 309:623-626; Blight et al., Science (2000) 290:1972; Date et al., J. Biol. Chem. (2004) 279:22371; Kato et al., Gastroenterology (2003) 125:1808; Lohmann et al., Science (1999) 285:110; Tanabe et al., Journal of Infectious Diseases (2004) 189:1129-1139; and Naka et al., Biochem. Biophys. Res. Commun. (2005) 329:1350-1359; the entire contents of each of which are hereby incorporated herein by reference). Thus, the present invention contemplates that many different type of cell cultures, and HCV genomes used to infect these cell cultures, may be used to successfully set-up and perform the secondary validation assay of method steps (vi) to (x). Suitable HCV genomes that replicate and produce RNA virus particles infectious in cell culture (HCVcc) include, but are not limited to, JFH-1, Com1/JFH-1, SGR-JFH1, FL-J6/JFH, and FL-H77/JFH.

In certain embodiments, the cell of step (vii) is different from the HCV replicon cell of step (ii). In certain embodiments, the cell of step (vii) is an OR6 cell or an a Huh-7.5 cell. In certain embodiments, the cell of step (vii) is an OR6 cell stably harboring ORN/C-5B/KE.

In order that the invention described herein may be more fully understood, the following examples are set forth. It should be understood that these examples are for illustrative purposes only and are not to be construed as limiting this invention in any manner.

### EXAMPLES

### Example 1

### A Cell-Based, High Throughput Screen for Small Molecule Regulators of Hepatitis C Virus Replication

A cell-based HTS assay using 384-well plates has been developed with an HCV replicon bearing a beta-lactamase reporter gene (Murray E M, Grobler J A, Markel E J, Pagnoni M F, Paonessa G, Simon A J, Flores O A. Persistence replication of hepatitis C virus replicons expressing the beta-lactamase reporter in subpopulations of highly permissive Huh7 cells. J Virol 2003; 77: 2928-2935). However, this replicon model has several disadvantages. There is a relatively low signal to background. Moreover, additional processing is required to suppress the high background signal. Finally, because the cell line was transiently transfected, it requires extensive preparation prior to screening. Renilla luciferase has also been used as a reporter gene, but is not an ideal choice because of its very short signal half-life. Moreover, aspiration and lysis processing steps must be carried out prior to signal detection.

On the other hand, replicon cell models bearing a SEAP reporter do not require aspiration and lysis steps. These systems, however, either require another viral protein, such as tat, to express the reporter protein or require the action of NS3/4A-specific protease activity.

Development of the Huh7/Rep-Feo replicon assay. The subgenomic Huh7/Rep-Feo HCV replicon cell line appears to be particularly well suited to automated HTS methods was selected for further assay development. This replicon was derived from a chimpanzee infectious clone (strain HCV—N, genotype 1b). In this replicon, the structural genes have been replaced by a reporter gene. The chimeric reporter gene Feo encodes the firefly luciferase protein fused in-frame with neomycin phosphotransferase. This Huh7/Rep-Feo cell supports high levels of autonomous HCV RNA replication because it was derived from the HCV—N strain, which carries an adaptive mutation in NSSA that confers highlevel replication in tissue culture. Furthermore, the level of luciferase correlates well with levels of HCV RNA production, so that luciferase can be used as a reliable surrogate marker for HCV replication. The use of luciferase as a reporter permits quantitative and highthroughput detection of HCV replication levels. The specific use of firefly luciferase makes this replicon especially well-suited for automation, because the luciferase reagent can be added directly to the cell culture system prior to signal detection without the need for cell lysis, washing, or aspiration.

Replicons bearing firefly luciferase reporter genes appear to be better suited for use in HTS assays. Many small molecules are cytotoxic, and hepatocyte replicon cell lines are highly sensitive to cytotoxic or cytostatic agents. Cytotoxic effects can be mistaken for antiviral activity by decreasing luciferase signal merely by decreasing cell viability and not by decreasing HCV RNA replication, leading to false-positive results. Therefore, both the reporter gene assay and a cell viability assay should be performed in parallel in the primary HTS. This counter screen should be executed in order to minimize confounding from increased or decreased luciferase signal due to increased or decreased cell viability, respectively.

In the secondary screen, primary hits were validated using a full-length OR6 replicon, thereby ensuring validation in a more authentic viral polyprotein context. This full-length replicon also possesses a cell cultureadaptive mutation and a reporter gene distinct from those found in the subgenomic Huh7/Rep-Feo replicon, thereby minimizing confounding from those factors. Secondary validation screens were conducted to generate adequate dose-response curves for the hit compounds. Cell viability assays were performed in the secondary screens to minimize confounding from cytotoxicity.

HCV Replicon System-Primary Screening. The Huh7/pRep-Feo replicon cell line that was used for these studies has been described previously (Everson, et al., Liver Transpl. (2002) 8:S19-S275; Ye, et al., Proc. Natl. Acad. Sci. USA (2003) 100:15865-15870; the entirety of which is hereby incorported herein by reference). The HCV Replicon assay has also been described (Kim et al., Gastroenterology (2007) 132:311-320, the entirety of which is hereby incorporated herein by reference). Briefly, this replicon harbors a subgenomic HCV genotype 1b sequence, fused to firefly luciferase, stably replicating under neomycin selection. Cells were propagated in Dulbecco's Modified Eagle's medium (DMEM) containing 10% fetal bovine serum (FBS) supplemented with 1% penicillin-streptomycin, and 500 μg of Geneticin (Invitrogen Corp., Carlsbad, Calif.)/mL. Cells were cultured in a 37° C., 5% CO2-humidified incubator for all experiments. To decrease day-today variability in the assay, a large homogenous population of subconfluent cells was passaged so that a similar lot of cells could be used throughout the HTS assay.

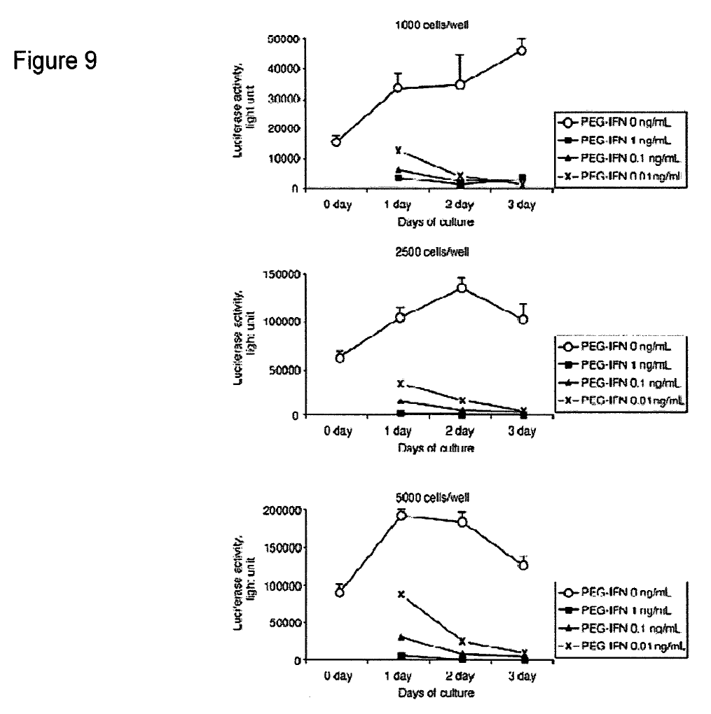

Optimization of Huh-7/Rep-Feo Cells for the 384-Well Plate Format. The Huh7/Rep-Feo cells were first optimized for the 96-well plate format (FIG. 9). Peginterferon-alfa-2b (PEG-Intron; Schering Corp., Kenilworth, N.J.) was used as a positive control for inhibition. Cells were seeded at densities of 5000 and 10,000 cells/well in 100_L of medium in 96-well plates. The cells were allowed to attach overnight (approximately 24 hours) before addition of PEG-IFN at various concentrations (day 0). The plates were then incubated further, and measurements were taken at 24, 48, and 72 hours. At each time point, the plates were equilibrated at room temperature, an equal volume of Bright-Glo reagent (Promega, Madison, Wis.) was added, and the plates were read in a LumiCount (Packard BioScience Company, Downers Grove, Ill.) luminometer. For the 384-well plate format, cells were seeded at densities of 1000, 2500, and 5000 cells/well in 30 μL of medium. The remainder of the experiment was carried out as described for 96-well plates. Results were expressed as the mean of three replicate wells.

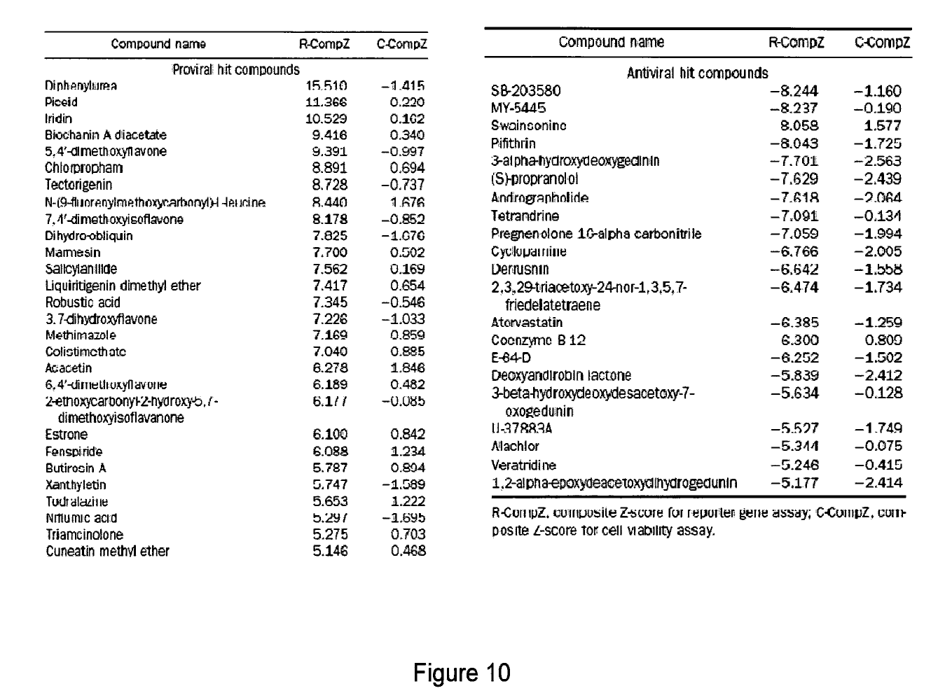

Primary Screening-HTS. Information about the library of known bioactive compounds that was screened and the general automated HTS protocol is available at http://www.broad.harvard.edu/chembio/index.html (see FIG. 10 for list of primary HTS with known bioactives library). Based on the results of optimization experiments in the 384-well plate format, the general HTS protocol was adapted as follows. Unless otherwise indicated, cells were incubated at all times in a humidified environment with 5% CO₂ at 37° C. The assay was initiated by plating 30 μL of medium containing 2000 cells/well into white 384-well opaque-bottom plates (Nunc, Rochester, N.Y.) using an automated plate filler (Bio-Tek uFiller; Winsooki, Vt.) and allowing the cells to adhere for 24 hours. One hundred nL of compound stock solutions in DMSO was transferred from stock plates into the 384-well assay plates using an automated pin-based compound transfer robot (CyBio CyBi-Well vario; Woburn, Mass.). The final compound concentration in each well was estimated to be approximately 10-50 μM, with most compounds at 33 μM. The wells contained 0.33% DMSO by volume. The cells were then incubated for another 48 hours. The luminescent signal from each plate was detected using an automated plate reader (Perkin-Elmer Envision 1; Wellesley, Mass.). This screen was performed in duplicate. For negative controls, entire DMSO-treated control plates were employed, in addition to DMSO-only control wells that were incorporated into each compound assay plate. The cells were assayed for luciferase activity using the Bright-Glo Luciferase assay system (Promega), following the manufacturer's instructions. As a counter-screen, cell viability was assessed using the CellTiterGlo Luminescent cell viability assay (Promega) following the manufacturer's instructions.

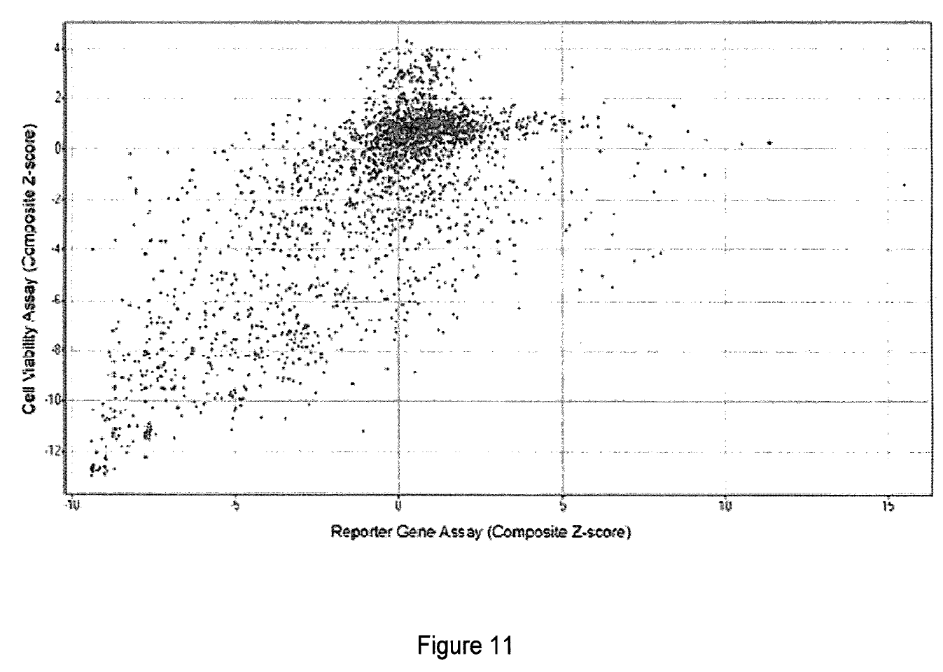

Computational Data Analysis-Primary Screening. For each replicate, a mock-treatment distribution based on the total population of mock (DMSO) controls in that replicate was built (FIG. 11). Each compound was independently assigned a sign (ie, “+” or “−”) Z-score. Z-scores are calculated by dividing each background-subtracted, compound-treated well by the global standard deviation. A global standard deviation of the background-subtracted, mock-treated wells is calculated over the entire experiment. This distribution was determined to be consistent with experimental noise observed under cell-based assay conditions. The resulting collection of continuousvalued Z-scores represents the primary data set to be used for further analysis. The composite Z-score was calculated as a vector projection of each Z-score in duplicate onto an imaginary line of perfect reproducibility. Reproducibility is the cosine of the angle between each Z-score and that imaginary line; it is dimensionless and ranges from −1 to +1. For analyses dependent upon discrete (ie, binned) outcome states, composite Z-score data were further subjected to a threshold that resulted in each measurement being scored as a high- or low-signal outlier, or as a nonoutlier, from the mock-treatment distribution, based on the possibility that the measurement could be explained by assay noise (Pnoise<0.0005). The primary data were analyzed using the commercial software packages Pipeline Pilot (SciTegic, San Diego, Calif.) and SpotFire (SpotFire, Inc., Somerville, Mass.). The means of the negative, DMSO-only controls were considered the zero point. For the bioactives library, compounds were considered hits for inhibiting replication if they had a composite Z-score of <−5.14 in the reporter gene screen, a reproducibility of >0.9 or <−0.9 in that screen, and a composite Z-score of >−2.57 in the cell viability screen. Compounds were considered hits for promoting replication if they had a composite Z-score of >5.14 in the reporter gene screen, a reproducibility of >0.9 or <−0.9 in that screen, and a composite Z-score of <2.57 in the cell viability screen.

HCV Replicon System-Secondary Assays. For validation, OR6 cells stably harboring the full-length genotype 1 replicon, ORN/C-5B/KE9 were used to examine compound activity in a more authentic viral polyprotein context. This replicon was derived from the 1B-2 strain (strain HCV—O, genotype 1b), in which the Renilla luciferase gene is introduced as a fusion protein with neomycin to facilitate the monitoring of HCV replication. This construct contains a tissue culture adaptive mutation in the NS3 region. Cells were cultured in an identical manner to the Huh7/Rep-Feo cells.

Secondary Assays-Hit Validation. Several hits from primary HTS, as well as functionally related compounds, were purchased from Sigma (St. Louis, Mo.), Calbiochem (San Diego, Calif.), and Microsource (Gaylordsville, Conn.). Proviral compounds included (1) corticosteroids-triamcinolone acetonide, prednisolone, dexamethasone, and methylprednisolone, (2) PPAR-gamma ligands-N-(9-fluorenylmethoxy-carbonyl)-L-leucine (Fmoc-Leu) and troglitazone, and (3) coumarins-marmesin, xanthyletin, dihydro-obliquin, warfarin, coumarin, citropen, and dicumarol. Antiviral compounds included (1) PDE inhibitors-MY-5445, trequinsin, zaprinast, and rolipram, (2) calcium channel blockers-tetrandrine, verapamil, nifedipine, diltiazem, and nimodipine, (3) MAPK inhibitors-SB-203580, SB-202190, and PD 98059, as well as a negative control (SB 202474), and (4) HMG-CoA reductase inhibitors-atorvastatin, simvastatin, mevastatin, lovastatin, fluvastatin, and pravastatin. Ten mM of stock solutions of the individual compounds to be tested was prepared in the appropriate solvent (DMSO, ethanol, or H2O, according to the manufacturer's information) and stored at −20° C. Cells were seeded into 96-well plates at a density of 2000 cells/well in 100 μL of medium. The cells were incubated for 24 hours at 37° C. to obtain the optimal level of adherence. Solutions of candidate hit compounds were added to wells to achieve final concentrations of 0.1, 1, 10, 50, and 100 μM. The final concentration of DMSO or ethanol in every well was 1% or less by volume. Mock solutions were used as a negative control. PEG-IFN and ribavirin were used as positive controls at various concentrations, alone and in combination. The plates were then incubated at 37° C. with 5% CO₂ for 48 hours before they were analyzed. Luminescent signal was generated using the Renilla luciferase assay kit (Promega) according to the manufacturer's instructions. Signal was then detected using a LumiCount (Packard BioScience Company) luminometer. Cell viability was assessed using CellTiter-Glo (Promega), following the manufacturer's instructions. All experiments were performed in triplicate.

Secondary Assays-Data Analysis. Values were presented as a percentage of mock treated control, which was arbitrarily set at 100%. Data were expressed as the mean (±) SD. Results were analyzed using a paired t test to determine the significance of observed differences between the values of control and individual concentrations and were considered significant if the P values were less than 0.05. Synergy calculations were performed using CalcuSyn (Biosoft; Cambridge, England).

Optimization of Huh-7/Rep-Feo Cells for the 384-Well Plate Format. HCV replication in the subgenomic replicon cell model was tightly coupled to host cell growth conditions. Experimentally, HCV RNA replication in the subgenomic replicon cell increased progressively over time, followed by a sharp decline when the cells reached 70% confluence. There was a good linear relationship between cell number and luciferase signal in test tube. Inasmuch as the wells of microplates have a small culturable surface, the Huh7/Rep-Feo cells were optimized for both the 96-well and 384-well plate formats using PEG-IFN as a positive control. For 96-well plates, the signal-to-background (S/B) ratio was very high (over 100) when cells were incubated at a concentration of 5000 cells/well at culture day 0. The S/B ratio progressively increased over time, peaking at culture day 2 and then decreased from culture day 3 onward, when cells reached over 70% confluence. The antiviral activity of PEG-IFN progressively increased over time. On the other hand, although the initial S/B ratio at an inoculation concentration of 10,000 cells/well was higher than at 5000 cells/well, there was no significant increase of the S/B ratio over time. For 384-well plates, the S/B ratio at an inoculation concentration of 1000 cells/well progressively increased over time, with a large standard deviation. The S/B ratio at an inoculation concentration of 5000 cells/well peaked at culture day 1, when an optimum level of confluence was reached. The S/B ratio at an inoculation concentration of 2500 cells/well was ideal, with the optimal S/B ratio achieved at culture day 2 (FIG. 9). Therefore, 2500 cells/well at culture day 2 was selected for subsequent studies.

Primary Screening (HTS) Result. FIG. 11 shows a graphical representation of the primary HTS results. Many compounds appeared to have strong antiviral activity when the luciferase reporter gene assay alone was considered. When these results were analyzed in conjunction with those of the cell viability assay, however, most of the potential antiviral hit compounds were cytotoxic and, therefore, false positives. For that reason, it is imperative to perform the primary HTS as a 2-dimensional assay with both the level of HCV replication and cell viability measurements, in order to minimize confounding from increased luciferase signals due to increased cell titer and decreased luciferase signals due to decreased cell titer. Using the data analysis and hit selection criteria outlined above in Materials and Methods, we identified 21 antiviral compounds that inhibited HCV replication and 28 proviral compounds that increased HCV replication (FIG. 10). The respective hit rates of 0.8% and 1.1% are consistent with hit rates for other biological screens performed using this library. Proviral compounds included steroids (estrone, triamcinolone), coumarins (xanthylentin, dihydrobliquin, and marmesin), flavones, and a PPAR-gamma ligand (N-9-fluorenylmethoxycarbonyl-L-leucine). Antiviral compounds included an HMG-CoA reductase inhibitor (atorvastatin), a beta-adrenergic blocker (propranolol), a calcium channel blocker (tetrandrine), a phosphodiesterase (PDE) inhibitor (MY-5445), and a p38 MAP kinase inhibitor (SB 203580). The finding of antiviral activity associated with the HMG-CoA reductase inhibitor was of particular interest, as the HMG-CoA reductase inhibitor lovastatin has recently been shown to exhibit anti-HCV activity. Although corticosteroids have been assumed to increase HCV replication by means of host immunosuppression, they have not been reported to be a specific proviral agent for HCV independent of their general immunosuppressive activity.

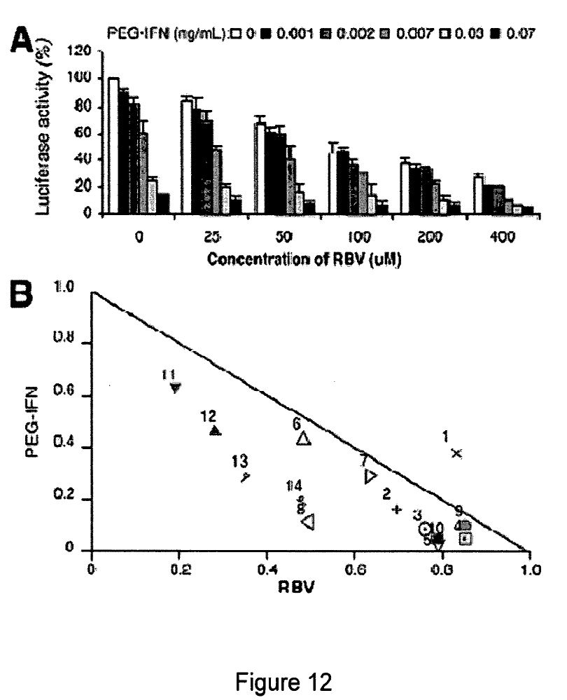

Anti-HCV Activity of PEG-IFN and Ribavirin in the OR6Replicon System. We tested PEG-IFN and ribavirin at various concentrations, alone and in combination, as it has been reported that OR6 cells bearing a genome-length HCV RNA replicon were sensitive to these agents.9 The IC50 of PEG-IFN was between 0.007 and 0.03 ng/mL. The IC50 of ribavirin was between 50 μM and 100 μM (FIG. 12A). The combination of ribavirin with PEG-IFN showed synergy (FIG. 12B). These results demonstrate that HCV RNA replication in OR6 cells is highly sensitive to PEG-IFN, ribavirin, and a combination of both agents.

Hit Validation. Several proviral and antiviral hit compounds identified in the primary screen were selected for further validation on the basis of commercial availability and clinical interest. In order to examine compound activity in a more authentic viral polyprotein context, the validation assays were carried out using the OR6 full-length genotype 1b replicon. Multiple concentrations of each compound were used in order to generate an adequate dose-response curve. In addition to the actual hit compounds themselves, other compounds from the relevant compound classes were subjected to secondary validation assays.

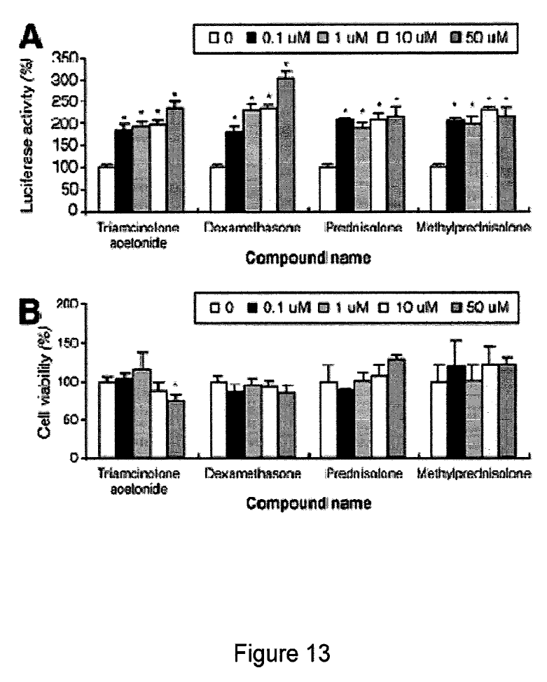

Proviral compounds. Triamcinolone was confirmed to increase HCV replication in the full-length HCV replicon system (FIGS. 13A and 13B). Other corticosteroids (prednisolone, dexamethasone, and methylprednisolone) also increased HCV replication (FIGS. 13A and 13B). The PPAR gamma ligand, N-(9-fluorenylmethoxycarbonyl)-L-leucine, which was identified as a proviral hit in the primary screen, showed mild proviral activity. Troglitazone showed proviral activity at 1 and 10 μM. The decreased luciferase signal at 50 μM and above was due to cytotoxicity. Clofibrate, a PPAR alpha ligand, did not show a proviral effect. The coumarin compounds also did not demonstrate any significant proviral activity.

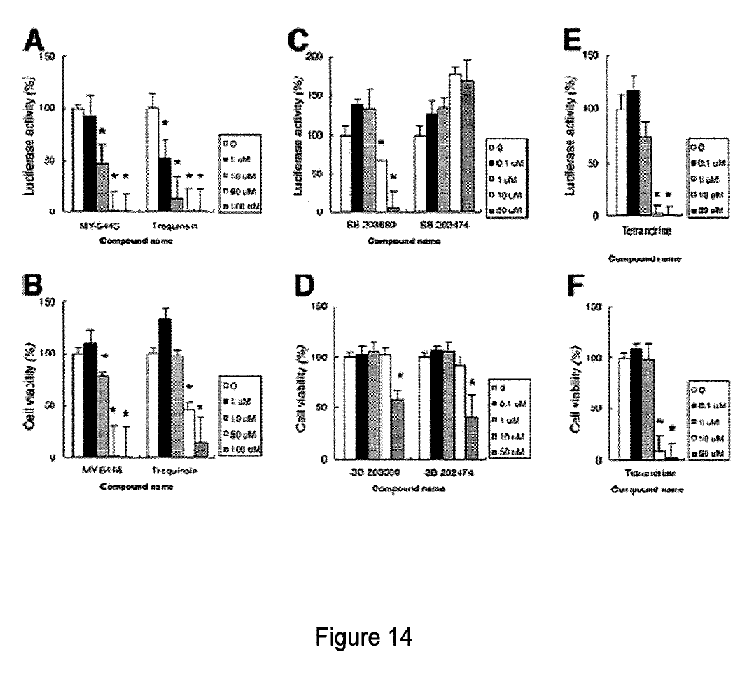

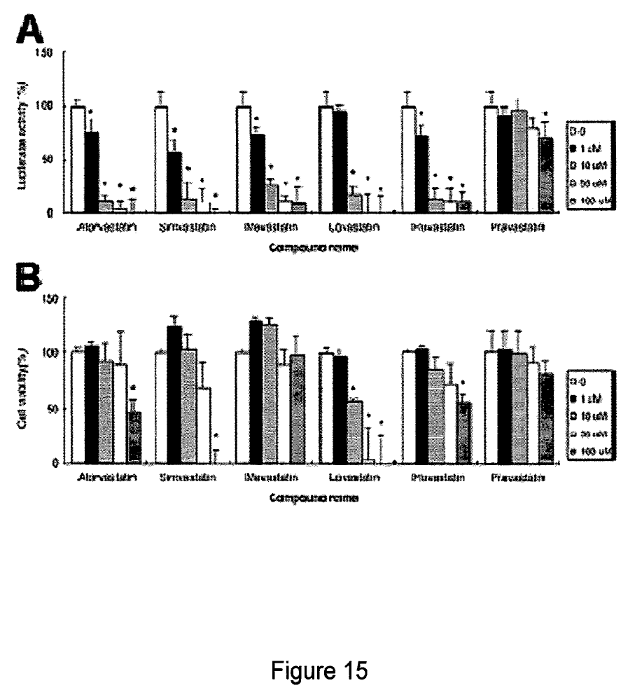

Antiviral compounds. Although the PDE inhibitor, MY5445, decreased the luciferase signal in a dose-related manner, it displayed significant cytotoxicity. Another PDE inhibitor in the bioactives library, trequinsin, was not identified as a hit in the primary screen, where it was tested at a concentration of 33 μM and found to be cytotoxic. It did, however, display antiviral activity at the lower concentrations of 1 and 10 μM in the validation assay, with significant cytotoxicity only at the higher concentrations of 50 and 100 μM (FIGS. 14A and 14B). The p38 MAPK inhibitor, SB 203580, exhibited antiviral activity at a concentration of 10 μM and cytotoxicity at higher concentrations (FIGS. 14C and 14D). Other MAPK inhibitors, such as SB 202109, were inactive in both the primary screen and in the secondary assay. The antiviral effect of the calcium channel blocker, tetrandrine, could not be evaluated because of cytotoxicity in the secondary assay (FIGS. 14E and 14F). Other calcium channel blockers, such as verapamil and nifedipine, did not exhibit antiviral activity in either the primary screen or the validation assay. Strikingly, each of the HMG-CoA reductase inhibitors, except for pravastatin, significantly decreased HCV replication in a dose-related manner, with IC50 values between 1 and 10 μM. Atorvastatin, simvastatin, and fluvastatin demonstrated strong antiviral effects. Lovastatin and mevastatin were weakly inhibitory. Lovastatin was significantly cytotoxic at 10, 50, and 100 μM. Mevastatin was not cytotoxic. Pravastatin showed very weak antiviral activity, with only 30% inhibition at 100 μM (FIGS. 15A and B).

### Example 2

### Identification of Novel Epoxide Inhibitors of HCV Replication Using High Throughput Screening

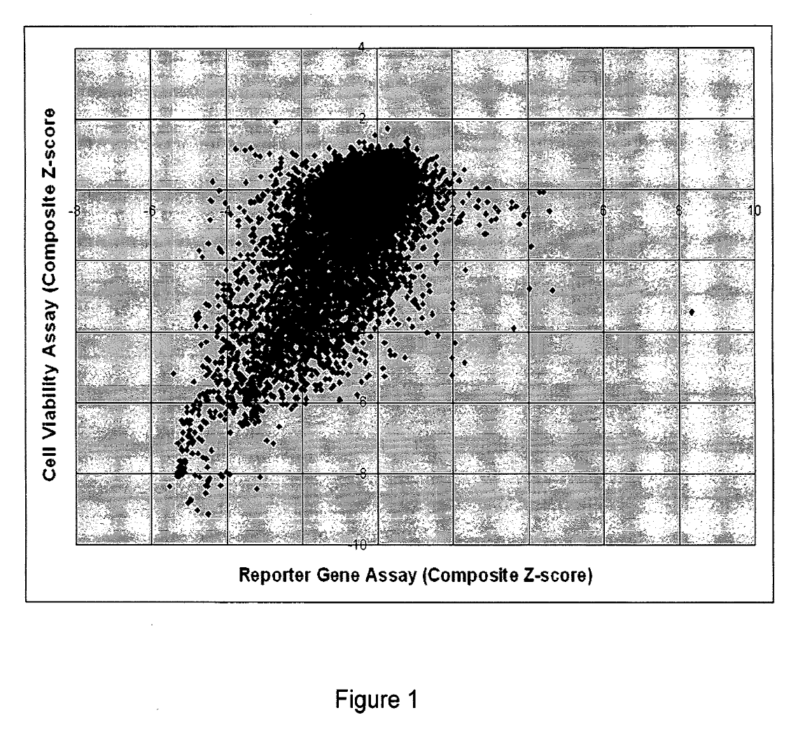

Compounds of the DOS set at the Broad Institute Chemical Biology Platform HTS facility were assayed, in order to discover novel regulators of HCV replication (Kim et al., Gastroenterology (2007) 132:311-320). FIG. 1 shows a graphical representation of the primary HTS results. Many compounds appeared to have strong antiviral activity when the luciferase reporter gene assay alone was considered. When these results were analyzed in conjunction with those of the cell viability assay, however, most of the potential antiviral hit compounds were false positives due to their cytotoxicity. It is therefore generally more useful to perform the primary HTS as a 2-dimensional assay with both the level of HCV replication and cell viability measurements. This minimizes confounding from increased luciferase signals due to increased cell titer and decreased luciferase signals due to decreased cell titer.

Using the data analysis and hit selection criteria outlined in Methods, we identified 21 proviral compounds that increased HCV replication (Table 1), and 41 antiviral compounds that inhibited HCV replication (Table 2). The respective hit rates of 0.5% and 0.3% are consistent with hit rates for other biological screens performed with DOS libraries.

TABLE 1
Pro-viral hit compounds

| Compound name | R-CompZ^{a} | C−CompZ^{b} |
|---|---|---|
| BUCMLD-B8 | 8.3346 | −3.4524 |
| BUCMLD-B8A6 | 4.6372 | −2.8306 |
| FPA1_000056 | 4.5527 | −0.5996 |
| A32B9C3 | 4.395 | −0.0793 |
| FPA1_000059 | 4.0764 | −1.5987 |
| FPA1_000282 | 4.0267 | −2.7919 |
| FPA1_000165 | 3.7617 | −0.9662 |
| BUCMLD-JRG-2-103 | 3.6144 | −3.9077 |
| FPA1_000229 | 3.5665 | −0.3545 |
| A10B9C3 | 3.2114 | −0.6043 |
| FPA1_000110 | 3.1479 | −0.8117 |
| SUGA2_000176 | 3.0224 | −0.8583 |
| FPA1_000055 | 2.9296 | −0.3329 |
| BUCMLD-JRG-1-178 | 2.8765 | −1.2499 |
| UGISS-323 | 2.8718 | −0.6788 |
| SUGA2_000044 | 2.6917 | −0.9888 |
| FPA1_000310 | 2.6786 | −0.2858 |
| FPA1_000028 | 2.6667 | −0.5127 |
| JMM3-47 | 2.6215 | 0.0511 |
| FPA1_000254 | 2.6178 | −0.4767 |
| BUCMLD-B8 | 8.3346 | −3.4524 |
| BUCMLD-B8A6 | 4.6372 | −2.8306 |
| FPA1_000056 | 4.5527 | −0.5996 |
| A32B9C3 | 4.395 | −0.0793 |
| FPA1_000059 | 4.0764 | −1.5987 |
| FPA1_000282 | 4.0267 | −2.7919 |
| FPA1_000165 | 3.7617 | −0.9662 |
| BUCMLD-JRG-2-103 | 3.6144 | −3.9077 |
| UGISS-323 | 2.8718 | −0.6788 |
| SUGA2_000044 | 2.6917 | −0.9888 |
| FPA1_000310 | 2.6786 | −0.2858 |
| FPA1_000028 | 2.6667 | −0.5127 |
| JMM3-47 | 2.6215 | 0.0511 |
| FPA1_000254 | 2.6178 | −0.4767 |

^{a}R-CompZ, compositeZ score for reporter gene assay;
^{b}C-CompZ, compositeZ score for cell viability assay

TABLE 2
Antiviral hit compounds

| Compound name | R−CompZ^{a} | C−CompZ^{b} |
|---|---|---|
| BUCMLD-B10A11 | −4.7324 | −1.7178 |
| BUCMLD-B10A3 | −4.6336 | −1.8907 |
| BUCMLD-B10A1 | −4.4774 | −1.5319 |
| BEA2_000182 | −4.4448 | −0.8538 |
| HUM-SAH23 | −4.194 | −1.6526 |
| HUM-SAH25 | −4.1269 | −1.7616 |
| 050/Coumarine010 | −3.8829 | −0.6917 |
| BUCMLD-B10A8 | −3.7511 | 1.1582 |
| 048/Coumarine008 | −3.6894 | −0.8703 |
| SM_A5B5_2P118 | −3.6565 | −0.4963 |
| RTE2_000765 | −3.5389 | −0.5956 |
| BUCMLD-B10A14 | −3.5069 | 1.128 |
| BUCMLD-XL-189 | −3.4882 | 0.2217 |
| SM_A4B6_2P123 | −3.4419 | −0.795 |
| SM_A1B5_2P24 | −3.38 | −0.856 |
| FPA1_000158 | −3.3644 | −0.3596 |
| BUCMLD-B10A7 | −3.3561 | 1.1731 |
| BEA2_000178 | −3.2744 | −0.3445 |
| SM_A1B2_1P32 | −3.252 | 0.9925 |
| BUCMLD-B13A1 | −3.1824 | −0.2035 |
| BUCMLD-NTM-EN2-67A | −3.167 | −0.9198 |
| FPA1_000202 | −3.1496 | −0.7982 |
| SM_A6B5_2P100 | −3.1341 | −0.2965 |
| BUCMLD-B10A5 | −3.0849 | 0.3574 |
| BUCMLD-B13A2 | −3.0733 | −0.8097 |
| BUCMLD-XL-184 | −3.0142 | −0.1464 |
| FPA1_000357 | −2.8803 | −0.7677 |
| FPA1_000381 | −2.8759 | −0.5599 |
| BUCMLD-B10A13 | −2.8629 | 1.0278 |
| BUCMLD-XL-130 | −2.8179 | 0.4015 |
| FPA1_000277 | −2.8135 | −0.409 |
| II_G03 | −2.7674 | −0.9892 |
| BEA2_000173 | −2.7556 | 0.1421 |
| SM_A5B4_2P126 | −2.7406 | −0.232 |
| SM_A5B2_2P142 | −2.7176 | 0.5666 |
| BUCMLD-B10A10 | −2.7028 | 1.9213 |
| SM_A7C2_2P155 | −2.6813 | 0.6028 |
| HUM-SAH24 | −2.664 | −0.4661 |
| FPA1_000155 | −2.6456 | −0.7045 |
| BUCMLD-XL-190 | −2.5928 | 0.4558 |
| SM_A5B3_2P141 | −2.5923 | 0.6345 |

^{a}R-CompZ, compositeZ score for reporter gene assay;
^{b}C-CompZ, compositeZ score for cell viability assay

Optimization of Huh-7/Rep-Feo cells for the 384-well plate format: Cells were propagated in Dulbecco's Modified Eagle's medium (DMEM) containing 10% fetal bovine serum (FBS) supplemented with 1% penicillin-streptomycin, and 500 μg of Geneticin (Invitrogen Corp., Carlsbad, Calif.)/ml. Cells were cultured in a 37° C., 5% CO₂-humidified incubator for all experiments. To decrease day-to-day variability in the assay, a large homogenous population of subconfluent cells was passaged so that a similar lot of cells could be used throughout the HTS assay. The Huh7/Rep-Feo cells were first optimized for the 96-well plate format. Peginterferon-alfa-2b (PEG-Intron; Schering Corp., Kenilworth, N.J.) was used as a positive control for inhibition. Cells were seeded at densities of 5,000 and 10,000 cells/well in 100 μl of medium in 96-well plates. The cells were allowed to attach overnight (˜24 hr) before addition of PEG-IFN at various concentrations (day 0). The plates were then incubated further, and measurements were taken at 24, 48, and 72 hr. At each time point, the plates were equilibrated at room temperature, an equal volume of Bright-Glo reagent (Promega, Madison, Wis.) was added, and the plates were read in a LumiCount™ (Packard BioScience Company, Downers Grove, Ill.) luminometer. For the 384-well plate format, cells were seeded at densities of 1,000, 2,500, and 5,000 cells/well in 30 μl of medium. The remainder of the experiment was carried out as described for 96-well plates. Results were expressed as the mean of three replicate wells. HCV replication in the subgenomic replicon cell model was tightly coupled to host cell growth conditions. Experimentally, HCV RNA replication in the subgenomic replicon cell increased progressively over time, followed by a sharp decline when the cells reached 70% confluence. There was a good linear relationship between cell number and luciferase. Inasmuch as the wells of microplates have a small culturable surface, the Huh7/Rep-Feo cells were optimized for both the 96-well and 384-well plate formats using PEG-IFN as a positive control. For 96-well plates, the signal-to-background (S/B) ratio was very high (over 100) when cells were incubated at a concentration of 5,000 cells/well at culture day 0. The S/B ratio progressively increased over time, peaking at culture day 2 and then decreased from culture day 3 onwards, when cells reached over 70% confluence. The antiviral activity of PEG-IFN progressively increased over time. On the other hand, although the initial S/B ratio at an inoculation concentration of 10,000 cells/well was higher than at 5,000 cells/well, there was no significant increase of the S/B ratio over time. For 384-well plates, the S/B ratio at an inoculation concentration of 1,000 cells/well progressively increased over time, with a large standard deviation. The S/B ratio at an inoculation concentration of 5,000 cells/well peaked at culture day 1, when an optimum level of confluence was reached. The S/B ratio at an inoculation concentration of 2,500 cells/well was ideal, with the optimal S/B ratio achieved at culture day 2. 2,500 cells/well at culture day 2 was selected for subsequent studies. To decrease day-to-day variability in the assay, a large homogenous population of subconfluent cells was passaged so that a similar lot of cells could be used throughout the HTS assay.

Primary Screening—HTS: Unless otherwise indicated, cells were incubated at all times in a humidified environment with 5% CO₂ at 37° C. The assay was initiated by plating 30 μl of medium containing 2,000 cells/well into white 384-well opaque-bottom plates (Nunc; Rochester, N.Y.) using an automated plate filler (Bio-Tek μFiller; Winsooki, Vt.) and allowing the cells to adhere for 24 hours. 100 nl of compound stock solutions in DMSO were transferred from stock plates into the 384-well assay plates using an automated pin-based compound transfer robot (CyBio CyBi-Well vario; Woburn, Mass.). The final compound concentration in each well was estimated to be approximately 10-50 μM, with most compounds at 33 μM. The wells contained 0.33% DMSO by volume. The cells were then incubated for another 48 hours. The luminescent signal from each plate was detected using an automated plate reader (Perkin-Elmer Envision 1; Wellesley, Mass.). This screen was performed in duplicate. Peginterferon-alfa-2b (PEG-Intron; Schering Corp., Kenilworth, N.J.) was used as a positive control for inhibition. For negative controls, entire DMSO-treated control plates were employed, in addition to DMSO-only control wells that were incorporated into each compound assay plate. The cells were assayed for luciferase activity using the Bright-Glo Luciferase assay system (Promega; Madison, Wis.), following the manufacturer's instructions. As a counter-screen, cell viability was assessed using the CellTiterGlo Luminescent cell viability assay (Promega; Madison, Wis.), following the manufacturer's instructions.

Computational Data Analysis—Primary Screening: For each replicate, a mock-treatment distribution based on the total population of mock (DMSO) controls in that replicate was built. Each compound was independently assigned a signed (i.e., “+” or “−”) Z-score. Z-scores are calculated by dividing each background-subtracted, compound-treated well by the global standard deviation. A global standard deviation of the background-subtracted, mock-treated wells is calculated over the entire experiment. This distribution was determined to be consistent with experimental noise observed under cell-based assay conditions. The resulting collection of continuous-valued Z-scores represents the primary dataset to be used for further analysis. The composite Z-score was calculated as a vector projection of each Z-score in duplicate onto an imaginary line of perfect reproducibility. Reproducibility is the cosine of the angle between each Z-score and that imaginary line; it is dimensionless and ranges from −1 to +1. For analyses dependent upon discrete (i.e., binned) outcome states, composite Z-score data were further subjected to a threshold that resulted in each measurement being scored as a high- or low-signal outlier, or as a non-outlier, from the mock-treatment distribution, based on the possibility that the measurement could be explained by assay noise (Pₙₒᵢₛₑ<0.0005).

The primary data were analyzed using the commercial software packages Pipeline Pilot (SciTegic; San Diego, Calif.) and SpotFire (SpotFire, Inc.; Somerville, Mass.). The means of the negative, DMSO-only controls were considered the zero point. Compounds were considered hits for inhibiting replication if they had a composite Z-score of <−2.57 in the reporter gene screen, a reproducibility of >0.9 or <−0.9 in that screen, and a composite Z-score of >−2.00 in the cell viability screen. Compounds were considered hits for promoting replication if they had a composite Z-score of >2.50 in the reporter gene screen, a reproducibility of >0.9 or <−0.9 in that screen, and a composite Z-score of <1.00 in the cell viability screen.

HCV Replicon System—Secondary Assays: For replicon validation, OR6 cells stably harboring the full-length genotype 1 replicon, ORN/C-5B/KE were used (Burke J. Am. Chem. Soc. (2004) 126:14095-14104). This replicon was derived from the 1B-2 strain (strain HCV—O, genotype 1b), in which the Renilla luciferase gene is introduced as a fusion protein with the Neomycin resistance cassette to facilitate the monitoring of HCV replication. This construct contains a tissue culture adaptive mutation in the NS3 region. Cells were cultured in an identical manner to the Huh7/Rep-Feo cells.

Secondary Assays—Hit Validation: 10 mM of stock solutions of the individual compounds to be tested were prepared in DMSO and stored at −20° C. Cells were seeded into 96-well plates at a density of 2,000 cells/well in 100 μl of medium. The cells were incubated for 24 hours at 37° C. to obtain the optimal level of adherence. Solutions of candidate hit compounds were added to wells to achieve varying final concentrations between 0.01 and 100 μM. The final concentration of DMSO or ethanol in every well was 1% or less by volume. Mock solutions were used as a negative control. Peginterferon-alfa-2b (PEG-Intron; Schering Corp.; Kenilworth, N.J.) and ribavirin (Sigma; St. Louis, Mo.) were used as positive controls at various concentrations, alone and in combination. The plates were then incubated at 37° C. with 5% CO₂ for 48 hours before they were analyzed. Luminescent signal was generated using the Renilla luciferase assay kit (Promega; Madison, Wis.) according to the manufacturer's instructions. Signal was then detected using a LumiCount™ (Packard BioScience Company; Downers Grove, Ill.) luminometer. Cell viability was assessed using CellTiter-Glo (Promega; Madison, Wis.), following the manufacturer's instructions. All experiments were performed in triplicate. Values were presented as a percentage of mock treated control, which was arbitrarily set at 100%. Data were expressed as the mean±SD.

Secondary Assays—Data Analysis: Values were presented as a percentage of mock treated control, which was arbitrarily set at 100%. Data were expressed as the mean±SD. Results were analyzed using a paired t test to determine the significance of observed differences between the values of control and individual concentrations and were considered significant if the p values were less than 0.05.

Among the DOS libraries with multiple hits that were found to increase HCV replication were the JMM libraries (Mitchell and Shaw, Angew. Chem. Int.l Ed (2006) 45:1722-1726), and the FPA libraries (Chen et al., J. Am. Chem. Soc. (2003) 125:10174-10175), as well as a library of flavone derivatives from the CMLD-BU. Among the DOS libraries with multiple hits that were found to decrease HCV replication were the SM libraries (Lo et al., J. Am. Chem. Soc. (2004) 126: 16077-16086), the FPA-SpOx libraries (Chen et al., J. Am. Chem. Soc. (2003) 125:10174-10175), and the BUCMLD epoxyquinol libraries (Lei et al., J. Org. Chem. (2005) 6474-6483; Su et al., Org. Lett. (2005) 7:2751-2754).

In the analysis of the antiviral hit compounds from the DOS Set, a striking finding was that 20 of the 41 compounds contained an epoxide moiety. Moreover, the most potent of these compounds were epoxides (Tables 3 and 4). Further analysis revealed that these epoxides came from only the SM libraries and BUCMLD epoxyquinol libraries.

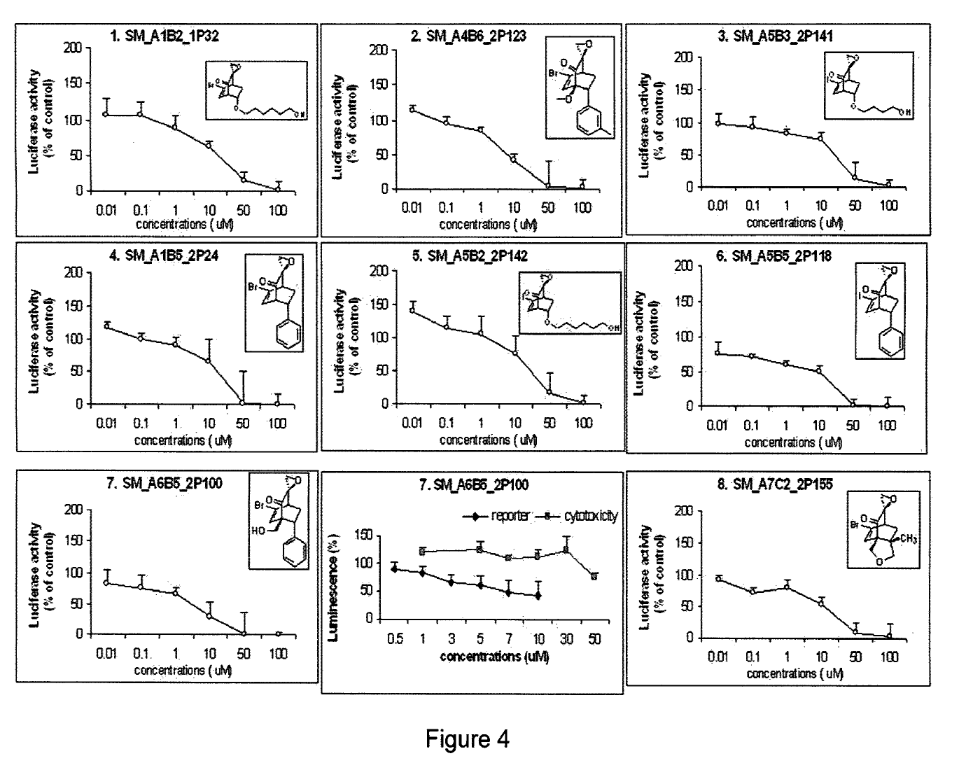

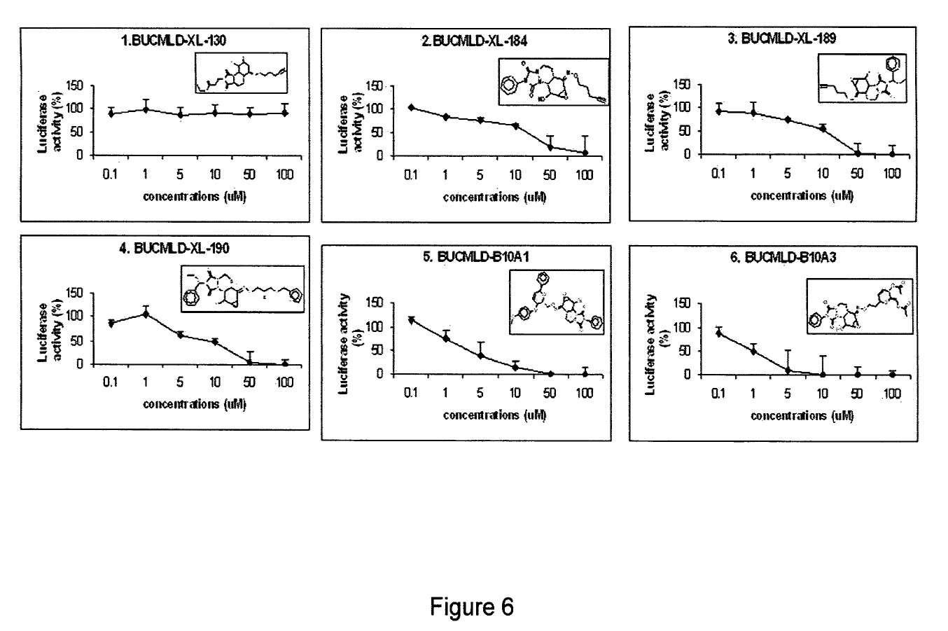

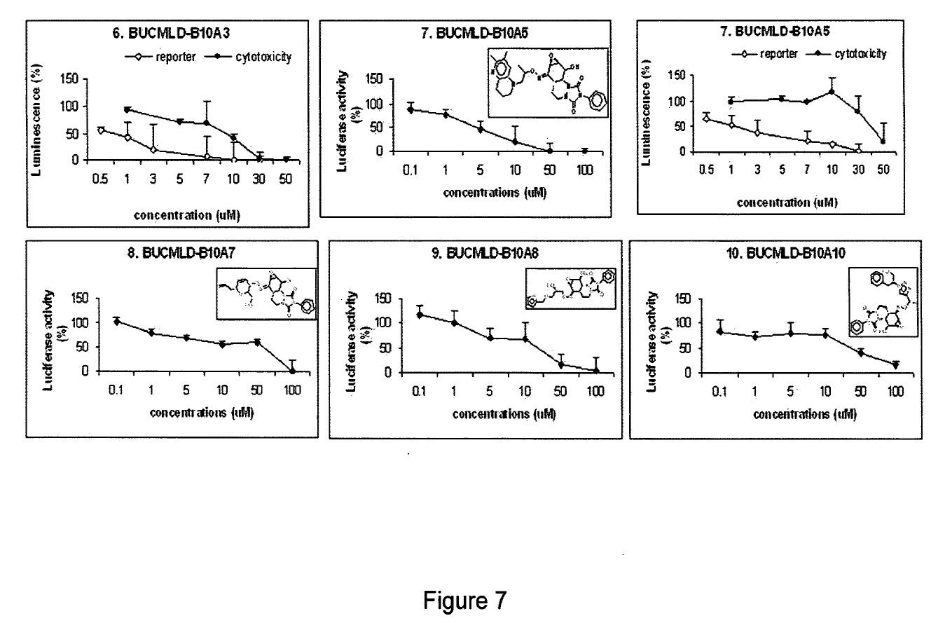

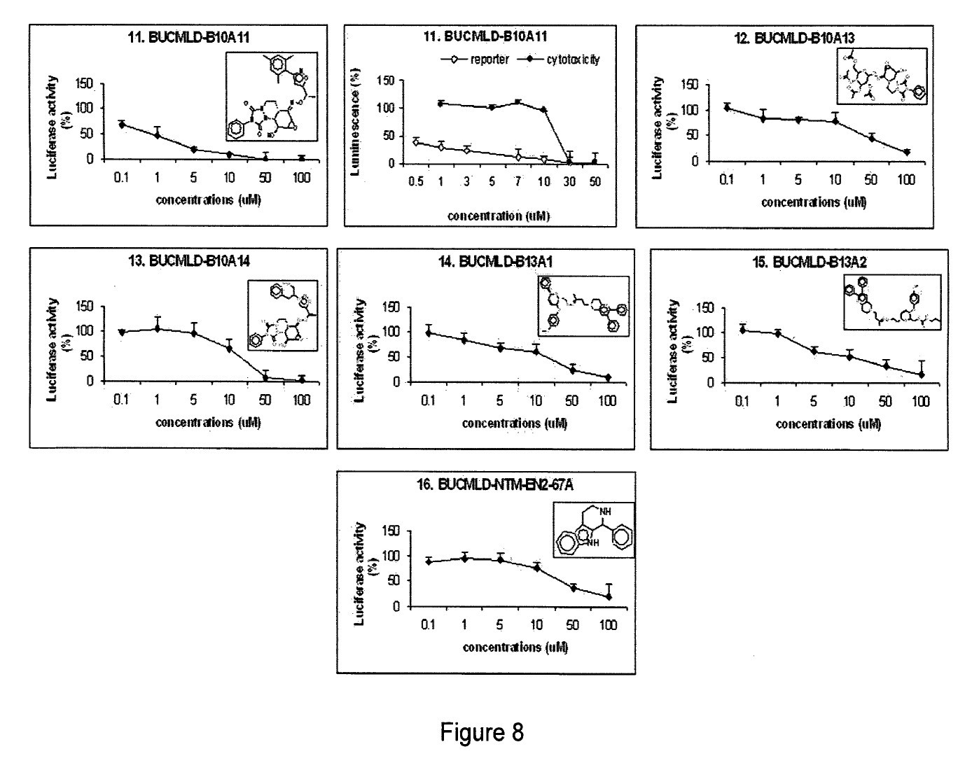

In order to examine compound activity in a more authentic viral polyprotein context, the validation assays of the SM and BUCMLD epoxyquinol libraries were carried out using the OR6 full-length genotype 1b replicon (Ikeda et al., Biochem. Biophys. Res. Commun (2005) 329:1350-1359). Multiple concentrations of each compound were used in order to generate an adequate dose-response curve (FIGS. 4 and 6-8). SM_A6B5_{—}2P100 was the most active member of the SM library, with an IC50 of about 7 μM, with no significant cytotoxic effects at concentrations of 30 μM and below. BUCMLD-B10A11, the most potent member of the BUCMLD epoxyquinol library, had an IC50<500 nM, with no observed cytotoxicity at concentrations of 10 μM and below. BUCMLD-B10A3 and BUCMLD-B10A5 were also quite potent, with 500 nM<IC50<1 μM, and the latter with an IC50 of approximately 1 μM. BUCMLD-B10A3 exhibited cytotoxic effects above 7 μM, while BUCMLD-B10A5 did not exhibit cytotoxicity until 30 μM.

SAR analysis of the hit compounds from the SM library reveals which of the structural elements are most important for antiviral activity. Comparing SM_A5B5_{—}2P118 to SM_A1B5_{—}2P24, iodinated compounds are slightly more active than brominated ones. Comparing SM_A5B5_{—}2P118 to SM_A5B3_{—}2P141 and SM_A5B2_{—}2P142, SM_A4B6_{—}2P123 and SM_A1B5_{—}2P24 to SM_A1B2_{—}1P32, and SM_A4B6_{—}2P123 and SM_A6B5_{—}2P100 to SM_A7C2_{—}2P155, compounds with a phenyl substituent are more active than those with aliphatic chains. Finally, the most active compounds, SM_A4B6_{—}2P123 and SM_A6B5_{—}2P100, have a bridgehead substituent.

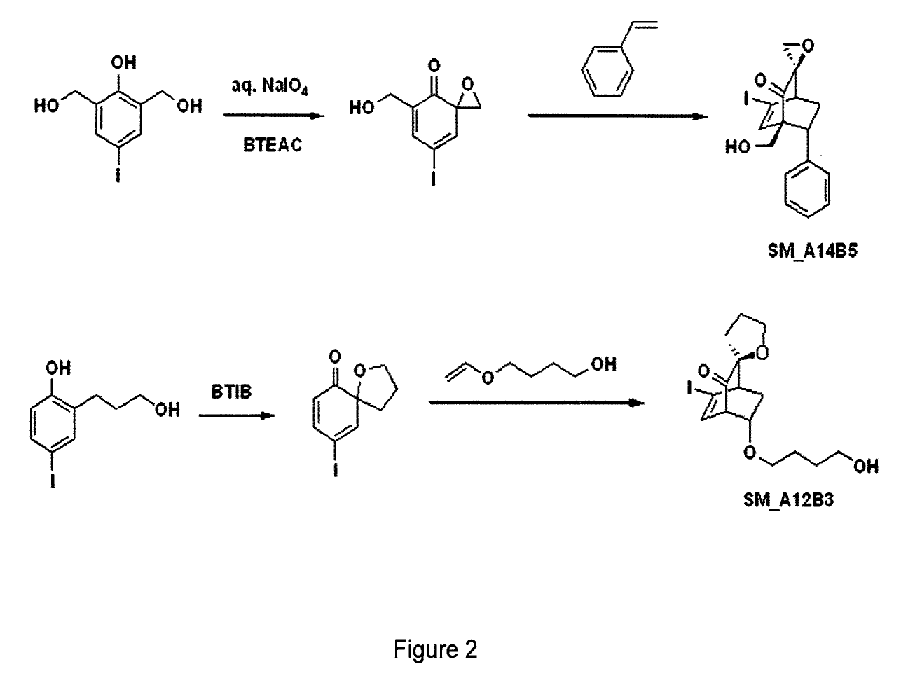

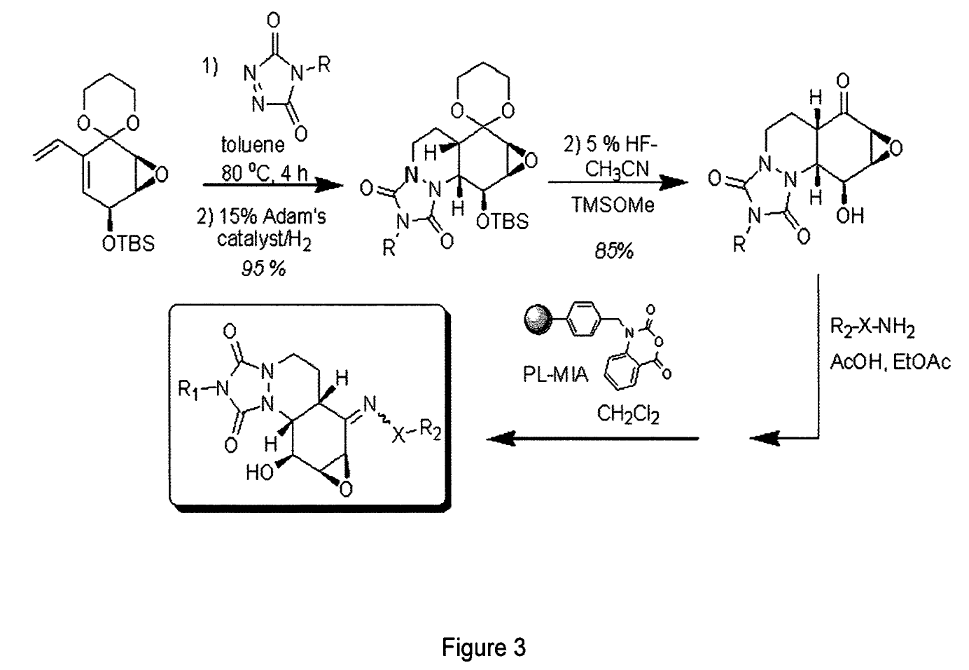

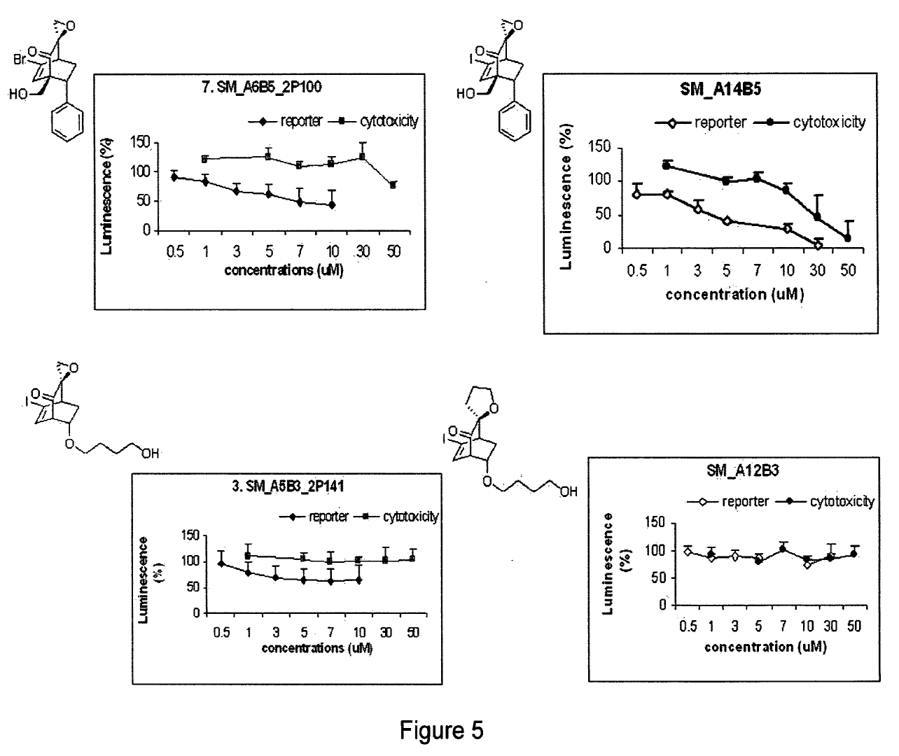

SM_A14B5, which incorporates a iodine, a phenyl substituent, and a bridgehead substituent, was therefore synthesized, as it was reasoned to be the most active SM library compound (FIG. 2). Indeed, SM_A14B5 had an IC₅₀ of approximately 3.5 μM, which is about half that of SM_A6B5_{—}2P100, the most active SM compound in the original DOS Set. Furthermore, it only began to show cytotoxic effects above 30 μM (FIG. 5).

SM_A12B3, an analog of SM_A5B3_{—}2P141, which bears a tetrahydrofuran moiety in place of an epoxide, was synthesized to further test the hypothesis that the epoxide moiety is essential for antiviral activity. SM_A12B3 had negligible antiviral activity. On the other hand, SM_A5B3_{—}2P141 displayed modest antiviral activity, with 10 μM<IC₅₀<50 μM. Other analogs of SM compounds bearing tetrahydrofuran rings in place of epoxides showed similar attenuation of antiviral activity relative to their parent compounds.

Exemplary SM Compounds

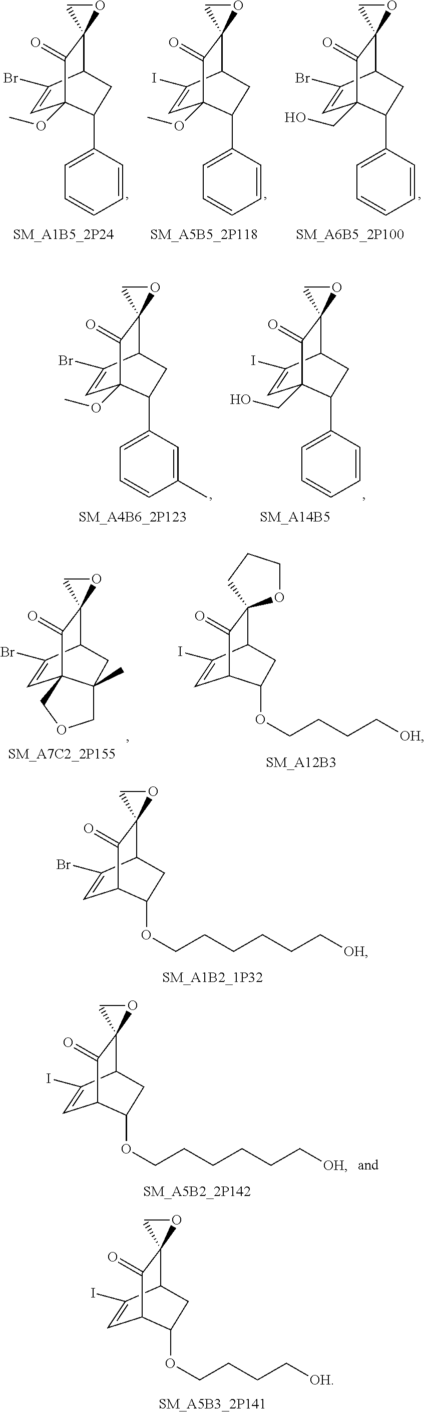

Analyzing the BUCMLD compounds, those compounds that bear an epoxide moiety are, in general, more potent antivirals than those that do not, such as BUCMLD-XL-130, BUCMLD-B13A1, BUCMLD-B13A2, and BUCMLD-NTM-EN2-67A. Further screening of the epoxide containing BUCMLD compounds reveals that the urazole-containing constituents of this library demonstrate more potent anti-HCV activity.

Exemplary BUCMLD (Urazole-Containing) Compounds

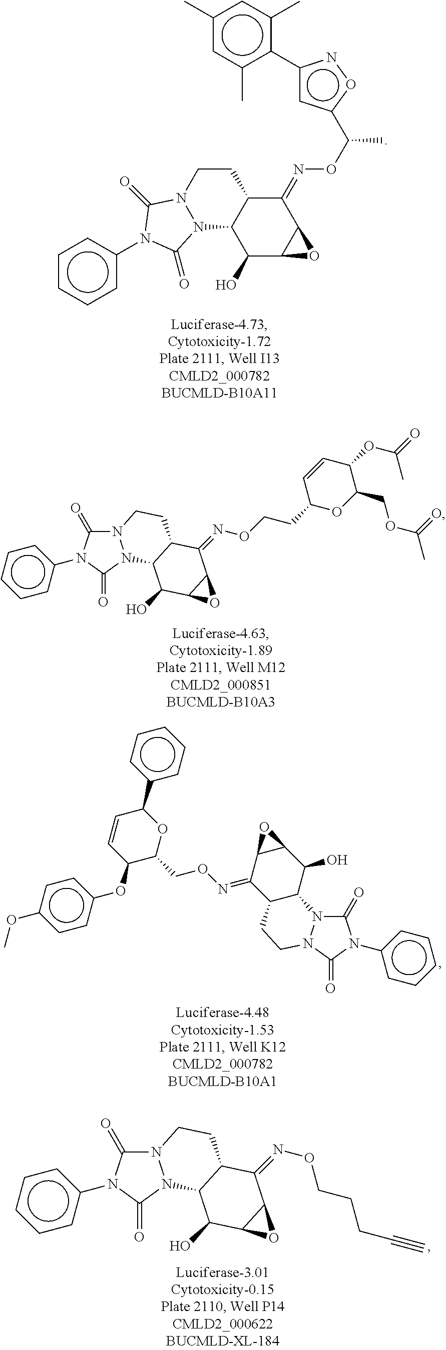

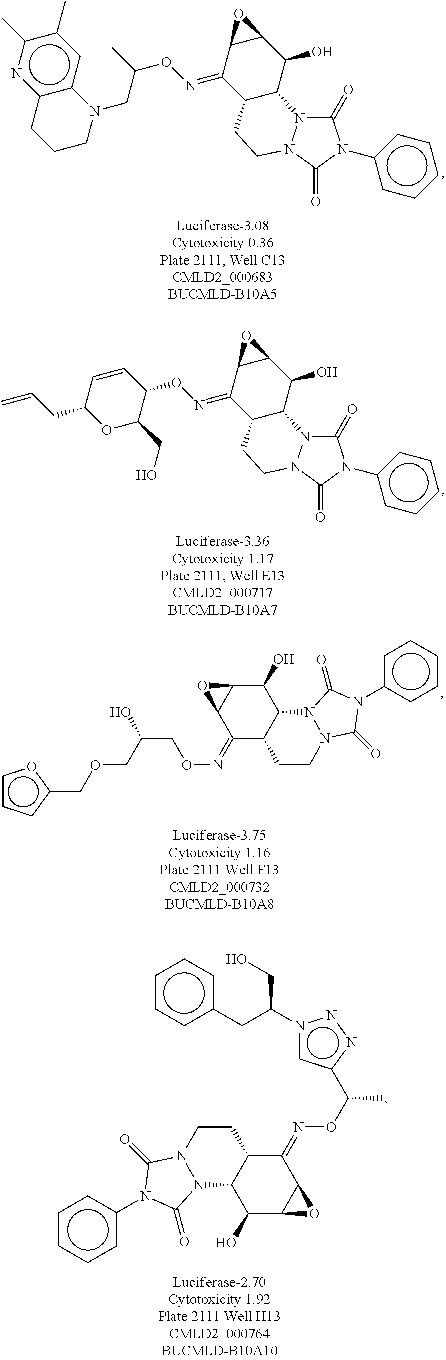

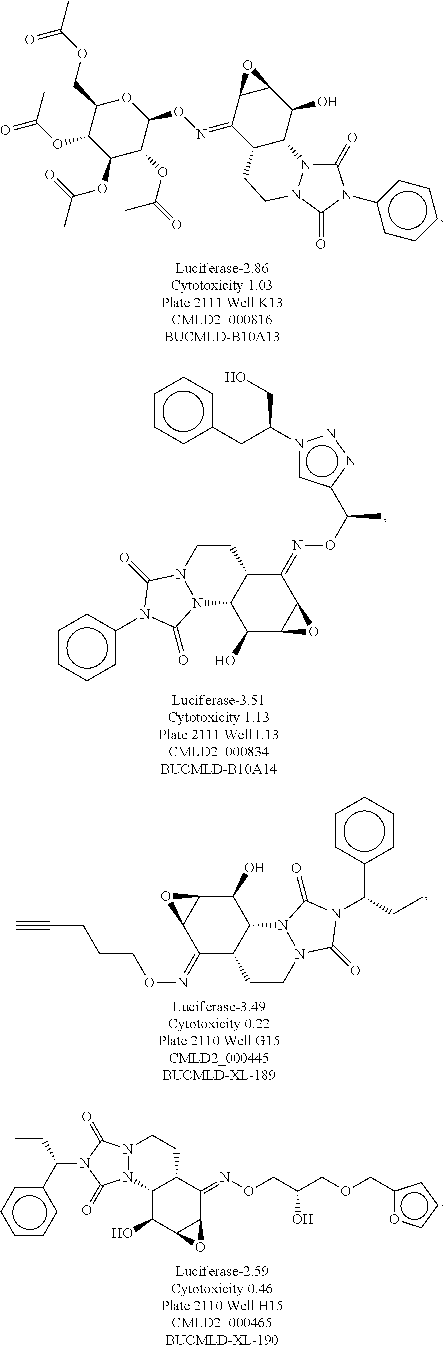

### Example 3

### Synthesis of Exemplary Compounds

Methods and intermediates for preparing compounds of the present invention include those known to one of ordinary skill in the art and as described in Lei et al., J. Org. Chem. (2005) 70:6464-6483; Su et al., Organic Letters (2005) 7:2751-2754; and Lo et al., J. Am. Chem. Soc. (2004) 126: 16077-16086, the contents of which are hereby incorporated herein by reference. One of ordinary skill in the art will also appreciate that the Examples and Schemes set forth below can be modified to prepare other compounds of the present invention.

Materials. Commercially available reagents were obtained from Aldrich Chemical Co. (Milwaukee, Wis.), Fluka Chemical Corp. (Milwaukee, Wis.), TCI America (Portland, Oreg.), and Toronto Research Chemicals Inc. (ON, Canada) and used as received unless otherwise noted. All solvents for reactions, except for CHCl₃, were dispensed from a solvent purification system that passes solvents through packed columns (THF, CH₃CN, and CH₂Cl₂: dry neutral alumina; DMF: activated molecular sieves). Water was double distilled. Reactions were monitored by analytical thin-layer chromatography using E. Merck silica gel 60 F₂₅₄ plates. Compounds were visualized with a UV lamp (λ₄₂₅) and staining with potassium permanganate.

Purification and analysis. Flash chromatography was performed using a CombiFlash Companion system (Teledyne, ISCO, Inc.) with prepacked FLASH silica columns (Biotage, Inc.). Chiral separations were performed on an Agilent 1100 HPLC system with a Chiralcel OD column (4.6×150 mm, 5 μm). ¹H NMR spectra were recorded at 23° C. on a Varian Mercury 400 (400 MHz), a Varian Unity/Inova 500 (500 MHz), and a Varian Unity/Inova 600 (600 MHz) spectrometer. Chemical shifts (δ) are reported in parts per million (ppm) downfield from tetramethylsilane and referenced to residual protium in the NMR solvent (CDCl₃, δ=7.26). Data are reported as follows: chemical shift, multiplicity (s=singlet, d=doublet, t=triplet, m=multiplet), coupling constant (J) in Hertz (Hz), and integration. ¹³C NMR spectra were recorded at 23° C. on a Varian Mercury 400 (400 MHz) and a Varian Unity/Inova 500 (500 MHz) spectrometer. Chemical shifts (δ) are reported in parts per million (ppm) downfield from tetramethylsilane and referenced to carbon resonances in the NMR solvent (CDCl₃, δ=77.0, center line). Infrared spectra were recorded as a thin film on a Nicolet AVARTAR 370 DTGS FTIR spectrometer with internal referencing. Absorption maxima (vₘₐₓ) are reported in wavenumbers (cm⁻¹). High-resolution mass spectra (HRMS) were obtained at the mass spectrometry facility at Harvard University using a mass resolution of 10.000.

General Method for Synthesis of BUCMLD Epoxyquinol Library Compounds (Table 3). The synthesis follows reported procedures; see Lei et al., J. Org. Chem. (2005) 70:6474-6483 and Su et al., Org. Lett. (2005) 7:2751-2754. The urazole-containing epoxyquinol scaffold is formed via a [4+2]cycloaddition, followed by hydrogenation of the resulting olefin. After removal of the silyl protecting group, condensation with the appropriate amine and treatment with a polymer-supported methylisatoic anhydride resin (Coppola, Tetrahedron Lett. (1998) 39:8233-8236) leads to the imine products.

TABLE 3
Exemplary Compounds of the Invention
BUCMLD-XL-184

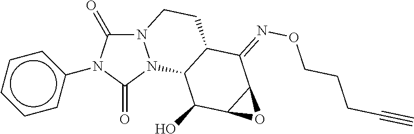

BUCMLD-B13A1

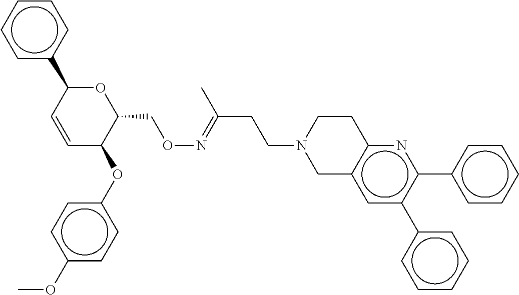

BUCMLD-XL-190

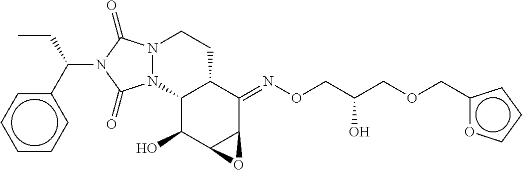

BUCMLD-B10A1

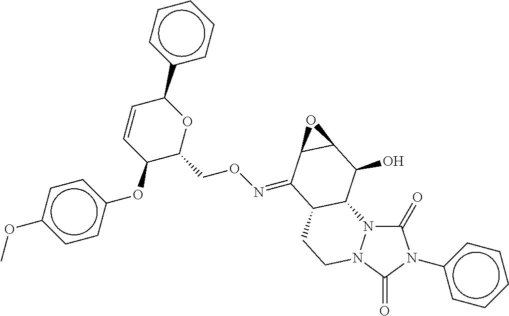

BUCMLD-XL-189

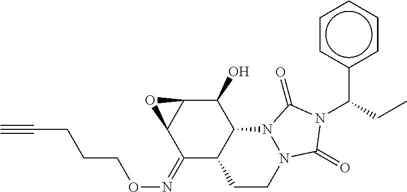

BUCMLD-B10A3

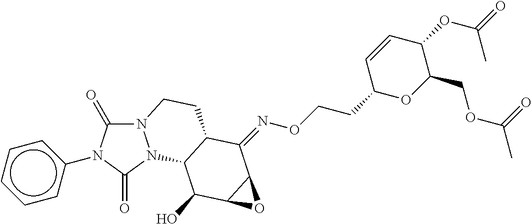

BUCMLD-B10A5

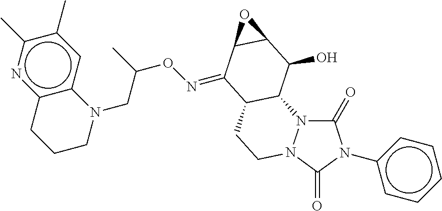

BUCMLD-B10A7

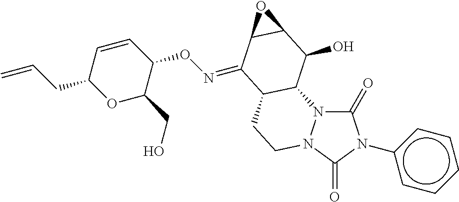

BUCMLD-B10A8

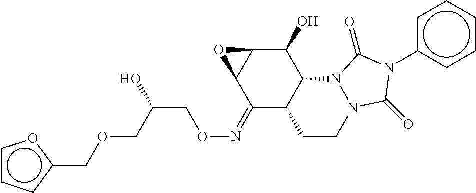

BUCMLD-B10A10

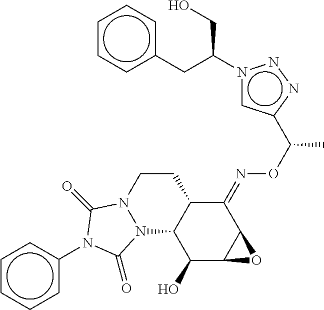

BUCMLD-B10A11

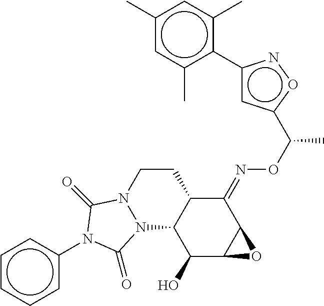

BUCMLD-B10A13

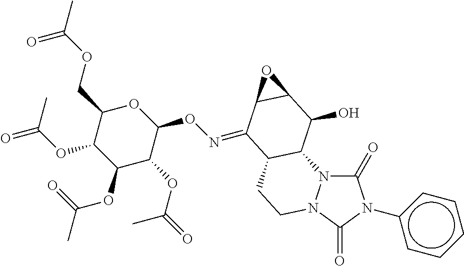

BUCMLD-B10A14

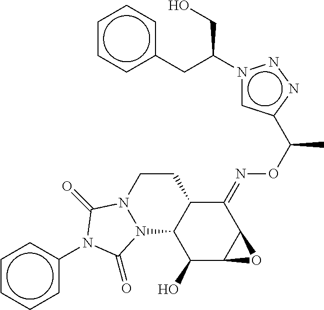

General methods and characterization for the synthesis of spiroepoxybicyclo[2.2.2]octenone derivatives (Table 4). To a suspension of alcohol (1.0 equiv) and dienophile (5.0 equiv) in solvent (CHCl₃, THF, or mixture of both, 4 ml/mmol) containing benzyltriethyl ammonium chloride (BTEAC) (0.2 equiv) was added dropwise a solution of NaIO₄ (1.1 equiv) in H₂O (1.4 ml/mmol). The reaction mixture was stirred in the dark at 23° C. for a period of time (overnight to 2 days) after which small amount of H₂O was added. The organic phase was separated, and the aqueous layer was extracted with CH₂Cl₂. The combined organics were washed with brine, dried (Na₂SO₄), and concentrated in vacuo. The crude product was purified by flash column chromatography.

TABLE 4
Exemplary Compounds of the Invention

| | |
|---|---|
| 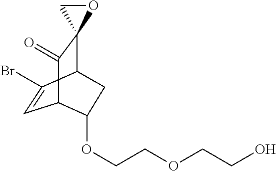 | 11 |
| 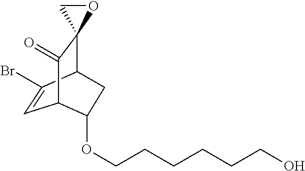 (SM_A1B2_1P32) | 12 |
| 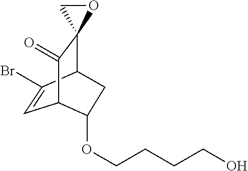 | 13 |
| 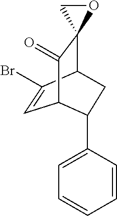 (SM_A1B5_2P24) | 14a |
| 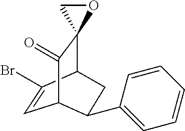 | 14b |
| 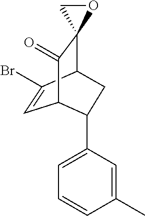 | 15 |
| 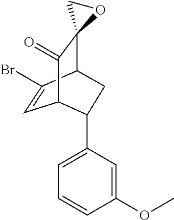 | 16 |
| 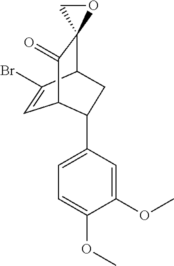 | 17 |
| 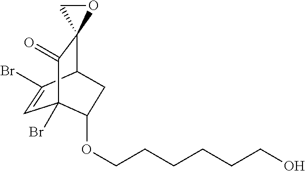 | 18 |
| 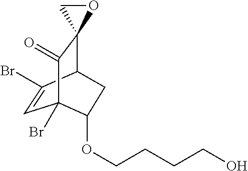 | 19 |
| 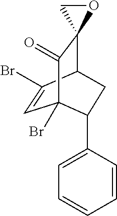 | 20 |
| 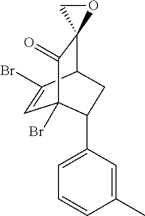 | 21 |
| 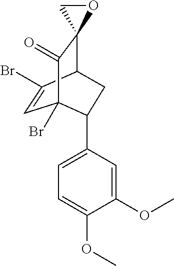 | 22 |
| 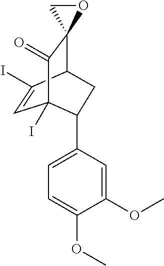 | 25 |
| 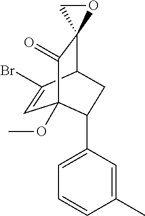 (SM_A4B6_2P123) | 26 |
| 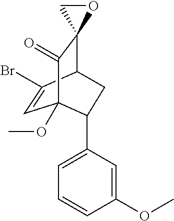 | 27 |
| 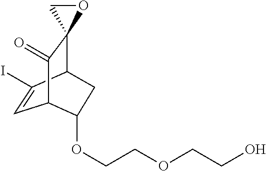 | 29 |
| 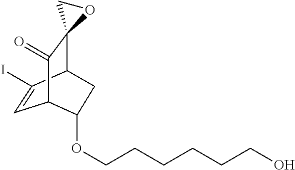 (SM_A5B2_2P142) | 30 |
| 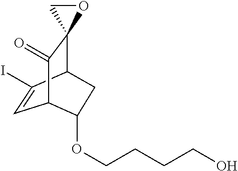 (SM_A5B3_2P141) | 31 |
| 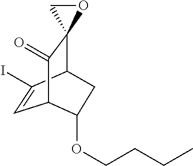 | 32 |
| 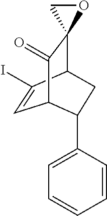 (SM_A5B5_2P118) | 33 |
| 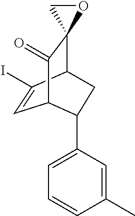 | 34 |
| 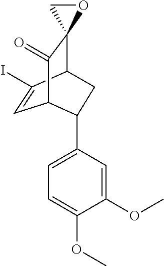 | 35 |
| 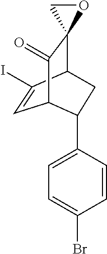 | 36 |
| 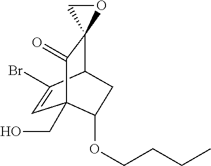 | 37 |
| 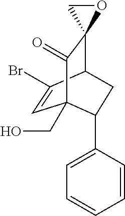 (SM_A6B5_2P100) | 38 |
| 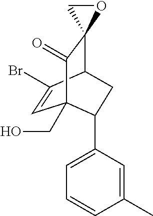 | 39 |
| 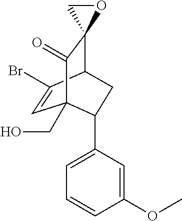 | 40 |
| 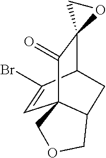 | 54a |
| 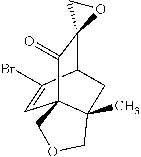 (SM_A7C2_2P155) | 55a |
| 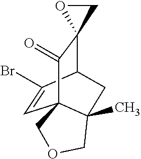 | 55b |
| 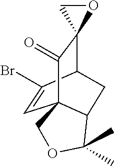 | 56a |
| 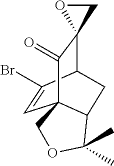 | 56b |
| 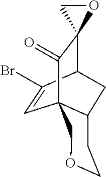 | 57a |
| 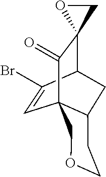 | 57b |
| 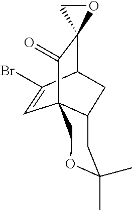 | 58a |
| 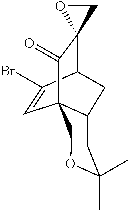 | 58b |
| 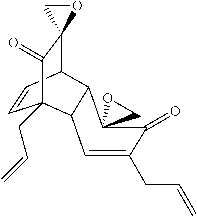 | 62 |
| 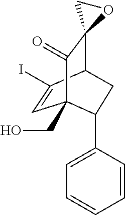 (SM_A14B5) | 67 |
| 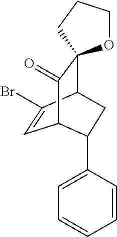 | 71 |
| 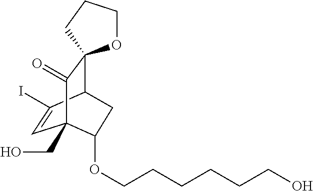 | 72 |
| 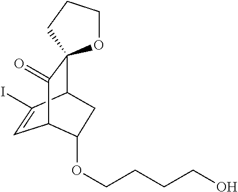 (SM_A12B3) | 73 |
| 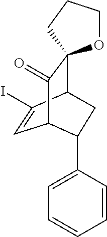 | 74 |
| 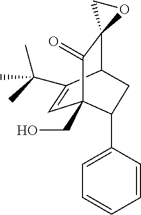 | 75 |
| 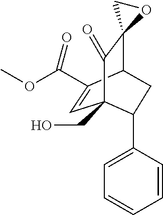 | 76 |
| 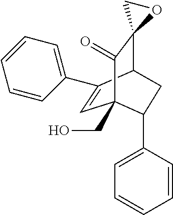 | 77 |
| 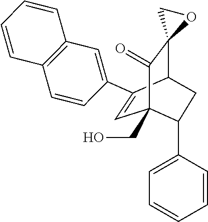 | 78 |
| 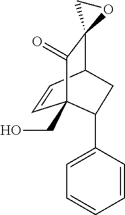 | 79 |

### Synthesis of Compound (11)

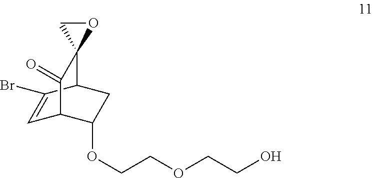

The representative procedure for the Becker-Adler/Diels-Alder reaction was followed with 5-bromo-2-hydroxybenzyl alcohol (150 mg, 0.74 mmol) and di(ethylene glycol) vinyl ether (504 μl, 3.69 mmol). The reaction was run in CHCl₃ overnight. The diastereoselectivity ratio determined by ¹H NMR analysis was greater than 95%. The crude oil was purified by flash column chromatography (10:1-1:1 hexane: EtOAc) to obtain major Diels-Alder product (11) as yellow oil (112 mg, 45%). Only the major product was isolated and characterized. ¹H NMR (400 MHz, CDCl₃): δ 6.29 (d, J=2.20 Hz, 1H), 4.00-4.19 (m, 2H), 3.70-3.85 (m, 3H), 3.54-3.66 (m, 5H), 3.19 (d, J=5.86 Hz, 1H), 3.06 (d, J=6.22 Hz, 1H), 2.72 (dd, J=5.13, 2.56 Hz, 1H), 2.54 (ddd, J=13.82, 7.96, 2.75 Hz, 1H), 1.94 (ddd, J=13.55, 2.93 Hz, 1H). ¹³C NMR (125 MHz, CDCl₃): δ 202.7, 125.4, 123.7, 75.8, 72.8, 70.5, 68.8, 62.0, 57.5, 56.0, 53.0, 48.3, 32.2.

### Synthesis of compound (12)

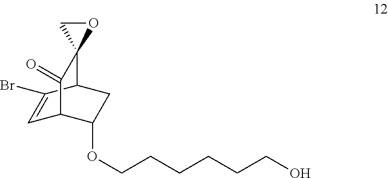

The representative procedure for the Becker-Adler/Diels-Alder reaction was followed with 5-bromo-2-hydroxybenzyl alcohol (203 mg, 1.0 mmol) and 1,6-hexanediol vinyl ether (627 μl, 5.0 mmol). The reaction was run in CHCl₃ (4 ml) overnight. The diastereoselectivity ratio determined by ¹H NMR analysis was (14:1). The crude oil was purified by flash column chromatography (4:1-1:1 hexane:EtOAc) to obtain major Diels-Alder product (12) as yellow oil (267.6 mg, 77%). Only the major product was isolated and characterized. ¹H NMR (400 MHz, CDCl₃): δ 6.30 (dd, J=6.59, 2.20 Hz, 1H), 4.03 (ddd, J=8.06, 2.93, 2.93 Hz, 1H), 3.75 (dd, J=6.59, 2.93 Hz, 1H), 3.65 (t, J=6.41 Hz, 1H), 3.45 (ddd, J=9.15, 6.59, 6.59 Hz, 1H), 3.37 (ddd, J=9.15, 6.59, 6.59 Hz, 1H), 3.16 (d, J=6.22 Hz, 1H), 3.03 (d, J=6.22 Hz, 1H), 2.68 (dd, J=5.49, 2.56 Hz, 1H), 2.49 (ddd, J=13.91, 8.24, 2.75 Hz, 1H), 1.80 (ddd, J=14.01, 3.07, 3.07 Hz, 1H), 1.51-1.62 (m, 4H), 1.32-1.42 (m, 4H). ¹³C NMR (100 MHz, CDCl₃): δ 202.8, 125.5, 123.4, 75.3, 69.3, 63.3, 57.5, 56.0, 52.9, 48.3, 32.9, 32.2, 29.8, 26.1, 25.7. IR (thin film): 3453 (w), 2926 (s), 2856 (m), 2357 (w), 1741 (m), 1112 (m), 1091 (m), 1014 (m) cm⁻¹. HRMS (ES): Calculated for C₁₅H₂₁O₄Br [M+H]: 345.0701, found: 345.0684.

### Synthesis of Compound (13)

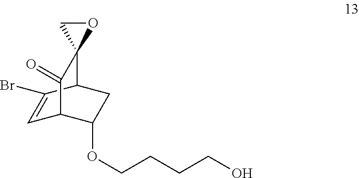

The representative procedure for the Becker-Adler/Diels-Alder reaction was followed with 5-bromo-2-hydroxybenzyl alcohol (203 mg, 1.0 mmol) and 1,4-butanediol vinyl ether (618 μl, 5.0 mmol). The reaction was run in CHCl₃ (4 ml) overnight. The diastereoselectivity ratio determined by ¹H NMR analysis was greater than 95%. The crude oil was purified by flash column chromatography (10:1-1:1 hexane:EtOAc) to obtain major Diels-Alder product (13) as oil (150 mg, 47%). Only the major product was isolated and characterized. ¹H NMR (400 MHz, CDCl₃): δ 6.31 (dd, J=6.59, 1.46 Hz, 1H), 4.06 (dddd, J=8.42, 2.93, 2.93, 1.20 Hz, 1H), 3.76 (dd, J=6.59, 2.93 Hz, 1H), 3.64 (t, J=5.86 Hz, 2H), 3.51 (ddd, J=9.15, 5.86, 5.86 Hz, 1H), 3.43 (ddd, J=9.34, 5.86, 5.86 Hz, 1H), 3.16 (d, J=5.86 Hz, 1H), 3.03 (d, J=6.22 Hz, 1H), 2.68 (dd, J=5.49, 2.56 Hz, 1H), 2.50 (ddd, J=14.10, 8.24, 2.56 Hz, 1H), 1.81 (ddd, J=13.91, 2.93, 2.93 Hz, 1H), 1.58-1.71 (m, 4H). ¹³C NMR (100 MHz, CDCl₃): δ 202.6, 125.4, 123.6, 75.5, 69.3, 62.8, 57.5, 55.9, 52.9, 48.3, 32.1, 30.1, 26.7. HRMS (ES⁺): Calculated for C₁₃H₁₇O₄Br [M+H]: 317.0388, found: 317.0388.

### Synthesis of Compound (14a and 14b)

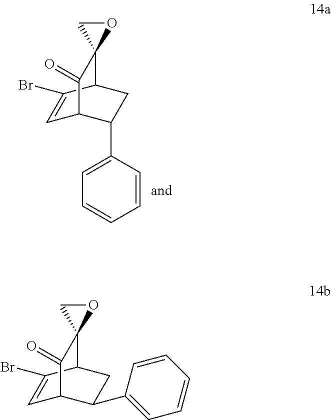

The representative procedure for the Becker-Adler/Diels-Alder reaction was followed with 5-bromo-2-hydroxybenzyl alcohol (150 mg, 0.74 mmol) and styrene (423 μl, 3.70 mmol). The reaction was run in CHCl₃ (3 ml) overnight. The diastereoselectivity ratio determined by ¹H NMR analysis was 7:1. The yellow crude oil was purified by flash column chromatography (1:1 hexane:CH₂Cl₂) to obtain major Diels-Alder product 14a as white solid (147 mg, 65%) and minor Diels-Alder product 14b as oil (21 mg, 9%). A small sample of the 14a (˜10 mg) was recrystallized in CHCl₃/hexane to give X-ray quality crystals. 14a. ¹H NMR (400 MHz, CDCl₃): δ 7.15-7.26 (m, 5H), 6.34 (dd, J=6.96, 2.20 Hz, 1H), 3.50 (ddd, J=9.89, 5.49, 1.83 Hz, 1H), 3.45 (dd, J=6.77, 2.01 Hz, 1H), 3.24 (d, J=5.86 Hz, 1H), 3.08 (d, J=5.86 Hz, 1H), 2.86 (dd, J=5.49, 2.93 Hz, 1H), 2.67 (ddd, J=13.55, 10.07, 3.11 Hz, 1H), 2.10 (ddd, J=13.64, 5.77, 2.56 Hz, 1H). ¹³C NMR (100 MHz, CDCl₃): δ 202.9, 142.8, 129.0, 127.9, 127.5, 126.8, 124.4, 57.7, 57.2, 52.8, 50.0, 41.4, 31.2. Satisfactory MS data could not be obtained. 14b. ¹H NMR (400 MHz, CDCl₃): δ 7.27-7.34 (m, 2H), 7.17-7.25 (m, 3H), 6.56 (dd, J=6.96, 2.56 Hz, 1H), 3.39 (ddd, J=11.35, 6.04, 2.75 Hz, 1H), overlap 3.31 (d, J=6.22 Hz, 1H), 3.15 (d, J=6.22 Hz, 1H), 2.86 (dd, J=5.49, 2.93 Hz, 1H), 2.49 (ddd, J=14.01, 11.26, 3.30 Hz, 1H), 2.31 (ddd, J=13.82, 6.13, 2.38 Hz, 1H).

### Synthesis of Compound (15)

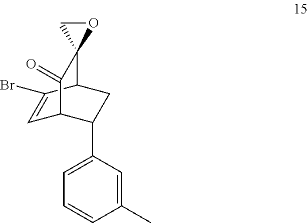

The representative procedure for the Becker-Adler/Diels-Alder reaction was followed with 5-bromo-2-hydroxybenzyl alcohol (103 mg, 0.51 mmol) and 3-methyl styrene (332 μl, 2.54 mmol). The reaction was run in CHCl₃ (2 ml) overnight. The diastereoselectivity ratio determined by ¹H NMR analysis was 11:1. The yellow crude oil was purified by flash column chromatography (100:1-10:1 hexane:EtOAc) to obtain mixture of diastereomers (white crystals, 88 mg, 54%). Only major product (15) was further characterized. ¹H NMR (400 MHz, CDCl₃): δ 7.22 (m, 1H), 6.94-7.10 (m, 3H), 6.34 (dd, J=7.14, 2.38 Hz, 1H), 3.41-3.50 (m, 2H), 3.24 (d, J=5.86 Hz, 1H), 3.08 (d, J=5.86 Hz, 1H), 2.85 (dd, J=5.49, 2.56 Hz, 1H), 2.65 (ddd, J=13.36, 10.07, 2.93 Hz, 1H), 2.35 (s, 3H), 2.10 (ddd, J=13.64, 5.77, 2.56 Hz, 1H). ¹³C NMR (100 MHz, CDCl₃): δ 202.9, 142.8, 138.7, 129.0, 128.9, 128.8, 128.1, 126.9, 124.7, 124.3, 57.7, 57.2, 52.7, 50.0, 41.3, 31.2, 21.7.

### Synthesis of Compound (16)

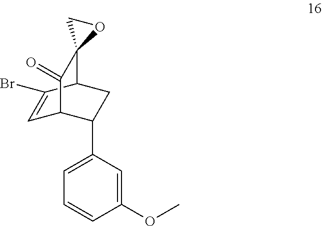

The representative procedure for the Becker-Adler/Diels-Alder reaction was followed with 5-bromo-2-hydroxybenzyl alcohol (103 mg, 0.51 mmol) and vinyl anisole (341 μl, 2.54 mmol). The reaction was run in CHCl₃ (2 ml) overnight. The diastereoselectivity ratio determined by ¹H NMR analysis was 7:1. The yellow crude oil was purified by flash column chromatography (100:1-10:1 hexane:EtOAc) to obtain mixture of diastereomers (white crystals, 160 mg, 94%). Only major product 16 was further characterized. ¹H NMR (400 MHz, CDCl₃): δ 7.05-7.12 (m, 2H), 6.83-6.89 (m, 2H), 6.33 (dd, J=6.77, 2.38 Hz, 1H), 3.79 (s, 3H), 3.45 (ddd, J=9.89, 5.68, 1.65 Hz, 1H), 3.40 (dd, J=6.77, 2.01 Hz, 1H), 3.23 (d, J=5.86 Hz, 1H), 3.07 (d, J=6.22 Hz, 1H), 2.84 (dd, J=5.13, 2.56 Hz, 1H), 2.65 (ddd, J=13.64, 10.16, 2.93 Hz, 1H), 2.05 (ddd, J=13.64, 5.58, 2.75 Hz, 1H). ¹³C NMR (100 MHz, CDCl₃): δ 203.0, 159.0, 134.8, 129.1, 128.85, 126.9, 124.3, 114.5, 114.3, 58.11, 57.2, 55.5, 52.8, 50.0, 40.6, 31.3.

### Synthesis of Compound (17)

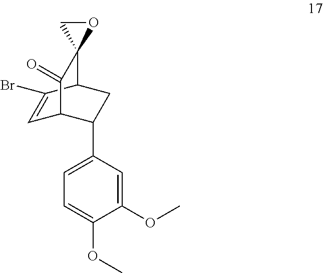

3,4-Dimethoxy styrene (Aldrich Chemical Co.) was purified by flash column chromatography prior to use. The representative procedure for the Becker-Adler/Diels-Alder reaction was followed with 5-bromo-2-hydroxybenzyl alcohol (102 mg, 0.50 mmol) and 3,4-dimethoxy styrene (370 μl, 2.50 mmol). The reaction was run in CHCl₃ (2 ml) overnight. The diastereoselectivity ratio determined by ¹H NMR analysis was 7:1. The yellow crude oil was purified by flash column chromatography (19:1-4:1 hexane:EtOAc) to obtain mixture of diastereomers (white crystals, 137 mg, 75%). Only major product (17) was further characterized. ¹H NMR (600 MHz, CDCl₃): δ 6.85 (s, 1H), 6.72-6.77 (m, 2H), 6.37 (dd, J=6.74, 2.34 Hz, 1H), 3.92 (s, 3H), 3.91 (s, 3H), 3.49 (ddd, J=9.67, 5.56, 1.46 Hz, 1H), 3.46 (dd, J=6.74, 2.05 Hz, 1H), 3.27 (d, J=5.86 Hz, 1H), 3.12 (d, J=5.86 Hz, 1H), 2.89 (dd, J=5.27, 2.64 Hz, 1H), 2.70 (ddd, J=13.47, 10.10, 2.78 Hz, 1H), 2.13 (ddd, J=13.77, 5.42, 2.78 Hz, 1H). ¹³C NMR (125 MHz, CDCl₃): δ 203.1, 149.2, 148.4, 135.3, 127.1, 124.2, 119.7, 111.4, 111.2, 58.3, 57.3, 56.2, 56.1, 52.9, 50.0, 41.1, 31.3. IR (thin film): 2937 (w), 1739 (s), 1605 (s), 1516 (s), 1464 (m), 1256 (s), 1237 (s), 1143 (s), 1026 (s), 808 (m), 765 (m), 731 (m) cm⁻¹. HRMS (ES⁺): Calculated for C₁₇H₁₇O₄Br [M+NH₄⁺]: 382.0654, found: 382.0669.

### Synthesis of Compound (18)

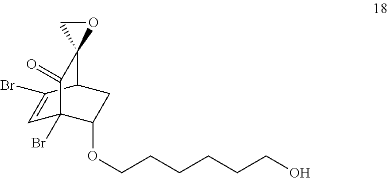

3,5-Dibromo-2-hydroxybenzyl alcohol was prepared by NaBH₄ reduction of 3,5-dibromo-2-hydroxybenzaldehyde. The representative procedure for the Becker-Adler/Diels-Alder reaction was followed with 3,5-dibromo-2-hydroxybenzyl alcohol (141 mg, 0.50 mmol) and 1,6-hexanediol vinyl ether (360 μl, 2.50 mmol) in CHCl₃ (2 ml). After an overnight run, an additional equivalent of NaIO₄ (128 mg in 840 μl water) was added. The reaction was continued to stir overnight before work-up. The diastereoselectivity ratio determined by ¹H NMR analysis was 10:1. The yellow crude oil was purified by flash column chromatography (100:1-1:1 hexane:EtOAc) to obtain mixture of diastereomers (oil, 152 mg, 72%). Only major product 18 was further characterized. ¹H NMR (600 MHz, CDCl₃): δ 6.45 (s, 1H), 3.92 (dd, J=6.88, 1.03 Hz, 1H), 3.68 (dd, J=12.01, 6.44 Hz, 2H), 3.64 (t, J=6.44 Hz, 2H), 3.28 (d, J=5.86 Hz, 1H), 3.15 (d, J=5.86 Hz, 1H), 2.74 (dd, J=4.98, 2.34 Hz, 1H), 2.60 (ddd, J=13.55, 8.13, 2.64 Hz, 1H), 2.09 (ddd, J=13.47, 2.93, 2.93 Hz, 1H), 1.57-1.69 (m, 4H), 1.39-1.48 (m, 4H). ¹³C NMR (125 MHz, CDCl₃): δ 195.2, 131.1, 121.7, 80.0, 72.7, 71.7, 63.1, 56.8, 53.6, 47.2, 33.6, 32.9, 29.9, 26.1, 25.7. HRMS (ES⁺): Calculated for C₁₅H₂₀O₄Br₂ [M+NH₄]: 440.0072, found: 440.0064.

### Synthesis of Compound (19)

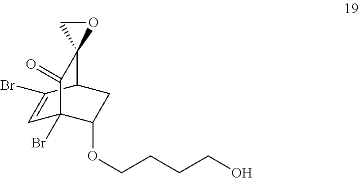

3,5-Dibromo-2-hydroxybenzyl alcohol was prepared by NaBH₄ reduction of 3,5-dibromo-2-hydroxybenzaldehyde. The representative procedure for the Becker-Adler/Diels-Alder reaction was followed with 3,5-dibromo-2-hydroxybenzyl alcohol (141 mg, 0.50 mmol) and 1,4-butanediol vinyl ether (309 μl, 2.50 mmol) in CHCl₃ (2 ml). After an overnight run, an additional equivalent of NaIO₄ (128 mg in 840 μl water) was added. The reaction was continued to stir overnight before work-up. The diastereoselectivity ratio determined by ¹H NMR analysis was 10:1. The yellow crude oil was purified by flash column chromatography (100:1-1:1 hexane:EtOAc) to obtain mixture of diastereomers (oil, 99 mg, 50%). Only major product (19) was further characterized. H NMR (600 MHz, CDCl₃): δ 6.45 (d, J=1.17 Hz, 1H), 3.95 (ddd, J=8.13, 1.21 Hz, 1H), 3.65-3.75 (m, 4H), 3.28 (d, J=5.86 Hz, 1H), 3.15 (d, J=5.86 Hz, 1H), 2.75 (dd, J=5.56, 2.93 Hz, 1H), 2.61 (ddd, J=13.62, 8.05, 2.64 Hz, 1H), 2.10 (ddd, J=13.47, 3.22, 2.34 Hz, 1H), 1.64-1.79 (m, 4H). HRMS (ES⁺): Calculated for C₁₃H₁₆O₄Br₂ [M+NH₄]: 411.9759, found: 411.9760.

### Synthesis of compound (20)

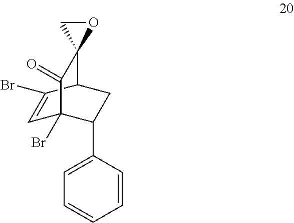

3,5-Dibromo-2-hydroxybenzyl alcohol was prepared by NaBH₄ reduction of 3,5-dibromo-2-hydroxybenzaldehyde. The representative procedure for the Becker-Adler/Diels-Alder reaction was followed with 3,5-dibromo-2-hydroxybenzyl alcohol (211 mg, 0.75 mmol) and styrene (430 μl, 3.75 mmol). The reaction was run in CHCl₃ (3 ml) overnight. The diastereoselectivity ratio determined by ¹H NMR analysis was greater than 95%. The yellow crude oil was purified by flash column chromatography (19:1-4:1 hexane: EtOAc) to obtain major Diels-Alder product (20) as white solid (216 mg, 75%). Only major product was isolated and further characterized. ¹H NMR (500 MHz, CDCl₃): δ 7.30-7.39 (m, 3H), 7.17-7.22 (m, 2H), 6.48 (d, J=2.44 Hz, 1H), 3.45 (dd, J=9.77, 5.86 Hz, 1H), 3.34 (d, J=5.86 Hz, 1H), 3.17 (d, J=5.86 Hz, 1H), 2.89 (dd, J=5.86, 2.93 Hz, 1H), 2.84 (ddd, J=13.18, 9.77, 2.93 Hz, 1H), 2.30 (ddd, J=13.55, 5.74, 2.69 Hz, 1H). ¹³C NMR (100 MHz, CDCl₃): 195.3, 141.2, 132.0, 129.4, 128.7, 128.2, 123.2, 72.2, 56.6, 53.5, 49.2, 49.1, 34.2.

### Synthesis of Compound (21)

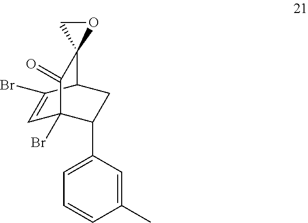

3,5-Dibromo-2-hydroxybenzyl alcohol was prepared by NaBH₄ reduction of 3,5-dibromo-2-hydroxybenzaldehyde. The representative procedure for the Becker-Adler/Diels-Alder reaction was followed with 3,5-dibromo-2-hydroxybenzyl alcohol (211 mg, 0.75 mmol) and 3-methyl styrene (491 μl, 3.75 mmol). The reaction was run in CHCl₃(3 ml) overnight. The diastereoselectivity ratio determined by ¹H NMR analysis was greater than 95%. The crude oil was purified by flash column chromatography (19:1-4:1 hexane: EtOAc) to obtain major Diels-Alder product (21) as oil that solidified upon cooling (248 mg, 83%). Only major product was isolated and further characterized. ¹H NMR (600 MHz, CDCl₃): δ 7.27-7.31 (m, 1H), 7.16-7.20 (m, 1H), 7.00-7.06 (m, 2H), 6.53 (d, J=2.34 Hz, 1H), 3.44 (dd, J=9.96, 5.56 Hz, 1H), 3.37 (d, J=5.86 Hz, 1H), 3.21 (d, J=6.15 Hz, 1H), 2.92 (dd, J=5.27, 2.64 Hz, 1H), 2.85 (ddd, J=13.40, 10.03, 3.22 Hz, 1H), 2.41 (s, 3H), 2.33 (ddd, J=13.47, 5.56, 2.64 Hz, 1H). ¹³C NMR (125 MHz, CDCl₃): δ 195.3, 141.2, 138.2, 132.1, 130.4, 128.9, 128.6, 126.3, 123.1, 72.2, 56.6, 53.5, 49.13, 49.06, 34.2, 21.8.

### Synthesis of Compound (22)

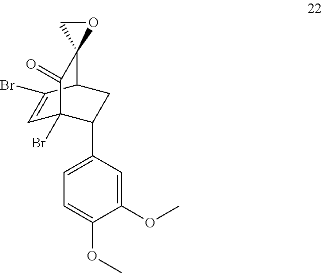

3,5-Dibromo-2-hydroxybenzyl alcohol was prepared by NaBH₄ reduction of 3,5-dibromo-2-hydroxybenzaldehyde. The commercial 3,4-dimethoxy styrene (Aldrich Chemical Co.) was repurified by flash column chromatography prior to use. The representative procedure for the Becker-Adler/Diels-Alder reaction was followed with 3,5-dibromo-2-hydroxybenzyl alcohol (141 mg, 0.50 mmol) and 3,4-dimethoxy styrene (370 μl, 2.50 mmol). The reaction was run in CHCl₃ (2 ml) overnight. The diastereoselectivity ratio determined by ¹H NMR analysis was greater than 95%. The yellow crude oil was purified by flash column chromatography (19:1-4:1 hexane:EtOAc) to obtain major Diels-Alder product (22) as white solid (158 mg, 71%). Only major product was isolated and further characterized. ¹H NMR (600 MHz, CDCl₃): δ 6.85-6.91 (m, 1H), 6.71-6.81 (m, 2H), 6.51 (d, J=2.05 Hz, 1H), 3.93 (s, 3H), 3.93 (s, 3H), 3.43 (dd, J=9.96, 5.56 Hz, 1H), 3.36 (d, J=6.15 Hz, 1H), 3.21 (d, J=5.86 Hz, 1H), 2.92 (dd, J=4.98, 2.64 Hz, 1H), 2.86 (ddd, J=13.55, 10.18, 2.93 Hz, 1H), 2.33 (ddd, J=13.62, 5.27, 2.78 Hz, 1H). ¹³C NMR (125 MHz, CDCl₃): δ 195.5, 148.9, 148.8, 133.6, 132.3, 123.0, 121.8, 112.6, 111.0, 72.8, 56.7, 56.1, 53.6, 49.0, 34.2. IR (thin film): 2936 (w), 1750 (s), 1594 (m), 1515 (s), 1463 (m), 1257 (s), 1236 (s), 1142 (s), 1024 (s), 909 (m), 803 (m), 759 (m), 725 (s) cm⁻¹. HRMS (ES⁺): Calculated for C₁₇H₁₆OBr₂ [M+N₄]: 459.9759, found: 459.9758.

### Synthesis of Compound (25)

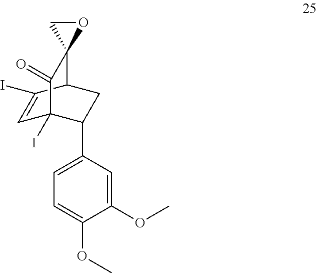

3,5-Diiodo-2-hydroxybenzyl alcohol was prepared by NaBH₄ reduction of 3,5-diiodo-2-hydroxybenzaldehyde. The commercial 3,4-dimethoxy styrene (Aldrich Chemical Co.) was repurified by flash column chromatography prior to use. The representative procedure for the Becker-Adler/Diels-Alder reaction was followed with 3,5-diiodo-2-hydroxybenzyl alcohol (94 mg, 0.25 mmol) and 3,4-dimethoxy styrene (185 μl, 1.25 mmol). The reaction was run in CHCl₃ (1 ml) overnight. The diastereoselectivity ratio determined by ¹H NMR analysis was greater than 95%. The yellow crude oil was purified by flash column chromatography (10:1-4:1 hexane:EtOAc) to obtain major Diels-Alder product (25) as white solid (115 mg, 85%). Only major product was isolated and further characterized. ¹H NMR (600 MHz, CDCl₃): δ 6.98-7.00 (m, 1H), 6.86-6.89 (m, 1H), 6.71-6.77 (m, 2H), 3.95 (s, 3H), 3.93 (s, 3H), 3.38 (dd, J=9.81, 5.56 Hz, 1H), 3.35 (d, J=6.15 Hz, 1H), 3.21 (d, J=6.15 Hz, 1H), 2.97 (dd, J=5.13, 2.64 Hz, 1H), 2.75 (ddd, J=13.40, 10.03, 2.93 Hz, 1H), 2.35 (ddd, J=13.58, 5.53, 2.71 Hz, 1H). ¹³C NMR (100 MHz, CDCl₃): δ 196.3, 149.0, 148.8, 142.9, 135.7, 110.9, 94.3, 60.2, 56.2, 56.1, 54.7, 53.6, 52.7, 50.5, 33.7.

### Synthesis of Compound (26)

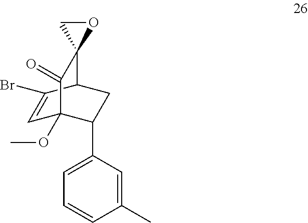

5-Bromo-3-methoxybenzyl alcohol was prepared by NaBH₄ reduction of 5-bromo-3-methoxybenzaldehyde. The representative procedure for the Becker-Adler/Diels-Alder reaction was followed with 5-Bromo-3-methoxybenzyl alcohol (75 mg, 0.32 mmol) and 3-methyl styrene (211 μl, 1.61 mmol). The reaction was run in CHCl₃ (1.35 ml) overnight. The diastereoselectivity ratio determined by ¹H NMR analysis was greater than 95%. The yellow crude oil was purified by flash column chromatography (19:1-4:1 hexane: EtOAc) to obtain major Diels-Alder product (26) as yellow solid (49 mg, 45%). Only major product was isolated and further characterized. ¹H NMR (400 MHz, CDCl₃): δ 7.17-7.23 (m, 1H), 7.09 (s, 1H), 6.97-7.01 (m, 2H), 6.49 (dd, J=2.56, 1.10 Hz, 1H), 3.37-3.43 (m, 4H), 3.25 (d, J=5.86 Hz, 1H), 3.10 (d, J=5.86 Hz, 1H), 2.80 (dd, J=5.49, 2.93 Hz, 1H), 2.72 (ddd, J=13.55, 10.25, 2.93 Hz, 1H), 2.35 (s, 3H), 2.11 (ddd, J=13.55, 5.49, 2.93 Hz, 1H). ¹³C NMR (100 MHz, CDCl₃): δ 202.7, 140.6, 138.0, 130.4, 128.8, 128.4, 128.3, 126.2, 121.5, 89.5, 57.6, 54.7, 53.0, 49.0, 45.7, 33.3, 21.8.

### Synthesis of Compound (27)

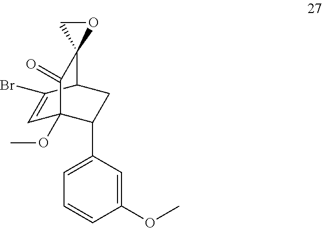

5-Bromo-3-methoxybenzyl alcohol was prepared by NaBH₄ reduction of 5-bromo-3-methoxybenzaldehyde. The representative procedure for the Becker-Adler/Diels-Alder reaction was followed with 5-Bromo-3-methoxybenzyl alcohol (75 mg, 0.32 mmol) and vinyl anisole (216 μl, 1.61 mmol). The reaction was run in CHCl₃ (1.35 ml) overnight. The diastereoselectivity ratio determined by ¹H NMR analysis was greater than 95%. The crude oil was purified by flash column chromatography (19:1-4:1 hexane:EtOAc) to obtain major Diels-Alder product (27) as white solid (88 mg, 75%). Only major product was isolated and further characterized. ¹H NMR (400 MHz, CDCl₃): δ 7.09-7.12 (m, 2H), 6.83-6.86 (m, 2H), 6.46 (dd, J=2.56, 1.10 Hz, 1H), 3.80 (s, 3H), 3.37-3.42 (m, 4H), 3.24 (d, J=6.22 Hz, 1H), 3.09 (d, J=5.86 Hz, 1H), 2.79 (dd, J=5.13, 2.56 Hz, 1H), 2.71 (ddd, J=13.64, 10.34, 2.75 Hz, 1H), 2.08 (ddd, J=13.64, 5.40, 2.93 Hz, 1H). ¹³C NMR (100 MHz, CDCl₃): δ 202.8, 159.0, 132.4, 130.3, 128.7, 121.5, 113.9, 89.6, 57.7, 55.4, 54.7, 53.0, 49.0, 45.0, 33.2.

### Synthesis of Compound (29)

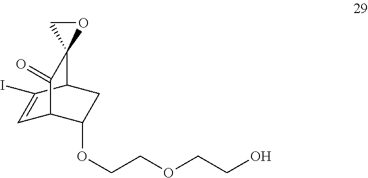

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (250 mg, 1.0 mmol) and di(ethylene glycol) vinyl ether (683 μl, 5.0 mmol). The reaction was run in the mixture of THF (400 ul) and CHCl₃ (4 ml) overnight. The diastereoselectivity ratio determined by ¹H NMR analysis was greater than 95%. The crude oil was purified by flash column chromatography (10:1-1:1 hexane:EtOAc) to obtain major Diels-Alder product (29) as oil (287 mg, 76%). Only major product was isolated and further characterized. ¹H NMR (400 MHz, CDCl₃): δ 6.64 (dd, J=6.40, 1.28 Hz, 1H), 4.12 (dddd, J=8.40, 2.92, 2.92, 0.72 Hz, 1H), 3.57-3.76 (m, 9H), 3.15 (d, J=6.22 Hz, 1H), 3.05 (d, J=6.22 Hz, 1H), 2.73 (dd, J=5.12, 2.56 Hz, 1H), 2.43 (ddd, J=14.09, 8.23, 2.93 Hz, 1H), 1.82 (ddd, J=14.00, 3.06, 3.06 Hz, 1H). ¹³C NMR (125 MHz, CDCl₃): δ 202.7, 134.1, 94.2, 75.8, 72.8, 70.5, 68.8, 62.0, 57.3, 52.8, 51.6, 31.9. HRMS (ES⁺): calculated for C₁₃H₁₇O₅I [M+H]: 381.0199, found: 381.0202.

### Synthesis of Compound (30)

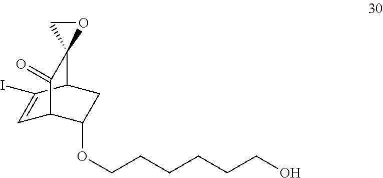

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (75 mg, 0.30 mmol) and 1,6-hexanediol vinyl ether (216 μl, 1.50 mmol) in THF (1.35 ml). After an overnight run, an additional equivalent of NaIO₄ (71 mg in 462 μl water) was added. The reaction was continued to stir overnight before work-up. The crude oil was purified by flash column chromatography (10:1-1:1 hexane:EtOAc) to obtain major Diels-Alder product (30) as oil (80 mg, 68%). Only major product was isolated and further characterized. ¹H NMR (400 MHz, CDCl₃): δ 6.61 (ddd, J=6.40, 2.20, 1.08 Hz, 1H), (dddd, J=8.06, 2.93, 1.10, 1H), 3.60-3.67 (m, 3H), 3.32-3.48 (m, 2H), 3.14 (d, J=5.86 Hz, 1H), 3.04 (d, J=6.22 Hz, 1H), 2.71 (dd, J=5.13, 2.56 Hz, 1H), 2.40 (ddd, J=14.10, 8.24, 2.56 Hz, 1H), 1.77 (ddd, J=13.91, 2.93 Hz, 1H), 1.51-1.61 (m, 4H), 1.31-1.40 (m, 4H). ¹³C NMR (100 MHz, CDCl₃): δ 202.9, 134.2, 94.0, 75.2, 69.2, 62.9, 57.3, 57.2, 52.8, 51.5, 32.8, 31.9, 29.8, 26.1, 25.7.

### Synthesis of Compound (31)

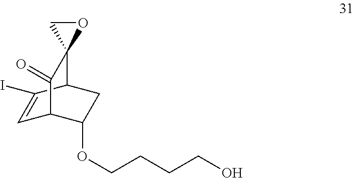

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (75 mg, 0.30 mmol) and 1,4-butanediol vinyl ether (186 μl, 1.50 mmol) in THF (1.35 ml). After an overnight run, an additional equivalent of NaIO₄ (71 mg in 462 μl water) was added. The reaction was continued to stir overnight before work-up. The crude oil was purified by flash column chromatography (10:1-1:1 hexane:EtOAc) to obtain major Diels-Alder product (31) as oil (50 mg, 46%). Only major product was isolated and further characterized. ¹H NMR (400 MHz, CDCl₃): δ 6.67 (dd, J=6.44, 1.17 Hz, 1H), 4.10 (ddd, J=8.20, 2.93, 2.93 Hz, 1H), 3.67-3.71 (m, 3H), 3.52-3.57 (m, 1H), 3.45-3.49 (m, 1H), 3.19 (d, J=6.15 Hz, 1H), 3.09 (d, J=6.15 Hz, 1H), 2.77 (dd, J=5.27, 2.34 Hz, 1H), 2.46 (ddd, J=14.06, 8.20, 2.64 Hz, 1H), 1.77 (ddd, J=14.01, 3.07, 3.07, 1H), 1.65-1.72 (m, 4H). ¹³C NMR (100 MHz, CDCl₃): δ 202.7, 134.1, 94.1, 75.4, 69.3, 57.3, 57.2, 52.9, 51.5, 31.8, 30.0, 26.6. IR (thin film): 3442 (w), 2924 (s), 2854 (m), 2360 (s), 2343 (m), 1736 (s), 1464 (w), 1357 (w) cm⁻¹. HRMS (ES⁺): Calculated for C₁₃H₁₇O₄I [M+H]: 365.0250, found: 365.0247.

### Synthesis of Compound (32)

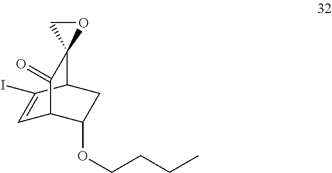

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (125 mg, 0.50 mmol) and butyl vinyl ether (323 μl, 2.50 mmol). The reaction was run in the mixture of THF (100 μl) and CHCl₃ (2 ml) overnight. The crude oil was purified by flash column chromatography (100:1-5:1 hexane:EtOAc) to obtain major Diels-Alder product 32 as oil (115 mg, 66%). Only major product was isolated and characterized. ¹H NMR (400 MHz, CDCl₃): δ 6.62 (d, J=6.22 Hz, 1H), 4.03 (dt, J=8.06, 2.93 Hz, 1H), 3.64 (dd, J=6.59, 2.20 Hz, 1H), 3.41-3.49 (m, 1H), 3.33-3.41 (m, 1H), 3.15 (d, J=6.22 Hz, 1H), 3.04 (d, J=5.86 Hz, 1H), 2.72 (dd, J=5.13, 2.56 Hz, 1H), 2.41 (ddd, J=13.91, 8.24, 1.65 Hz, 1H), 1.78 (ddd, J=14.01, 2.52 Hz, 1H), 1.47-1.57 (m, 2H), 1.28-1.40 (m, 2H), 0.91 (t, J=7.32 Hz, 3H). ¹³C NMR (100 MHz, CDCl₃): δ 202.8, 134.3, 93.9, 75.2, 69.1, 57.4, 57.3, 52.8, 51.6, 31.9, 31.9, 19.5, 14.1.

### Synthesis of Compound (33)

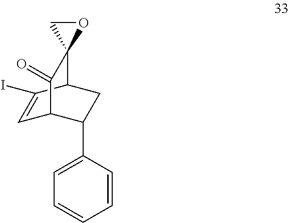

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (125 mg, 0.50 mmol) and styrene (286 μl, 2.50 mmol). The reaction was run in the mixture of THF (200 ul) and CHCl₃ (2 ml) overnight. The diastereoselectivity ratio determined by ¹H NMR analysis was 6:1. The yellow crude oil was purified by flash column chromatography (100:1-10:1 hexane: EtOAc) to obtain mixture of diastereomers (white solid, 119 mg, 68%). Only major product (33) was further characterized. ¹H NMR (500 MHz, CDCl₃): δ 7.14-7.38 (m, 5H), 6.67 (dd, J=6.84, 1.95 Hz, 1H), 3.49 (ddd, J=10.25, 5.86, 1.95 Hz, 1H), 3.35 (dd, J=6.84, 1.95 Hz, 1H), 3.24 (d, J=5.86 Hz, 1H), 3.10 (d, J=5.86 Hz, 1H), 2.91 (dd, J=4.88, 2.44 Hz, 1H), 2.59 (ddd, J=13.67, 9.77, 2.93 Hz, 1H), 2.08 (ddd, J=13.79, 5.74, 2.44 Hz, 1H). ¹³C NMR (125 MHz, CDCl₃): 202.9, 142.8, 135.7, 129.0, 127.9, 127.5, 95.3, 59.1, 57.0, 53.4, 52.7, 41.3, 30.9. IR (thin film): 2964 (w), 1737 (s), 1589 (w), 947 (m), 762 (s), 700 (s) cm⁻¹. HRMS (CI⁺): Calculated for C₁₅H₁₃O₂I [M+NH₄⁺]: 370.0317, found: 370.0304.

### Synthesis of Compound (34)

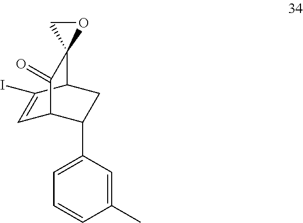

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-iodo-2-hydroxybenzyl alcohol (125 mg, 0.50 mmol) and 3-methyl styrene (327 μl, 2.50 mmol). The reaction was run in the mixture of THF (200 ul) and CHCl₃ (2 ml) overnight. The diastereoselectivity ratio determined by ¹H NMR analysis was 7:1. The yellow crude oil was purified by flash column chromatography (100:1-10:1 hexane:EtOAc) to obtain mixture of diastereomers (white solid, 95 mg, 52%). Only major product 34 was further characterized. ¹H NMR (400 MHz, CDCl₃): δ 7.18-7.24 (m, 1H), 6.94-7.11 (m, 3H), 6.66 (dd, J=6.59, 2.20 Hz, 1H), 3.45 (ddd, J=9.79, 5.77, 1.65 Hz, 1H), 3.34 (dd, J=6.59, 1.83 Hz, 1H), 3.23 (d, J=6.22 Hz, 1H), 3.09 (d, J=5.86 Hz, 1H), 2.90 (dd, J=5.13, 2.56 Hz, 1H), 2.56 (ddd, J=13.55, 10.07, 3.11 Hz, 1H), 2.35 (s, 3H), 2.07 (ddd, J=13.64, 5.77, 2.56 Hz, 1H). ¹³C NMR (100 MHz, CDCl₃): δ 202.9, 142.8, 138.6, 135.8, 128.9, 128.9, 128.2, 124.7, 95.1, 59.1, 57.0, 53.4, 52.7, 41.3, 30.9, 21.7.

### Synthesis of Compound (35)

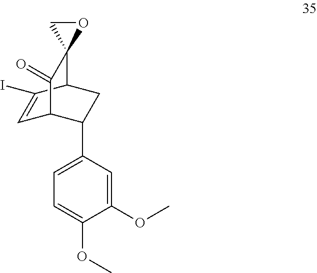

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-bromo-2-hydroxybenzyl alcohol (63 mg, 0.25 mmol) and 3,4-dimethoxy styrene (185 μl, 1.25 mmol). The reaction was run in the mixture of THF (50 ul) and CHCl₃ (1 ml) overnight. The diastereoselectivity ratio determined by ¹H NMR analysis was 5:1. The yellow crude oil was purified by flash column chromatography (100:1-5:1 hexane:EtOAc) to obtain mixture of diastereomers (white solid, 91 mg, 89%). Only major product 35 was further characterized. ¹H NMR (600 MHz, CDCl₃): δ 6.82-6.86 (m, 1H), 6.70-6.75 (m, 2H), 6.68 (dd, J=6.59, 1.90 Hz, 1H), 3.92 (s, 3H), 3.89 (s, 3H), 3.47 (ddd, J=9.67, 5.27, 1.46 Hz, 1H), 3.34 (dd, J=6.59, 1.90 Hz, 1H), 3.24 (d, J=6.15 Hz, 1H), 3.11 (d, J=6.15 Hz, 1H), 2.93 (dd, J=4.98, 2.64 Hz, 1H), 2.59 (ddd, J=13.47, 9.67, 2.93 Hz, 1H), 2.08 (ddd, J=13.77, 5.27, 2.64 Hz, 1H). ¹³C NMR (125 MHz, CDCl₃): δ 203.1, 149.2, 148.4, 136.0, 135.3, 119.8, 111.4, 111.1, 95.0, 59.6, 57.1, 56.2, 56.1, 53.3, 52.8, 41.1, 31.0. HRMS (ES⁺): Calculated for C₁₇H₁₇O₄I [M+NH₄⁺]: 430.0515, found: 430.0506.

### Synthesis of Compound (36)

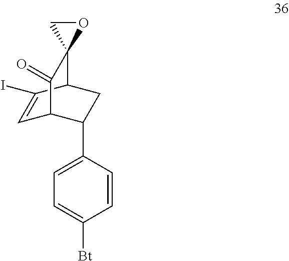

5-Iodo-2-hydroxybenzyl alcohol was prepared by NaBH₄ reduction of 5-iodo-2-hydroxybenzaldehyde. The representative procedure for the Becker-Adler/Diels-Alder reaction was followed with 5-bromo-2-hydroxybenzyl alcohol (63 mg, 0.25 mmol) and 4-bromostyrene (163 μl, 1.25 mmol). The reaction was run in the mixture of THF (100 μl) and CHCl₃ (1 ml) overnight. The diastereoselectivity ratio determined by ¹H NMR analysis was 6:1. The yellow crude oil was purified by flash column chromatography (100:1-5:1 hexane: EtOAc) to obtain mixture of diastereomers (oil, 72 mg, 70%). ¹H NMR (600 MHz, CDCl₃): δ 7.47-7.50 (m, 2H), 7.06-7.09 (m, 2H), 6.68 (dd, J=6.59, 2.20 Hz, 1H), 3.49 (ddd, J=9.77, 5.60, 1.76 Hz, 1H), 3.33 (dd, J=6.59, 1.90 Hz, 1H), 3.27 (d, J=6.15 Hz, 1H), 3.13 (d, J=6.15 Hz, 1H), 2.94 (dd, J=5.13, 2.78 Hz, 1H), 2.62 (ddd, J=13.55, 10.18, 2.93 Hz, 1H), 2.04 (ddd, J=13.80, 5.67, 2.64 Hz, 1H). ¹³C NMR (125 MHz, CDCl₃): δ 202.5, 141.8, 135.4, 132.1, 129.5, 121.4, 95.5, 58.8, 56.9, 53.3, 52.7, 40.8, 30.8.

### Synthesis of Compound (37)

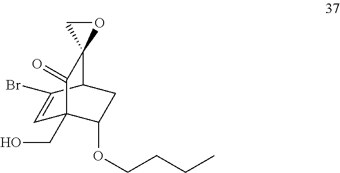

2,6-bis(hydroxymethyl)-4-bromophenol was prepared via hydroformylation of 4-bromophenol. The representative procedure for the Becker-Adler/Diels-Alder reaction was followed with 5-Bromo-2-hydroxy-1,3-phenylene)dimethanol (117 mg, 0.50 mmol) and butyl vinyl ether (323 μl, 2.50 mmol). The reaction was run in the mixture of THF (500 μl) and CHCl₃ (1.5 ml) overnight. The diastereoselectivity ratio determined by ¹H NMR analysis was 6:1. The crude oil was purified by flash column chromatography (100:1-5:1 hexane:EtOAc) to obtain major Diels-Alder product 37 as oil (107 mg, 65%). Only major product was isolated and characterized. ¹H NMR (400 MHz, CDCl₃): δ 6.28 (d, J=1.10 Hz, 1H), 3.97-4.12 (m, 3H), 3.55 (ddd, J=9.34, 6.41, 6.22 Hz, 1H), 3.36 (ddd, J=9.15, 6.59, 6.59 Hz, 1H), 3.19 (d, J=6.22 Hz, 1H), 3.05 (d, J=5.86 Hz, 1H), 2.70 (dd, J=5.13, 2.20 Hz, 1H), 2.50 (ddd, J=13.73, 7.87, 2.93 Hz, 1H), 1.91 (ddd, J=13.64, 3.07, 3.07 Hz, 1H), 1.48-1.58 (m, 2H), 1.29-1.40 (m, 2H), 0.91 (t, J=7.32 Hz, 3H). ¹³C NMR (100 MHz, CDCl₃): δ 204.6, 127.4, 123.0, 76.8, 69.8, 63.1, 61.2, 57.8, 53.1, 47.8, 32.5, 31.9, 19.5, 14.1.

### Synthesis of Compound (38)

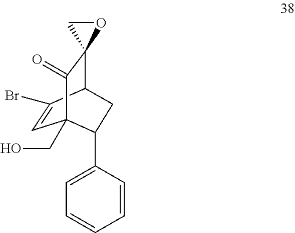

2,6-bis(hydroxymethyl)-4-bromophenol was prepared via hydroformylation of 4-bromophenol. The representative procedure for the Becker-Adler/Diels-Alder reaction was followed with 5-Bromo-2-hydroxy-1,3-phenylene)dimethanol (117 mg, 0.50 mmol) and styrene (286 μl, 2.50 mmol). The reaction was run in the mixture of THF (500 μl) and CHCl₃ (1.5 ml) overnight. The diastereoselectivity ratio determined by ¹H NMR analysis was 4:1. The crude oil was purified by flash column chromatography (100:1-5:1 hexane:EtOAc) to obtain mixture of diastereomers (yellow oil, 117 mg, 70%). Recrystallization of the mixture in CHCl₃/hexane separated out major product (38) as a clear crystal. ¹H NMR (400 MHz, CDCl₃): δ 7.24-7.35 (m, 3H), 7.15-7.21 (m, 2H), 6.23 (dd, J=2.56, 0.73 Hz, 1H), 3.59 (dd, J=12.08, 7.32 Hz, 1H), 3.43 (dd, J=12.08, 7.32 Hz, 1H), 3.37 (dd, J=9.89, 5.49 Hz, 1H), 3.26 (d, J=6.22 Hz, 1H), 3.12 (d, J=6.22 Hz, 1H), 2.86 (dd, J=5.13, 2.56 Hz, 1H), 2.77 (ddd, J=13.55, 10.25, 2.93 Hz, 1H), 2.15 (ddd, J=13.64, 5.77, 2.56 Hz, 1H).

### Synthesis of Compound (39)

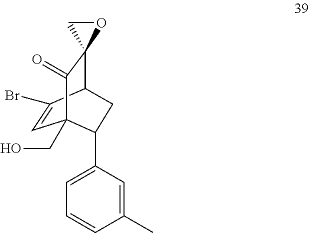

2,6-bis(hydroxymethyl)-4-bromophenol was prepared via hydroformylation of 4-bromophenol. The representative procedure for the Becker-Adler/Diels-Alder reaction was followed with 5-Bromo-2-hydroxy-1,3-phenylene)dimethanol (117 mg, 0.50 mmol) and 3-methyl styrene (327 μl, 2.50 mmol). The reaction was run in the mixture of THF (500 μl) and CHCl₃ (1.5 ml) overnight. The diastereoselectivity ratio determined by ¹H NMR analysis was 10:1. The crude oil was purified by flash column chromatography (100:1-10:1 hexane:EtOAc) to obtain major Diels-Alder product (39) (oil, 98 mg, 55%). Only major product was isolated and characterized. ¹H NMR (500 MHz, CDCl₃): δ 7.17-7.23 (m, 1H), 7.06-7.11 (m, 1H), 6.95-7.00 (m, 2H), 6.26 (d, J=1.46 Hz, 1H), 3.63 (dd, J=12.21, 6.84 Hz, 1H), 3.41 (dd, J=12.21, 7.32 Hz, 1H), 3.31 (dd, J=10.01, 5.62 Hz, 1H), 3.26 (d, J=5.86 Hz, 1H), 3.12 (d, J=6.35 Hz, 1H), 2.85 (dd, J=5.37, 2.93 Hz, 1H), 2.75 (ddd, J=13.55, 10.38, 2.93 Hz, 1H), 2.34 (s, 3H), 2.13 (ddd, J=13.79, 5.74, 2.93 Hz, 1H). ¹³C NMR (125 MHz, CDCl₃): δ 206.1, 140.7, 138.5, 129.7, 128.9, 128.6, 128.6, 125.7, 124.3, 61.8, 61.7, 57.9, 53.1, 49.5, 33.9, 21.8.

### Synthesis of Compound (40)

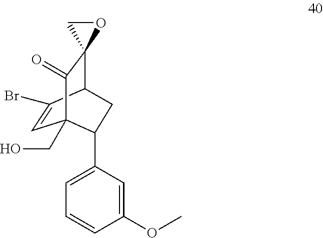

2,6-bis(hydroxymethyl)-4-bromophenol was prepared via hydroformylation of 4-bromophenol. The representative procedure for the Becker-Adler/Diels-Alder reaction was followed with 5-Bromo-2-hydroxy-1,3-phenylene)dimethanol (117 mg, 0.50 mmol) and vinyl anisole (337 μl, 2.50 mmol). The reaction was run in the mixture of THF (500 μl) and CHCl₃ (1.5 ml) overnight. The diastereoselectivity ratio determined by ¹H NMR analysis was 5:1. The crude oil was purified by flash column chromatography (100:1-4:1 hexane: EtOAc) to obtain Diels-Alder product (40) as mixture of diastereomers (oil, 142 mg, 78%). Only the major product was further characterized. ¹H NMR (500 MHz, CDCl₃): δ 7.07-7.12 (m, 2H), 6.83-6.87 (m, 2H), 6.20 (d, J=1.95 Hz, 1H), 3.79 (s, 3H), 3.58 (dd, J=12.21, 7.32 Hz, 1H), 3.45 (dd, J=12.21, 7.32 Hz, 1H), 3.34 (dd, J=10.25, 5.86 Hz, 1H), 3.25 (d, J=5.86 Hz, 1H), 3.11 (d, J=6.35 Hz, 1H), 2.84 (dd, J=5.37, 2.44 Hz, 1H), 2.75 (ddd, J=13.67, 9.77, 2.93 Hz, 1H), 2.10 (ddd, J=13.79, 5.74, 2.44 Hz, 1H). ¹³C NMR (125 MHz, CDCl₃): δ 159.2, 132.6, 129.8, 128.5, 124.4, 114.3, 62.0, 61.7, 57.9, 55.5, 53.1, 49.5, 41.9, 33.8.

### Methods and Characterization for the Synthesis of Dienophile-Tethered Alcohol Substrates.

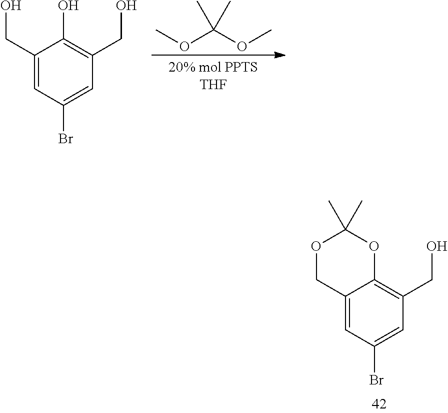

Synthesis of 6-bromo-2,2-dimethyl-4H-benzo[α][1,3]dioxin-8-yl)methanol (42). To a solution of 2,6-bis(hydroxymethyl)-4-bromophenol (3.5 g, 15 mmol) and pyridinium p-toluene sulfonate (754 mg, 3 mmol) in dry THF (150 ml) was added dimethoxy propane (18.44 ml, 150 mmol). The reaction was run under argon at room temperature overnight. The reaction mixture was diluted with THF (100 ml), and 1N HCl (150 ml) was added. After stirring for one hour, the mixture was extracted by dichloromethane, washed with brine, dried (NaSO₄), and concentrated in vacuo. Purification by flash chromatography (20:1-1:1 hexane:EtOAc) afforded the acetonide X as white crystal (3.2 g, 78%). ¹H NMR (500 MHz, CDCl₃): δ 7.32-7.34 (m, 1H), 7.04-7.06 (m, 1H), 4.81 (s, 2H), 4.62 (s, 2H), 1.54 (s, 6H). ¹³C NMR (125 MHz, CDCl₃): δ 148.2, 131.2, 129.9, 126.8, 121.3, 112.7, 100.5, 60.7, 60.6, 25.0. IR (thin film): 3404 (br, m), 2922 (s), 2853 (s), 2361 (m), 2342 (m), 1460 (s), 1375 (s), 1351 (m), 1278 (s), 1245 (s), 1202 (m), 1137 (s), 1063 (m), 957 (m), 888 (m), 840 (m), 748 (w). HRMS (ES⁺): Calculated for C₁₁H₁₃O₃Br [M+NH₄⁺]: 290.0392, found: 290.0398.

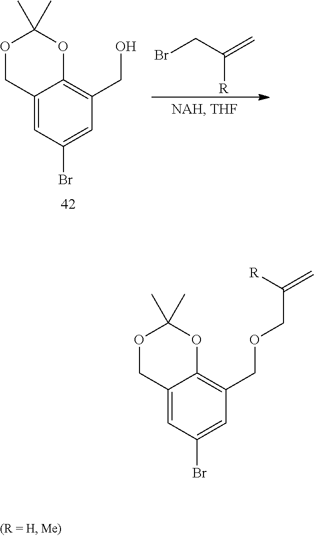

General procedure for the alkylation of acetonide 42 with bromo alkene: In an oven dried flask, NaH (55% in mineral oil, 1.21 mmol, 2.2 equiv) was rinsed with dry hexane (4 ml) and resuspended in dry THF under argon. A solution of acetonide 42 (0.55 mmol, 1.0 equiv) in dry THF (2.5 ml) was added dropwise, and the mixture was allowed to stir at 23° C. for 1 h. Bromo alkene (1.21 mmol, 2.2 equiv) was added and the reaction was continued to stir until completion. The reaction was cooled to 0° C. and quenched by the careful addition of H₂O. The resulting mixture was partitioned between Et₂O and water. The aqueous layer was extracted with Et₂O. The combined organics were washed with brine, NaHCO₃, dried (Na₂SO₄), and concentrated in vacuo.

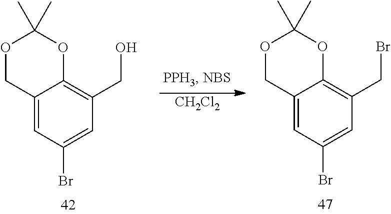

Synthesis of 6-bromo-8-(bromomethyl)-2,2-dimethyl-4H-benzo[α][1,3]dioxine (47). A solution of acetonide 42 (200 mg, 0.73 mmol) and PPh₃ (213 mg, 0.81 mmol) in dry CH₂Cl₂ (3.22 ml) was cooled to 0° C. and kept in the dark. To the reaction mixture was added NBS (146 mg, 0.82 mmol) in small portions over 1 h. The cooling bath was removed and the mixture was allowed to run at room temperature for 48 h. The solvent was evaporated under reduced pressure. Purification by flash chromatography (20:1 hexane:EtOAc) afforded the alkyl bromide product 47 as white crystal (135 mg, 55%). ¹H NMR (500 MHz, CDCl₃): δ 7.33-7.34 (m, 1H), 7.05-7.07 (m, 1H), 4.80 (s, 1H), 4.42 (s, 1H), 1.57 (s, 6H). ¹³C NMR (125 MHz, CDCl₃): δ 148.7, 312.0, 128.1, 128.0, 121.9, 112.2, 100.7, 60.5, 26.9, 24.9. IR (thin film): 2994 (m), 2924 (m), 2855 (m), 1463 (s), 1385 (s), 1375 (s), 1253 (s), 1206 (s), 1136 (s), 1066 (m), 956 (m), 893 (m), 864 (w), 838 (s), 748 (m). HRMS (EI⁺) Calculated for C₁₁H₁₂O₂Br₂ [M+H]: 333.9204, found: 333.9202.

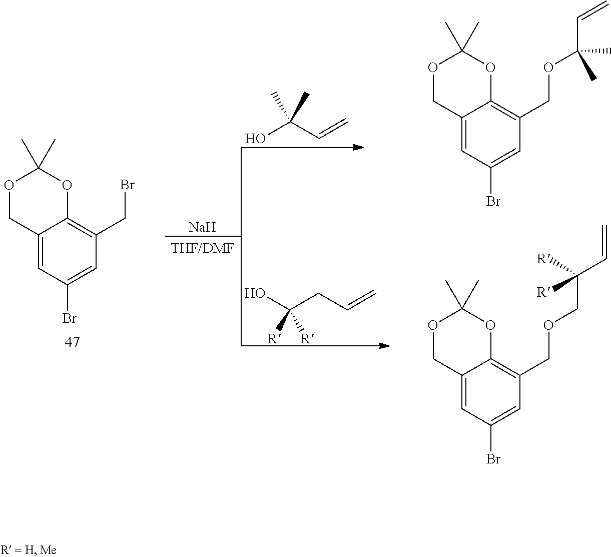

General procedure for the alkylation of alkyl bromide 47 with allyl or homo allyl alcohol: In an oven dried flask, NaH (55% in mineral oil, 0.33 mmol, 2.2 equiv) was rinsed with dry hexane (500 μl) under argon. A solution of dienophile (0.49 mmol, 3.3 equiv) in THF (150 μl) was added dropwise over 10 min. The mixture was allowed to stir at 65° C. for 2 h. A solution of 47 (0.15 mmol, 1.0 equiv) in DMF (300 μl) was added dropwise over 5 min, and the mixture was allowed to stir at 65° C. for 24-48 h. The reaction was cooled to 0° C. and quenched by the careful addition of NH₄Cl. The resulting mixture was partitioned between Et₂O and water. The aqueous layer was extracted with Et₂O. The combined organics were washed with brine, NaHCO₃, dried over Na₂SO₄, and concentrated in vacuo.

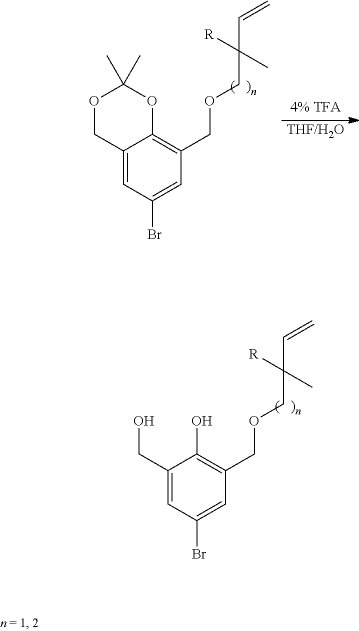

General procedure for the acetonide cleavage: To a solution of acetonide (0.3 mmol, 1 equiv) in THF/H₂O (4:1) was added trifluoroacetic acid (4 vol %) at 0° C. The reaction was slowly warmed up to 70° C. and allowed to run for 4 h. The reaction was cooled to 0° C. and quenched by the careful addition of NH₄OH. The solvent was evaporated under reduced pressure, and the remaining oil was partitioned between Et₂O and water. The aqueous layer was extracted with Et₂O. The combined organics were washed with brine, dried (Na₂SO₄), and concentrated in vacuo.

Characterization of Compound (53).

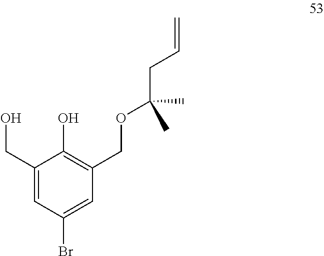

Purified by flash chromatography (10:1-1:1 hexane:EtOAc). ¹H NMR (500 MHz, CDCl₃): δ 7.27 (d, J=2.44 Hz, 1H), 7.09 (d, J=2.44 Hz, 1H), 5.79-5.89 (m, 1H), 5.13-5.17 (m, 1H), 5.11 (dd, J=3.42, 1.47 Hz, 1H), 4.67 (s, 2H), 4.64 (s, 2H), 2.36 (d, J=7.32 Hz, 2H), 1.30 (s, 6H). ¹³C NMR (125 MHz, CDCl₃): δ 153.8, 133.5, 130.5, 130.2, 129.6, 125.3, 119.0, 111.7, 77.4, 63.3, 61.9, 45.3, 25.3. HRMS (ES⁺): Calculated for C₁₄H₁₉O₃ Br [M−H₂O]: 297.0490, found: 297.0497.
Methods and Characterization for the Synthesis of spiroepoxytricyclo[5.2.2.0]undecenone Derivatives and spiroepoxytricyclo[6.2.2.0]dodecenone Derivatives

General procedure. To a solution of dienophile-tethered alcohol (0.25 mmol, 1.0 equiv) in CH₃CN (2.5 ml) was added dropwise a solution of NaIO₄ (1.25 mmol, 5.0 equiv) in H₂O (2.67 ml) at room temperature. The reaction was warmed up to 45° C. and allowed to stir for 48 h. Small amount of brine was added, and the aqueous layer was extracted with CH₂Cl₂. The combine organics were dried (Na₂SO₄), and concentrated in vacuo.

### Synthesis of Compound (54a)

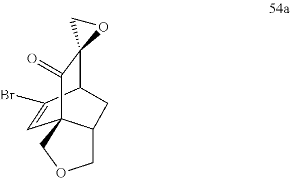

The representative procedure for the Becker-Adler/Diels-Alder reaction was followed with alcohol 45 (64 mg, 0.23 mmol), CH₃CN (2.36 ml), and NaIO₄ (251 mg, 1.17 mmol) in H₂O (2.52 ml). The diastereoselectivity ratio determined by ¹H NMR analysis was 11:1. The crude oil was purified by flash column chromatography (100:1-1:1 hexane:EtOAc) to obtain major Diels-Alder product 54a as a white solid (49 mg, 77%). A small sample of 54a (˜5 mg) was crystallized from CHCl₃/hexane to give X-ray quality crystals. ¹H NMR (600 MHz, CDCl₃): δ 6.24 (s, 1H), 4.29 (d, J=9.37 Hz, 1H), 4.14 (dd, J=7.91 Hz, 1H), 4.02 (d, J=9.37 Hz, 1H), 3.37 (dd, J=10.98, 8.05 Hz, 1H), 3.27 (d, J=5.86 Hz, 1H), 3.08 (d, J=6.15 Hz, 1H), 2.81 (ddd, J=3.75, 1.97, 1.97 Hz, 1H), 2.71-2.79 (m, 1H), 2.37 (ddd, J=12.89, 9.52, 3.66 Hz, 1H), 1.62 (ddd, J=12.89, 6.44, 6.44 Hz, 1H). ¹³C NMR (125 MHz, CDCl₃): δ 199.5, 129.7, 124.7, 71.4, 68.5, 63.9, 57.1, 52.2, 50.7, 44.0, 24.9. IR (thin film): 2937 (m), 2874 (m), 1739 (s), 1607 (m), 1385 (w), 1356 (w), 1272 (w), 1181 (m), 1055 (s), 1028 (s), 914 (m), 896 (s), 882 (m), 845 (s), 813 (w), 752 (s).

### Synthesis of Compounds (55a) and (55b)

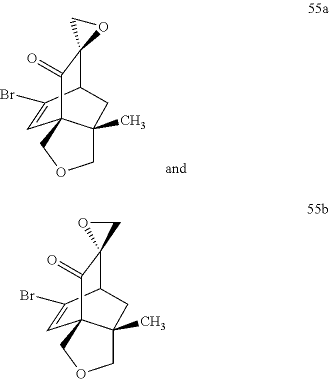

The representative procedure for the Becker-Adler/Diels-Alder reaction was followed with alcohol 46 (139 mg, 0.48 mmol), CH₃CN (4.85 ml), and NaIO₄ (516 mg, 2.41 mmol) in H₂O (5.16 ml). The diastereoselectivity ratio determined by ¹H NMR analysis was 12:1. The yellow crude oil was purified by flash column chromatography (100:1-1:1 hexane:EtOAc) to obtain major product 55a as an analytically pure oil (76 mg, 55%), and minor product 55b as a light yellow solid (6 mg, 4%). Under reduced pressure, 55a crystallized to form X-ray quality crystals. 55b was recrystallized from CHCl₃/hexane to give X-ray quality crystals. Compound (55a): ¹H NMR (600 MHz, CDCl₃): δ 6.22 (d, J=2.05 Hz, 1H), 4.35 (d, J=9.08 Hz, 1H), 3.97 (d, J=9.37 Hz, 1H), 3.76 (d, J=7.61 Hz, 1H), 3.56 (d, J=7.61 Hz, 1H), 3.31 (d, J=6.15 Hz, 1H), 3.06 (d, J=6.15 Hz, 1H), 2.77 (ddd, J=3.81, 2.34, 2.05 Hz, 1H), 1.98 (dd, J=12.89, 3.81 Hz, 1H), 1.92 (dd, J=12.89, 1.76 Hz, 1H), 1.22 (s, 3H). ¹³C NMR (125 MHz, CDCl₃): δ 198.9, 131.7, 125.5, 78.0, 67.7, 67.4, 56.6, 51.9, 50.4, 48.0, 33.4, 24.3. IR (thin film): 2935 (m), 2872 (m), 1739 (s), 1611 (m), 1451 (w), 1388 (m), 1203 (w), 1187 (m), 1143 (m), 1055 (s), 1029 (s), 960 (m), 892 (s), 868 (w), 779 (m), 750 (m), 728 (m). Compound (55b): ¹H NMR (600 MHz, CDCl₃): δ 6.23 (d, J=2.05 Hz, 1H), 4.34 (d, J=9.37 Hz, 1H), 4.01 (d, J=9.37 Hz, 1H), 3.74 (d, J=7.61 Hz, 1H), 3.55 (d, J=7.62 Hz, 1H), 3.16 (d, J=6.15 Hz, 1H), 2.94 (d, J=6.15 Hz, 1H), 2.73 (ddd, J=3.73, 2.20, 1.98 Hz, 1H), 1.99 (dd, J=13.18, 1.76 Hz, 1H), 1.87 (dd, J=13.18, 3.81 Hz, 1H), 1.20 (s, 3H).

### Synthesis of Compounds (56a) and (56b)

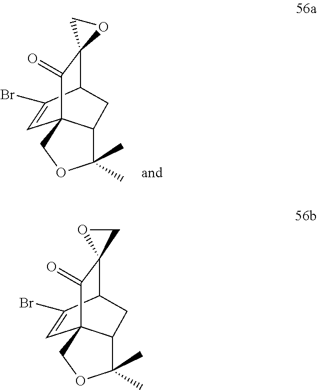

The representative procedure for the Becker-Adler/Diels-Alder reaction was followed with alcohol 49 (36 mg, 0.12 mmol), CH₃CN (1.21 ml), and NaIO₄ (128 mg, 0.6 mmol) in H₂O (1.28 ml). The diastereoselectivity ratio determined by ¹H NMR analysis was 12:1. The crude oil was purified by flash column chromatography (100:1-4:1 hexane:EtOAc) to obtain major product 56a as a white solid (15 mg, 42%), and minor product 56b as an analytically pure oil (1.07 mg, 3%). A small sample of 56a (˜5 mg) was recrystallized from CHCl₃/hexane to give X-ray quality crystals. Compound (56a): ¹H NMR (500 MHz, CDCl₃): δ 6.24 (d, J=1.46 Hz, 1H), 4.35 (d, J=9.77 Hz, 1H), 3.90 (d, J=9.77 Hz, 1H), 3.24 (d, J=5.86 Hz, 1H), 3.03 (d, J=6.35 Hz, 1H), 2.80 (ddd, J=3.91, 1.95, 1.95 Hz, 1H), 2.43-2.48 (m, 1H), 2.34 (ddd, J=12.70, 9.53, 3.91 Hz, 1H), 1.62 (ddd, J=12.70, 7.81, 1.95 Hz, 1H), 1.30 (s, 3H), 1.10 (s, 3H). Compound (56b): ¹H NMR (500 MHz, CDCl₃) δ 6.24 (dd, J=2.44, 0.98 Hz, 1H), 4.34 (d, J=9.77 Hz, 1H), 3.91 (d, J=9.77 Hz, 1H), 3.10 (d, J=6.35 Hz, 1H), 2.87 (d, J=6.35 Hz, 1H), 2.76 (ddd, J=3.78, 2.44, 2.10 Hz, 1H), 2.40 (ddd, J=9.54, 8.51, 1.00 Hz, 1H), 2.22 (ddd, J=12.94, 9.52, 3.91 Hz, 1H), 1.64 (ddd, J=12.70, 8.30, 1.95 Hz, 1H), 1.30 (s, 3H), 1.09 (s, 3H).

### Synthesis of Compounds (57a) and (57b)

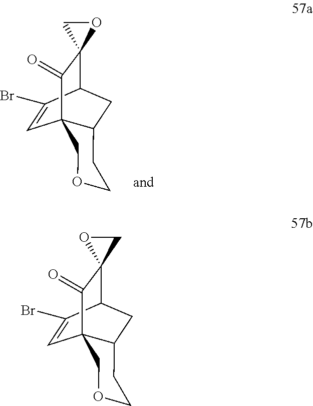

The representative procedure for the Becker-Adler/Diels-Alder reaction was followed with alcohol 52 (18 mg, 0.06 mmol), CH₃CN (627 μl), and NaIO₄ (67 mg, 0.31 mmol) in H₂O (670 μl), at 30° C. The diastereoselectivity ratio determined by ¹H NMR analysis was 10:1. The crude oil was purified by flash column chromatography (100:1-1:1 hexane:EtOAc) to obtain major product 57a as a light yellow solid (13 mg, 70%), and minor product 57b as oil (1.5 mg, 8%). A small sample of 57a (˜5 mg) was recrystallized from CHCl₃/hexane to give X-ray quality crystals. Compound (57a): ¹H NMR (400 MHz, CDCl₃): δ 6.36 (d, J=1.46 Hz, 1H), 4.10 (d, J=12.45 Hz, 1H), 3.92 (dd, J=11.72, 3.66 Hz, 1 H), 3.75 (d, J=12.45 Hz, 1H), 3.34 (ddd, J=11.99, 11.99, 2.75 Hz, 1H), 3.18 (d, J=6.22 Hz, 1H), 3.02 (d, J=6.22 Hz, 1H), 2.68 (dd, J=5.13, 2.56 Hz, 1H), 2.44 (ddd, J=12.91, 9.43, 2.93 Hz, 1H), 2.17 (ddd, J=16.84, 9.52, 4.76 Hz, 1H), 1.51-1.67 (m, 2H), 1.40 (ddd, J=13.09, 4.85, 2.56 Hz, 1H). IR (thin film): 2932 (m), 2855 (m), 1734 (s), 1604 (w), 1386 (w), 1269 (m), 1176 (m), 1137 (w), 1113 (s), 1079 (m), 959 (s), 904 (w), 858 (m), 849 (w), 835 (w), 782 (w), 748 (w), 710 (w). HRMS (CI⁺): Calculated for C₁₂H₁₃O₃Br [M+NH₄⁺]: 302.0392, found: 302.0397. Compound (57b): ¹H NMR (500 MHz, CDCl₃) δ 6.35 (dd, J=1.95, 0.98 Hz, 1H), 4.17 (d, J=12.21 Hz, 1H), 3.94 (dd, J=11.72, 4.39 Hz, 1H), 3.69 (d, J=12.21 Hz, 1H), 3.31 (ddd, J=11.96, 11.96, 2.44 Hz, 1H), 3.10 (d, J=6.35 Hz, 1H), 2.84 (d, J=5.86 Hz, 1H), 2.66 (dd, J=5.37, 2.93 Hz, 1H), 2.29 (ddd, J=13.18, 9.52, 3.17 Hz, 1H), 2.08 (ddd, J=16.60, 9.28, 4.39 Hz, 1H), 1.63-1.69 (m, 1H), 1.46-1.54 (m, 2H).

### Synthesis of Compounds (58a) and (58b)

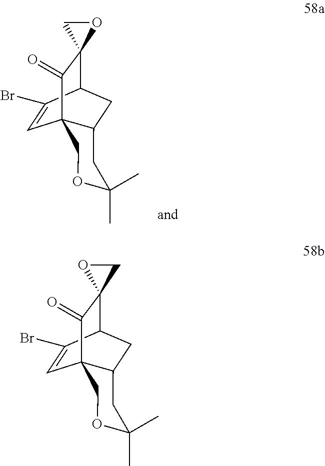

The representative procedure for the Becker-Adler/Diels-Alder reaction was followed with 53 (67 mg, 0.21 mmol), CH₃CN (2.14 ml), and NaIO₄ (227 mg, 1.06 mmol) in H₂O (2.27 ml). The diastereoselectivity ratio determined by ¹H NMR analysis was 14:1. The yellow crude oil was purified by flash column chromatography (100:1-1:1 hexane:EtOAc) to obtain major Diels-Alder product 58a as a white solid (49 mg, 74%), and minor Diels-Alder product 58b as a yellow oil (3 mg, 4%). Compound (58a): ¹H NMR (600 MHz, CDCl₃): δ 6.38 (s, 1H), 4.10 (d, J=12.89 Hz, 1H), 3.89 (d, J=12.59 Hz, 1H), 3.21 (d, J=5.86 Hz, 1H), 3.05 (d, J=6.15 Hz, 1H), 2.71 (dd, J=5.22, 2.88 Hz, 1H), 2.36-2.48 (m, 2H), 1.57 (dd, J=13.33, 4.25 Hz, 1H), 1.33-1.43 (m, 2H), 1.25 (s, 6H); ¹³C NMR (100 MHz, CDCl₃): δ 203.8, 130.0, 122.6, 72.0, 62.0, 58.2, 55.0, 52.6, 48.7, 41.8, 32.5, 31.2, 29.7, 21.3. IR (thin film): 2973 (m), 2929 (m), 1735 (s), 1605 (w), 1382 (w), 1369 (w), 1284 (w), 1256 (w), 1167 (m), 1150 (w), 1080 (m), 959 (w), 935 (w), 857 (w), 842 (w), 789 (w), 759 (w), 747 (w), 712 (w). HRMS (ES⁺): Calculated for C₁₄H₁₇O₃Br [M+NH₄⁺]: 330.0705, found: 330.0706. Compound (58b): ¹H NMR (600 MHz, CDCl₃): δ 6.37 (d, J=1.76 Hz, 1H), 4.05 (d, J=12.89 Hz, 1H), 3.96 (d, J=12.59 Hz, 1H), 3.15 (d, J=6.15 Hz, 1H), 2.87 (d, J=6.15 Hz, 1H), 2.69 (dd, J=4.98, 2.64 Hz, 1H), 2.27-2.34 (m, 2H), 1.28-1.59 (m, 3H), 1.26 (s, 3H), 1.24 (s, 3H); ¹³C NMR (100 MHz, CDCl₃): δ 204.2, 128.5, 124.1, 72.0, 62.2, 60.0, 54.9, 52.0, 48.4, 42.2, 31.6, 31.1, 30.9, 21.3.
Methods and Characterization for the Synthesis of ethenonaphthalene bis(spirooxirane)dione:

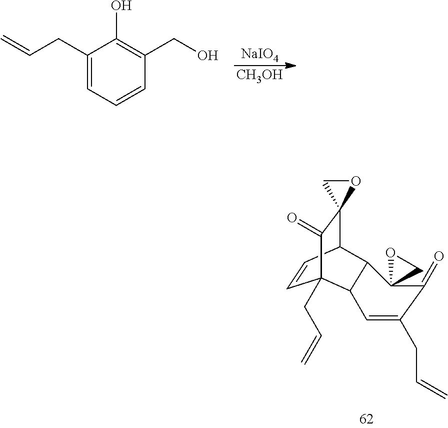

Synthesis of 1,3,4,4a,5,8a-Hexahydro-1,7-diallyl-1,4-ethenonaphthalene-3,5-bis(spirooxirane)-2,6-dione (62). 3-Allyl-2-hydroxybenzyl alcohol was obtained from NaBH₄ reduction of 3-allyl-2-hydroxybenzaldehyde. To a solution of 3-allyl-2-hydroxybenzyl alcohol (25 mg, 0.15 mmol) in methanol (507 μl) at 0° C. was added dropwise a solution of NaIO₄ (36 mg, 0.17 mmol) in H₂O (600 μl). The mixture was warmed up to 23° C. and allowed to stir for 2 h. Additional NaIO₄ (36 mg in 600 μl H₂O) was added, and the reaction was allowed to run overnight. The suspension was filtered through a pad of glass wool. The filtrate was diluted with brine (500 μl) and extracted with CH₂Cl₂ (3×1 ml). The combined organic layer was washed with brine, dried, and concentrated in vacuo to afford white solid crude. The resulting crude was redissolved in small amount of CH₂ Cl₂ and purified by flash column chromatography (100:1-1:1 hexane: EtOAc) to obtain dimer 62 as a white solid (20 mg, 80%). ¹H NMR (400 MHz, CDCl₃): δ 6.59 (dd, J=8.06, 6.59 Hz, 1H), 6.52 (d, J=4.76 Hz, 1H), 5.92-6.05 (m, 2H), 5.69-5.82 (m, 2H), 5.02-5.26 (m, 4H), 3.32 (dd, J=8.97, 4.58 Hz, 1H), 3.15 (d, J=6.22 Hz, 1H), 2.92-3.07 (m, 2H), 2.91 (d, J=5.86 Hz, 1H), 2.88 (ddd, J=6.68, 1.97 Hz, 1H), 2.82 (dd, J=10.98, 6.22 Hz, 2H), 2.69-2.78 (m, 2H), 2.50 (dd, J=14.28, 8.42 Hz, 1H). ¹³C NMR (100 MHz, CDCl₃): δ 204.7, 192.8, 141.9, 140.3, 134.6, 133.9, 133.8, 133.8, 125.1, 119.2, 117.8, 59.1, 58.8, 58.1, 54.1, 41.1, 40.2, 39.9, 34.3, 34.0. HRMS (ES⁺): Calculated for C₂₀H₂₀O₄ [M+NH₄⁺]: 342.1705, found: 342.1708.

Methods and Characterization for the Synthesis of Bicyclo[2.2.2]octenone Analogues:

### Synthesis of Compound (67)

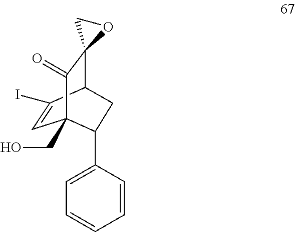

2,6-Bis(hydroxymethyl)-4-iodophenol (66) was prepared as previously described (Crisp, G. T.; Turner, P. D. Tetrahedron 2000, 56, 407-415). The representative procedure for the Becker-Adler/Diels-Alder reaction was followed with 66 (36 mg, 0.125 mmol) and styrene (75 μl, 0.65 mmol). The reaction was run in THF (0.5 ml) overnight. The diastereoselectivity ratio determined by ₁H NMR analysis was 12:1. The crude oil was purified by flash column chromatography (100:1-4:1 hexane:EtOAc) to obtain mixture of diastereomers (white solid mixed with oil, mg, %). The mixture was recrystallized in CHCl₃/hexane to give major product 67 as white crystal. The minor product was in mixture with some unknown impurities. Only major product 67 was further characterized and tested. ₁H NMR (500 MHz, CDCl₃): δ 7.28-7.38 (m, 3H), 7.21 (s, 2H), 6.59 (dd, J=2.05, 0.59 Hz, 1H), 3.61 (dd, J=12.30, 6.74 Hz, 1H), 3.45 (dd, J=12.15, 6.88 Hz, 1H), 3.40 (dd, J=10.25, 5.86 Hz, 1H), 3.29 (d, J=6.15 Hz, 1H), 3.16 (d, J=6.15 Hz, 1H), 2.95 (dd, J=5.27, 2.64 Hz, 1H), 2.72 (ddd, J=13.62, 10.25, 3.08 Hz, 1H), 2.15 (ddd, J=13.77, 5.86, 2.64 Hz, 1H). ¹³C NMR (125 MHz, CDCl₃): δ 206.2, 140.7, 137.3, 129.1, 129.0, 128.9, 127.8, 95.4, 62.9, 61.4, 57.7, 53.0, 52.9, 42.5, 33.6. IR (thin film): 3558 (br), 2940 (m), 2877 (m), 1730 (s), 1492 (m), 1457 (m), 1389 (m), 1053 (m), 999 (m), 764 (s), 704 (s). HRMS (ES+): Calculated for C₁₆H₁₅O₃I [M+NH₄₊]: 400.0410, found: 400.0405.

General procedure for preparing 4-substituted 2-(3-hydroxypropyl)-phenol from the corresponding 6-substituted 3,4-dihydrocoumarins: To a suspension of LiAlH₄ (1.60 mmol, 1.6 equiv) in ether (4.3 ml) was added dropwise a solution of 6-substituted-3,4-dihydrocoumarin (1.0 mmol, 1.0 equiv) in ether (4.3 ml) over a period of 30 min under nitrogen. The reaction was refluxed for 3 h after which excess LiAlH₄ was decomposed by wet ether (15 ml). The formed precipitate was then dissolved with diluted H₂SO₄ (10% by volume). The ethereal layer was washed with brine (2×20 ml), saturated NaHCO₃ (2×20 ml), dried (Na₂SO₄), and concentrated in vacuo to afford solid crude product. The crude product was purified by flash column chromatography.

### Synthesis of 2-(3-hydroxypropyl)-4-bromophenol (69)

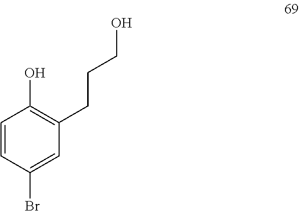

The crude product was purified by flash column chromatography (10:1-1:1 hexane:EtOAc) to obtain the alcohol product as an analytically pure oil which solidified upon standing (200 mg, 87%). ¹H NMR (500 MHz, CDCl₃): δ 7.18-7.23 (m, 2H), 6.75 (d, J=8.30 Hz, 1H), 3.66 (t, J=5.86 Hz, 2H), 2.75 (t, J=6.84 Hz, 2H), 1.85-1.91 (m, 2H). ₁₃C NMR (125 MHz, CDCl₃): δ 154.0, 133.4, 130.6, 129.9, 118.2, 112.9, 60.9, 32.1, 25.3. IR (thin film): 3261 (br), 2931 (s), 2873 (s), 1493 (s), 1416 (s), 1268 (s), 1243 (s), 1172 (s), 1036 (s), 812 (s), 628 (s). HRMS (ES+): Calculated for C₉H₁₁O₂Br [M+CH₃CN+H]: 272.0286 found: 272.0276.

### Synthesis of 2-(3-hydroxypropyl)-4-iodophenol (70)

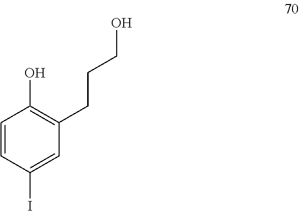

The crude product was purified by flash column chromatography (10:1-1:1 hexane:EtOAc) to obtain the alcohol product as an analytically pure oil which solidified upon standing (250 mg, 90%). ₁H NMR (500 MHz, CDCl₃): δ 7.36-7.41 (m, 2H), 6.64 (d, J=8.30 Hz, 1H), 3.65 (t, J=5.86 Hz, 2H), 2.79 (t, J=6.84 Hz, 1H), 2.72 (t, J=6.84 Hz, 2H), 1.84-1.92 (m, 2H). ¹³C NMR (125 MHz, CDCl₃): δ 155.0, 139.3, 136.6, 130.4, 118.9, 82.9, 60.8, 32.1, 25.1. IR (thin film): 3300 (br), 2943 (s), 2882 (m), 1490 (s), 1411 (s), 1268 (s), 1247 (s), 1171 (s), 1115 (s), 1035 (m), 812 (s). HRMS (ES+): Calculated for C₉H₁₁O₂I [M+NH₄₊]: 296.0148, found: 296.0146.

### Synthesis of Compound (71)

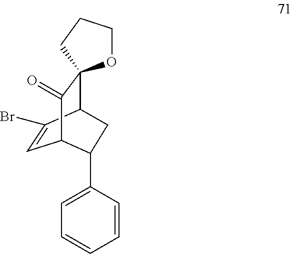

To a solution of 69 (58 mg, 0.25 mmol) and styrene (286 ul, 2.5 mmol) in dry acetonitrile (4.5 ml) was added over 10 min a solution of [bis(trifluoroacetoxy)iodo]benzene (130 mg, 0.3 mmol) in the same solvent (0.5 ml). The mixture was stirred overnight and concentrated in vacuo to afford brown oil crude product. The resulting oil was purified by flash column chromatography (20:1-5:1 hexane:EtOAc) to obtain Diels-Alder product 71 as oil (46 mg, 55%). ₁H NMR (500 MHz, CDCl₃): δ 7.21-7.33 (m, 3H), 7.14-7.18 (m, 2 H), 6.27 (dd, J=6.84, 2.44 Hz, 1H), 3.99-4.07 (m, 2H), 3.44 (dd, J=8.30, 6.84 Hz, 1H), 3.29 (dd, J=6.84, 1.46 Hz, 1H), 3.04 (dd, J=5.37, 2.93 Hz, 1H), 2.72 (ddd, J=13.31, 9.89, 3.17 Hz, 1H), 2.10-2.19 (m, 2H), 1.93-2.06 (m, 2H), 1.85 (ddd, J=13.43, 6.10, 2.44 Hz, 1H). ₁₃C NMR (125 MHz, CDCl₃): δ 143.7, 128.9, 127.9, 127.1, 126.4, 125.6, 81.6, 70.4, 57.8, 54.2, 41.2, 34.0, 30.2, 26.7. IR (thin film): 2956 (s), 2854 (m), 2359 (w), 2340 (w), 1734 (s), 1604 (w), 1456 (w), 1069 (m), 766 (m), 700 (m). HRMS (ES₊) Calculated for C₁₇H₁₇O₂Br [M+H]: 333.0490, found: 333.0491.

### Synthesis of Compound (72)

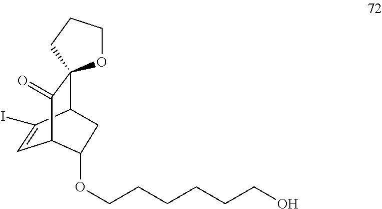

To a solution of 70 (70 mg, 0.25 mmol) in dry acetonitrile (4.5 ml) was added dropwise a solution of [bis(trifluoroacetoxy)iodo]benzene (161 mg, 0.375 mmol) in the same solvent (0.5 ml). The reaction mixture was allowed to stir for 10 min. Solid K₂CO₃ (200 mg) was added, followed (after 5 min) by 1,6-hexanediol vinyl ether (618 ul, 5.0 mmol). The mixture was stirred for 48 h, filtered, and concentrated in vacuo to afford brown oil crude product. The resulting crude oil was purified by flash column chromatography (10:1-1:1 hexane:EtOAc) to obtain Diels-Alder product 72 as oil (92 mg, 87%). ₁H NMR (500 MHz, CDCl₃): δ 6.51 (dd, J=6.59, 1.22 Hz, 1H), 3.88-3.98 (m, 3H), 3.63 (t, J=6.59 Hz, 2H), 3.50 (dd, J=6.59, 2.69 Hz, 1H), 3.31-3.44 (m, 2H), 2.92 (dd, J=4.88, 2.44 Hz, 1H), 2.48 (ddd, J=13.67, 8.30, 2.93 Hz, 1H), 2.01-2.15 (m, 2H), 1.89-2.01 (m, 2H), 1.48-1.61 (m, 5H), 1.28-1.42 (m, 4H).

### Synthesis of Compound (73)

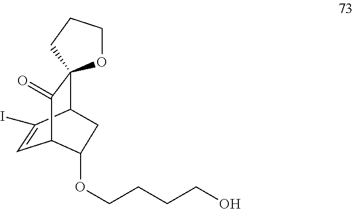

To a solution of 70 (70 mg, 0.25 mmol) in dry acetonitrile (4.5 ml) was added dropwise a solution of [Bis(trifluoroacetoxy)iodo]benzene (161 mg, 0.375 mmol) in the same solvent (0.5 ml). The reaction mixture was allowed to stir for 10 min. Solid K₂CO₃ (200 mg) was added, followed (after 5 min) by 1,4-butanediol vinyl ether (618 ul, 5.0 mmol). The mixture was stirred for 48 h, filtered, and concentrated in vacuo to afford brown oil crude product. The resulting crude oil was purified by flash column chromatography (10:1-1:1 hexane:EtOAc) to obtain Diels-Alder product 73 as oil (79 mg, 80%). ₁H NMR (500 MHz, CDCl₃): δ 6.52 (dd, J=6.35, 1.46 Hz, 1H), 3.96-4.01 (m, 1H), 3.90-3.95 (m, 2H), 3.63 (t, J=5.86 Hz, 2H), 3.51 (dd, J=6.59, 2.69 Hz, 1H), 3.37-3.49 (m, 2H), 2.94 (dd, J=4.88, 2.44 Hz, 1H), 2.50 (ddd, J=13.67, 8.30, 2.93 Hz, 1H), 2.02-2.14 (m, 2H), 1.90-2.01 (m, 2H), 1.58-1.67 (m, 4H), 1.53 (ddd, J=13.67, 3.17 Hz, 1H). ₁₃C NMR (125 MHz, CDCl₃): 133.9, 96.0, 81.7, 75.5, 70.0, 69.1, 62.8, 57.8, 55.2, 33.9, 30.3, 30.2, 26.8, 26.7. IR (thin film): 3436 (br), 2925 (m), 2868 (m), 1730 (s), 1356 (w), 1064 (s), 986 (m). HRMS (ES₊): Calculated for C₁₅H₂₁O₄I [M+H]: 393.0563, found: 393.0571.

### Synthesis of Compound (74)

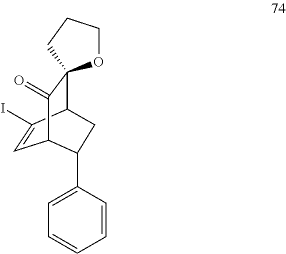

To a solution of 70 (70 mg, 0.25 mmol) in dry acetonitrile (4.5 ml) was added over 10 min a solution of [bis(trifluoroacetoxy)iodo]benzene (130 mg, 0.3 mmol) in the same solvent (0.5 ml). The reaction mixture was allowed to stir for 10 min, after which styrene (286 ul, 2.5 mmol) was added. The mixture was stirred overnight and concentrated in vacuo to afford brown oil crude product. The resulting oil was purified by flash column chromatography (20:1-5:1 hexane:EtOAc) to obtain Diels-Alder product 74 as oil which solidified at −4° C. (50 mg, 53%). Recrystallization in CHCl₃/hexane yielded white needle crystals. ₁H NMR (500 MHz, CDCl₃): δ 7.21-7.33 (m, 3H), 7.13-7.17 (m, 2H), 6.59 (dd, J=6.59, 2.20 Hz, 1H), 3.97-4.07 (m, 2H), 3.43 (dd, J=8.55, 7.08 Hz, 1H), 3.21 (dd, J=6.84, 1.46 Hz, 1H), 3.13 (dd, J=4.88, 2.44 Hz, 1H), 2.64 (ddd, J=13.31, 9.89, 3.17 Hz, 1H), 2.12-2.19 (m, 2H), 1.98-2.05 (m, 2H), 1.83 (ddd, J=13.18, 6.35, 2.44 Hz, 1H). ₁₃C NMR (125 MHz, CDCl₃): δ 143.7, 135.4, 128.9, 127.9, 127.1, 97.0, 81.9, 77.5, 77.3, 77.0, 70.2, 59.4, 57.2, 41.2, 34.1, 29.9, 26.8. IR (thin film): 2951 (m), 1732 (s), 1590 (w), 1497 (w), 1456 (w), 1067 (m), 980 (w), 766 (m), 700 (m). HRMS (ES₊): Calculated for C₁₇H₁₇O₂I [M+H]: 381.0351, found: 381.0359.

### Synthesis of Compound (75)

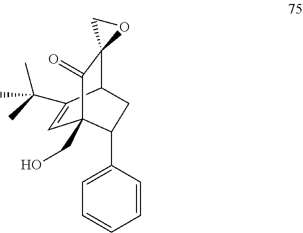

2,6-Bis(hydroxymethyl)-4-tert-butylphenol was prepared via NaBH₄ reduction of 4-tert-butyl-2,6-diformylphenol. The representative procedure for the Becker-Adler/Diels-Alder reaction was followed with 2,6-bis(hydroxymethyl)-4-tert-butylphenol (105 mg, 0.5 mmol) and styrene (573 μl, 10.0 mmol). The reaction was run in CHCl₃ (2 ml) for 48 hours. The crude oil was purified by flash column chromatography (10:1-2:1 hexane:EtOAc) to obtain the spiroepoxy hexacyclodienone intermediate as yellow oil (52 mg) and Diels-Alder product 75 as oil (60 mg, 38%). ₁H NMR (600 MHz, CDCl₃): δ 7.18-7.35 (m, 5H), 5.66 (s, 1H), 3.56 (d, J=11.57 Hz, 1H), 3.51 (d, J=11.57 Hz, 1H), 3.41 (dd, J=10.32, 6.08 Hz, 1 H), 3.26 (d, J=6.15 Hz, 1H), 2.96 (d, J=6.00 Hz, 1H), 2.78-2.86 (m, 2H), 2.51 (br. s., 1 H), 1.80-1.86 (m, 1H), 1.24 (s, 9H). ¹³C NMR (125 MHz, CDCl₃): δ 208.5, 157.1, 141.9, 128.8, 128.7, 127.4, 117.3, 62.4, 58.7, 58.4, 53.4, 42.6, 40.0, 35.4, 34.2, 28.0.

### Synthesis of Compound (76)

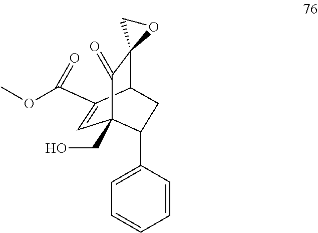

Ethyl 4-hydroxy-3,5-bis(hydroxymethyl)benzoate was prepared from ethyl 4-hydroxybenzoate as previously described (Haba, K.; Popkov, M.; Shamis, M.; Lerner, R. A.; Barbas III, C. F.; Shabat, D. Ang. Chem. Int. Ed. 2005, 44, 716-720). To a solution of 4-hydroxy-3,5-bis(hydroxymethyl)benzoate (170 mg, 0.75 mmol) and styrene (1.3 ml, 11.25 mmol) in MeOH (7.5 ml) was added a solution of NaIO₄ (241 mg, 1.125 mmol) in water (1.6 ml) over 3 hours with the aid of a syringe pump at 50° C. The reaction was stirred at that temperature for 4 hours. The volatile was removed under reduced pressure, and the remaining mixture was partitioned between water and EtOAc. The aqueous layer was extracted with EtOAc, dried over Na₂SO₄, and evaporated in vacuo. The crude product was purified by flash column chromatography (100:1-4:1 hexane:EtOAc) to obtain Diels-Alder product 76 as a white solid (75 mg, 30%). A good amount of 4-hydroxy-3,5-bis(hydroxymethyl)benzoate was also recovered. ₁H NMR (600 MHz, CDCl₃): δ 7.25-7.35 (m, 3H), 7.11-7.17 (m, 3H), 4.42 (q, J=7.18 Hz, 2H), 3.75 (dd, J=12.15, 6.00 Hz, 1H), 3.49-3.54 (m, 1H), 3.44 (dd, J=10.03, 6.22 Hz, 1H), 3.36 (dd, J=4.69, 2.64 Hz, 1H), 3.20 (d, J=6.30 Hz, 1H), 3.05 (d, J=6.30 Hz, 1H), 2.87 (ddd, J=13.58, 10.29, 2.93 Hz, 1H), 2.41 (br. s., 1H), 1.97 (ddd, J=13.62, 6.15, 2.64 Hz, 1H), 1.43 (t, J=7.10 Hz, 3H). ₁₃C NMR (125 MHz, CDCl₃): δ 206.7, 163.6, 140.9, 139.4, 138.5, 129.0, 128.7, 127.8, 61.7, 61.5, 60.7, 57.6, 53.5, 43.1, 38.3, 33.8, 14.5.

### Synthesis of Compound (77)

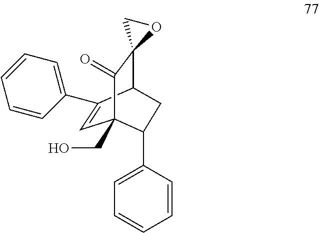

The preparation of 2,6-bis(hydroxymethyl)-4-phenylphenol has been previously described (Lee, D. H.; Kim S. Y.; Hong, J. Angew. Chem. Int. Ed. 2004, 43, 4777-4780). The representative procedure for the Becker-Adler/Diels-Alder reaction (Chapter Two, Table 1.1) was followed with 2,6-bis(hydroxymethyl)-4-phenylphenol (58 mg, 0.25 mmol) and styrene (575 μl, 5.0 mmol). The reaction was run in the mixture of THF (200 μl) and CHCl₃ (1 ml) overnight. The crude oil was purified by flash column chromatography (100:1-4:1 hexane:EtOAc) to obtain Diels-Alder product 77 as a white solid (75 mg, 90%). ₁H NMR (600 MHz, CDCl₃): δ 7.57-7.60 (m, 2H), 7.47-7.52 (m, 2H), 7.42-7.47 (m, 1H), 7.26-7.33 (m, 3H), 7.20-7.24 (m, 2H), 6.30 (d, J=1.46 Hz, 1H), 3.69 (dd, J=12.01, 7.32 Hz, 1H), 3.61 (dd, J=12.01, 7.32 Hz, 1H), 3.53 (dd, J=10.10, 5.71 Hz, 1H), 3.27-3.31 (m, 2H), 3.08 (d, J=5.86 Hz, 1H), 2.94 (ddd, J=13.55, 10.32, 3.08 Hz, 1H), 2.48 (t, J=7.32 Hz, 1H), 2.08 (ddd, J=13.47, 6.15, 2.93 Hz, 1H). ¹³C NMR (125 MHz, CDCl₃): δ 207.7, 147.0, 141.5, 136.6, 129.3, 129.0, 128.9, 128.8, 127.6, 125.2, 122.2, 62.2, 59.6, 58.3, 53.3, 43.0, 41.6, 33.8. IR (thin film): 3563 (br), 3058 (m), 2957 (m), 2876 (m), 1723 (s), 1493 (m), 1266 (m), 1052 (m), 756 (s), 731 (s), 695 (s). HRMS (ES₊): Calculated for C₂₂H₂₀O₃ [M+NH₄₊]: 350.1756, found 350.1753.

### Synthesis of Compound (78)

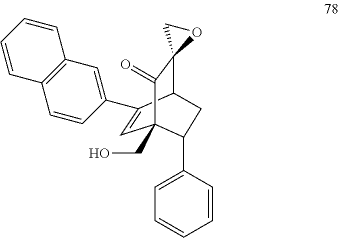

The preparation of 2,6-bis(hydroxymethyl)-4-napthalen-2-ylphenol has been previously described.₁₅ The representative procedure for the Becker-Adler/Diels-Alder reaction was followed with 2,6-bis(hydroxymethyl)-4-napthalen-2-ylphenol (70 mg, 0.25 mmol) and styrene (575 μl, 5.0 mmol). The reaction was run in THF (1 ml) overnight. The crude oil was purified by flash column chromatography (100:1-4:1 hexane:EtOAc) to obtain Diels-Alder product 78 as a white solid (80 mg, 84%). ₁H NMR (600 MHz, CDCl₃): δ 7.90-8.01 (m, 4H), 7.76 (dd, J=8.57, 1.83 Hz, 1H), 7.55-7.61 (m, 2H), 7.22-7.33 (m, 5H), 6.47 (d, J=1.90 Hz, 1H), 3.75 (dd, J=12.01, 7.03 Hz, 1H), 3.65 (dd, J=12.15, 6.88 Hz, 1H), 3.58 (dd, J=10.10, 5.71 Hz, 1H), 3.47 (dd, J=5.13, 2.64 Hz, 1H), 3.31 (d, J=6.00 Hz, 1H), 3.12 (d, J=6.00 Hz, 1H), 3.01 (ddd, J=13.58, 10.29, 2.93 Hz, 1H), 2.55 (t, J=7.18 Hz, 1H), 2.15 (ddd, J=13.69, 5.78, 2.64 Hz, 1H). ¹³C NMR (125 MHz, CDCl₃): δ 207.7, 146.8, 141.5, 133.65, 133.58, 133.56, 129.2, 128.9, 128.8, 128.5, 128.0, 127.6, 127.1, 126.9, 124.0, 123.1, 122.6, 62.2, 59.8, 58.4, 53.3, 43.3, 41.5, 33.8. IR (thin film): 3563 (br), 3057 (m), 2925 (m), 1725 (s), 1492 (m), 1458 (m), 1389 (m), 1265 (m), 1053 (m), 813 (s), 764 (s), 734 (s), 701 (s). HRMS (ES₊): Calculated for C₂₆H₂₂O₃ [M+NH₄₊]: 400.1913, found 400.1911.

### Synthesis of Compound (79)

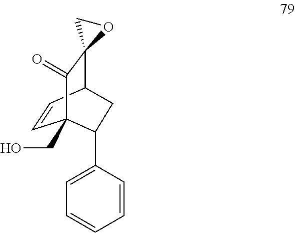

2,6-Bis(hydroxymethyl)phenol was prepared via NaBH₄ reduction of 2-hydroxyisophthalaldehyde. To a solution of 2,6-bis(hydroxymethyl)phenol (108 mg, 0.7 mmol) and styrene (1.6 ml, 14.0 mmol) in MeOH (7.0 ml) was added a solution of NaIO₄ (225 mg, 1.05 mmol) in water (1.5 ml) over 4 hours with the aid of a syringe pump at 50° C. The reaction was stirred at that temperature overnight. The volatile was removed under reduced pressure, and the remaining mixture was partitioned between water and CH₂Cl₂. The aqueous layer was extracted with CH₂Cl₂, dried over Na₂SO₄, and evaporated in vacuo. The crude product was purified by flash column chromatography (100:1-1:1 hexane:EtOAc) to obtain Diels-Alder product 79 as oil (80 mg, 45%). ₁H NMR (600 MHz, CDCl₃): δ 7.18-7.38 (m, 5H), 6.14 (s, 1H), 6.13 (s, 1H), 3.63 (dd, J=12.08, 7.25 Hz, 1H), 3.54 (dd, J=12.15, 7.18 Hz, 1H), 3.43 (dd, J=10.10, 6.00 Hz, 1H), 3.27 (d, J=6.15 Hz, 1H), 2.98 (d, J=6.15 Hz, 1H), 2.83 (ddd, J=13.33, 10.18, 2.86 Hz, 1H), 2.75-2.79 (m, 1H), 2.46 (t, J=7.25 Hz, 1H), 1.97 (ddd, J=13.44, 5.97, 2.56 Hz, 1H). ¹³C NMR (125 MHz, CDCl₃): δ 208.1, 141.7, 136.0, 129.3, 128.8, 128.7, 127.1, 84.8, 67.7, 62.0, 58.9, 58.1, 57.2, 42.0, 33.7.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A compound, or a pharmaceutically acceptable form thereof, having the formula: 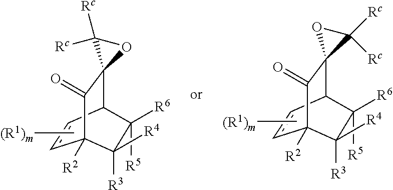
wherein
each instance of R¹ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;
R², R³, R⁴, R⁵, and R⁶, are, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;
or R¹ and R² together form a 5- to 6-membered, substituted or unsubstituted, carbocyclic or heterocyclic ring; R² and R³ together form a 5- to 6-membered, substituted or unsubstituted, carbocyclic or heterocyclic ring; R³ and R⁴ together form a 5- to 6-membered, substituted or unsubstituted, carbocyclic or heterocyclic ring; R³ and R⁵ together form a 5- to 6-membered, substituted or unsubstituted, carbocyclic or heterocyclic ring; R⁵ and R⁶ together form a 5- to 6-membered, substituted or unsubstituted, carbocyclic or heterocyclic ring; or R⁴ and R⁶ together form a 5- to 6-membered, substituted or unsubstituted, carbocyclic or heterocyclic ring;
each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl; [symbol]
corresponds to a single or double bond;
m is 0, 1, 2, 3, or 4; and
with the proviso that 5-bromo-endo-7-phenyl-exo-3-spiroepoxybicyclo[2.2.2]oct-5-en-2-one and 5-bromo-exo-7-phenyl-exo-3-spiroepoxybicyclo[2.2.2]oct-5-en-2-one are specifically excluded.

2. The compound according to claim 1, wherein said compound corresponds to the formulae:

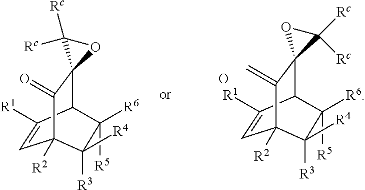

3. The compound according to claim 2, wherein said compound corresponds to the formulae:

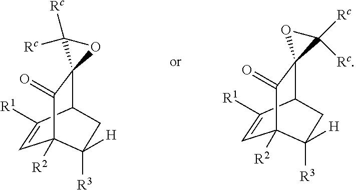

4. The compound according to claim 3, wherein said compound corresponds to the formulae: 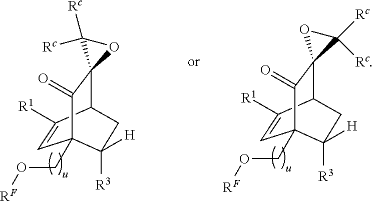
wherein R^{F} is hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or a suitable hydroxyl protecting group; and
u is 1, 2, 3, 4, 5, or 6.

5. The compound according to claim 4, wherein R^{F} is hydrogen.

6. The compound according to claim 4, wherein u is 1.

7. The compound according to claim 1, wherein R⁴ is hydrogen.

8. The compound according to claim 1, wherein R⁵ is hydrogen.

9. The compound according to claim 1, wherein R⁶ is hydrogen.

10. The compound according to claim 1, wherein R^{C} is hydrogen.

11. The compound according to claim 1, wherein R¹ is halogen.

12. The compound according to claim 1, wherein R¹ is iodo.

13. The compound according to claim 1, wherein R¹ is bromo.

14. (canceled)

15. (canceled)

16. The compound according to claim 1, wherein R² is hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl.

17. The compound according to claim 16, wherein R² is hydrogen; halogen; —WR^{D}; —CH₂WR^{D}; —CH₂CH₂WR^{D}; —CH₂CH₂CH₂WR^{D}; or —CH₂CH₂CH₂CH₂WR^{D}, wherein W is —O—, —S—, or —N(R^{W})—; R_{W} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or a suitable amino protecting group; and R^{D} is hydrogen, cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or a suitable hydroxyl, thiol, or amino protecting group, or R^{D} and R^{W} together form a 5- to 6-membered heterocyclic ring.

18. The compound according to claim 17, wherein R^{D} and R^{W} are hydrogen.

19. The compound according to claim 17, wherein W is —O—.

20. The compound according to claim 1, wherein R³ is substituted or unsubstituted aryl; substituted or unsubstituted heteroaryl; cyclic or acyclic, substituted or unsubstituted alkyloxy; or cyclic or acyclic, substituted or unsubstituted heteroalkyloxy,

21. The compound according to claim 20, wherein R³ corresponds to the formula: 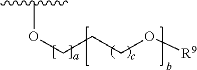
wherein
R⁹ is hydrogen; a substituted or unsubstituted aliphatic; a substituted or unsubstituted aryl; or a substituted or unsubstituted heteroaryl; or a suitable hydroxyl protecting group;
a is 0, 1, 2, 3, 4, 5, or 6;
b is 0, 1, 2, or 3; and
c is 1, 2, 3, 4, 5, or 6.

22. The compound according to claim 20, wherein R³ corresponds to the formula: 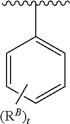
wherein
R⁸ is halogen; substituted or unsubstituted hydroxyl; substituted or unsubstituted thiol; substituted or unsubstituted amino; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; nitro; cyano; isocyano; azido; —SO₃R^{E}; —SO₂R^{E}, —SOR^{E}, —C(═O)R^{E}, —C(═O)OR^{E}, —C(═O)N(R^{E})₂, —C(═NR^{E})N(R^{E})₂, wherein each instance of R^{E} is, independently, hydrogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or two R^{E} groups taken together form a 5- to 6-membered heterocyclic ring; and
t is 0, 1, 2, 3, 4, or 5.

23. The compound according to claim 22, wherein R⁸ is halogen; substituted or unsubstituted hydroxyl; or cyclic or acyclic, substituted or unsubstituted, branched or unbranched aliphatic.

24. The compound according to claim 22, wherein R³ corresponds to the formulae:

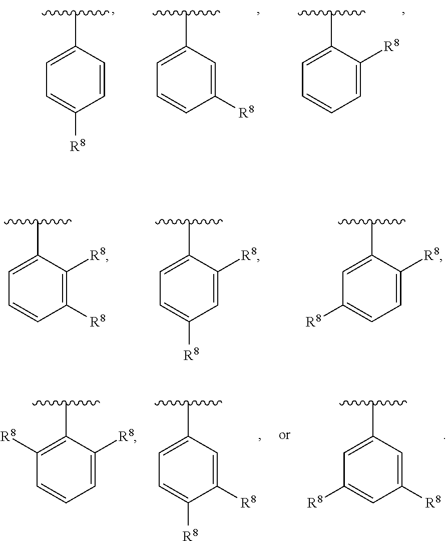

25. The compound according to claim 1, wherein said compound corresponds to the formulae: 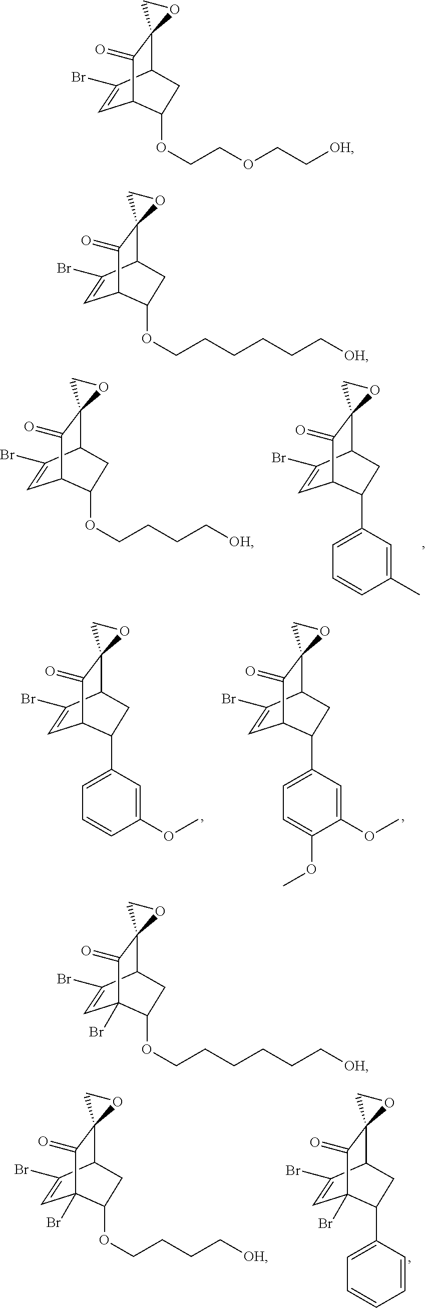 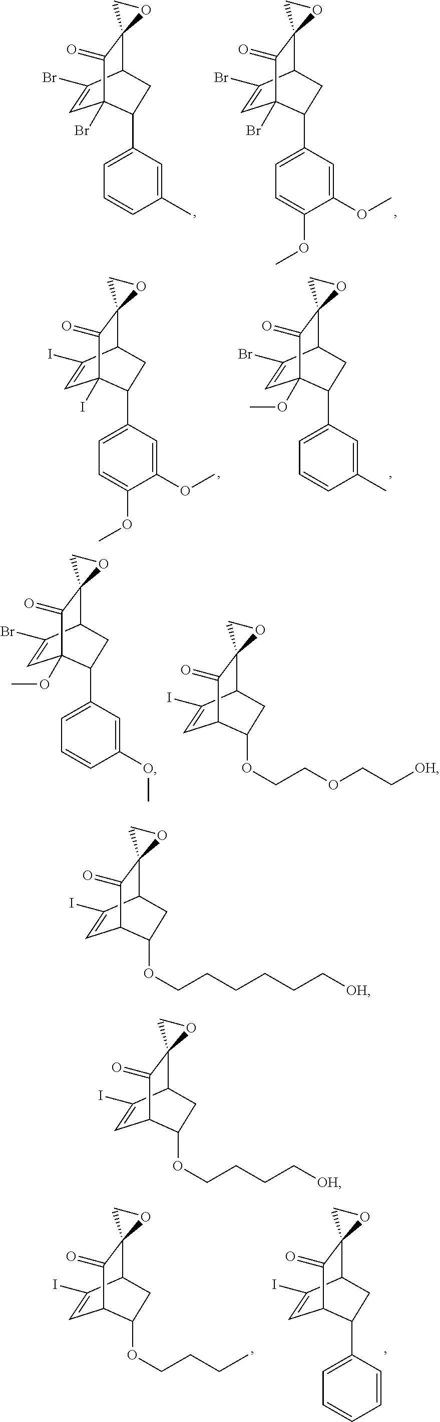 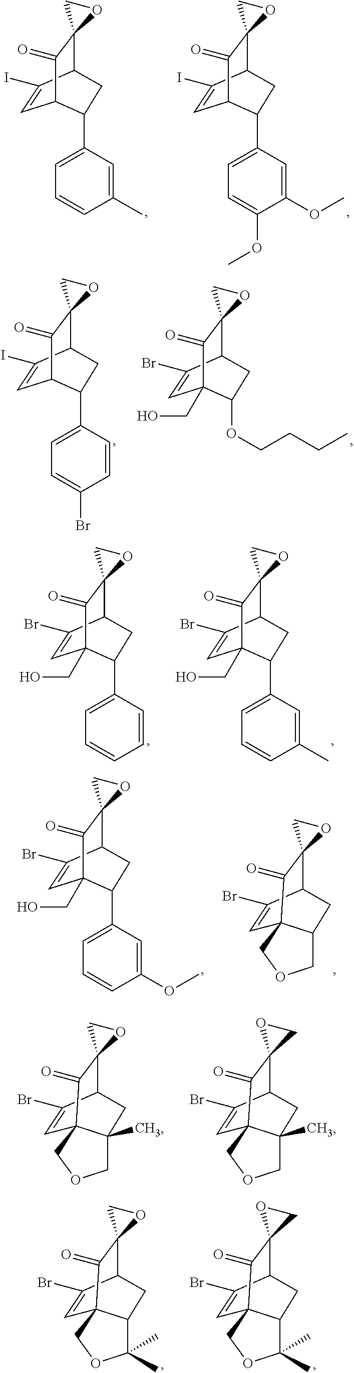 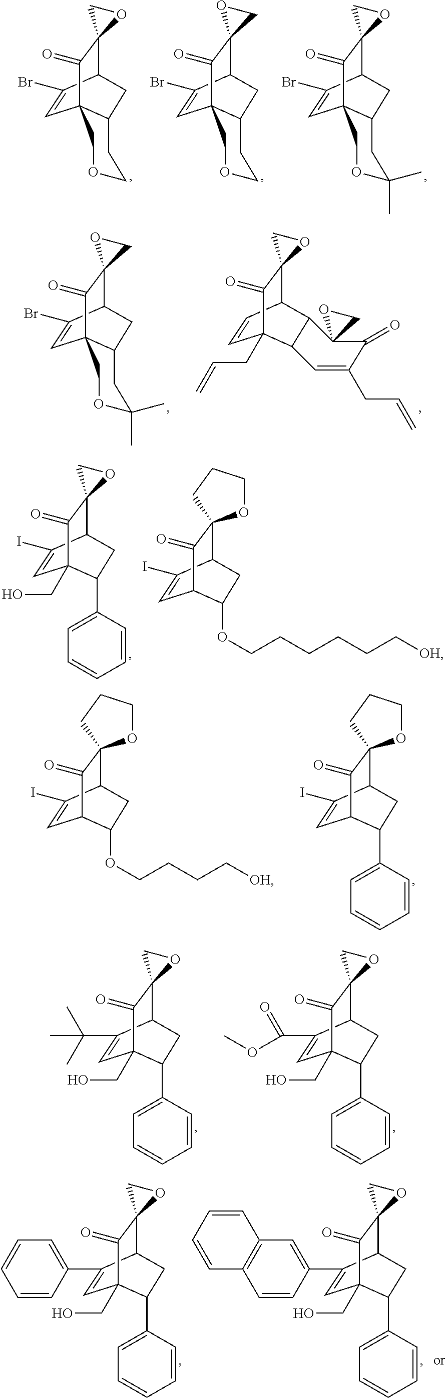 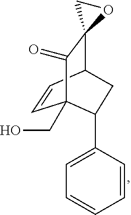
or a pharmaceutically acceptable form thereof.

26. (canceled)

27. (canceled)

28. (canceled)

29. A pharmaceutical composition comprising a therapeutically effective amount of a compound of claim 1 and a pharmaceutically acceptable excipient.

30. A method of treating a subject diagnosed with or being susceptible to a viral infection comprising administering to a therapeutically effective amount of a compound, or a pharmaceutically acceptable form thereof, having the formula: 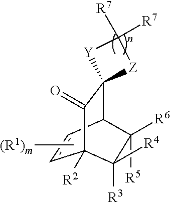
wherein
each instance of R¹ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;
R², R³, R⁴, R⁵, and R⁶, are, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or substituted or unsubstituted acyl;
or R¹ and R² together form a 5- to 6-membered, substituted or unsubstituted, carbocyclic or heterocyclic ring; R² and R³ together form a 5- to 6-membered, substituted or unsubstituted, carbocyclic or heterocyclic ring; R³ and R⁴ together form a 5- to 6-membered, substituted or unsubstituted, carbocyclic or heterocyclic ring; R³ and R⁵ together form a 5- to 6-membered, substituted or unsubstituted, carbocyclic or heterocyclic ring; R⁵ and R⁶ together form a 5- to 6-membered, substituted or unsubstituted, carbocyclic or heterocyclic ring; or R⁴ and R⁶ together form a 5- to 6-membered, substituted or unsubstituted, carbocyclic or heterocyclic ring;
each instance of R⁷ is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; or substituted or unsubstituted heteroaryl; substituted or unsubstituted acyl; or two R⁷ groups taken together form an oxo (═O), an imino (═NH), or a thiooxo (═S) group;
Y and Z are, independently, —O—, —S—, —N(R^{C})—, or —C(R^{C})₂—, wherein each instance of R^{C} is, independently, hydrogen; halogen; substituted or unsubstituted amino; substituted or unsubstituted thiol; substituted or unsubstituted hydroxyl; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; or two R^{C} groups taken together form an oxo (═O), an imino (═NH), or a thiooxo (═S) group; [symbol]
corresponds to a single or double bond;
m is 0, 1, 2, 3, or 4; and
n is 0, 1, 2, or 3.

31.-60. (canceled)

61. A method of treating a subject diagnosed with or being susceptible to a viral infection comprising administering to a therapeutically effective amount of a compound, or a pharmaceutically acceptable form thereof, having the formula: 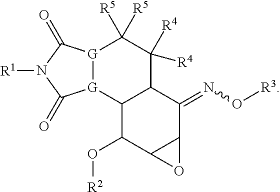
wherein
each instance of G is, independently, —N— or —(CH)—;
R¹ is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or a suitable amino protecting group;
R² is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or a suitable hydroxyl protecting group;
R³ is hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; cyclic or acyclic, substituted or unsubstituted heteroaryl; or a suitable hydroxyl protecting group; and
each instance of R⁴ and R⁵ is, independently, hydrogen; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted aryl; or cyclic or acyclic, substituted or unsubstituted heteroaryl.

61-81. (canceled)
